(19) United States
(12) Patent Application Publication
(10) Pub. No.: US 2002/0059663 A1
(43) Pub. Date: May 16, 2002

(54) Expressed sequences of arabidopsis thaliana

(76) Inventors: Jorn Gorlach, Durham, NC (US); Yong-Qiang An, San Diego, CA (US); Carol M. Hamilton, Apex, NC (US); Jennifer L. Price, Raleigh, NC (US); Tracy M. Raines, Durham, NC (US); Yang Yu, Matinsville, NJ (US); Joshua G. Rameaka, Durham, NC (US); Amy Page, Durham, NC (US); Abraham V. Mathew, Cary, NC (US); Brooke L. Ledford, Holly Springs, NC (US); Jeffrey P. Woessner, Hillsborough, NC (US); William David Haas, Durham, NC (US); Carlos A. Garcia, Carrboro, NC (US); Maja Kricker, Pittsboro, NC (US); Ted Slater, Apex, NC (US); Keith R. Davis, Durham, NC (US); Keith Allen, Cary, NC (US); Neil Hoffman, Chapel Hill, NC (US); Patrick Hurban, Raleigh, NC (US)

Correspondence Address: PARADIGM GENETICS, INC, 104 ALEXANDER DRIVE, BUILDING 2, P O BOX 14528, RTP, NC, 277094528

(21) Appl. No.: 09/770,149

(22) Filed: Jan. 26, 2001

Related U.S. Application Data
(60) Provisional application No. 60/178,506, filed on Jan. 27, 2000.

(51) Int. Cl. A01H 5/00; C12N 15/82

(52) U.S. Cl. 800/298; 536/23.6; 435/320.1; 530/350; 435/419

(57) ABSTRACT

Isolated nucleotide compositions and sequences are provided for Arabidopsis thaliana genes. The nucleic acid compositions find use in identifying homologous or related genes; in producing compositions that modulate the expression or function of its encoded protein, mapping functional regions of the protein; and in studying associated physiological pathways. The genetic sequences may also be used for the genetic manipulation of cells, particularly of plant cells. The encoded gene products and modified organisms are useful for screening of biologically active agents, e.g. fungicides, insecticides, etc.; for elucidating biochemical pathways; and the like.

### CROSS -REFERENCE TO RELATED APPLICATION

This application claims the benefit of U.S. Provisional Application No. 60/178,506 Filed Jan. 27, 2000.

### FIELD OF INVENTION

The invention is in the field of polynucleotide sequences of a plant, particularly sequences expressed in arabidopsis thaliana.

### BACKGROUND OF THE INVENTION

Plants and plant products have vast commercial importance in a wide variety of areas including food crops for human and animal consumption, flavor enhancers for food, and production of specialty chemicals for use in products such as medicaments and fragrances. In considering food crops for humans and livestock, genes such as those involved in a plants resistance to insects, plant viruses, and fungi; genes involved in pollination; and genes whose products enhance the nutritional value of the food, are of major importance. A number of such genes have been described, see, for example, McCaskill and Croteau (1999) Nature Biotechnol. 17:31-36.

Despite recent advances in methods for identification, cloning, and characterization of genes, much remains to be learned about plant physiology in general, including how plants produce many of the above-mentioned products; mechanisms for resistance to herbicides, insects, plant viruses, fungi; elucidation of genes involved in specific biosynthetic pathways; and genes involved in environmental tolerance, e.g., salt tolerance, drought tolerance, or tolerance to anaerobic conditions.

Arabidopsis thaliana is a model system for genetic, molecular and biochemical studies of higher plants. Features of this plant that make it a model system for genetic and molecular biology research include a small genome size, organized into five chromosomes and containing an estimated 20,000 genes, a rapid life cycle, prolific seed production and, since it is small, it can easily be cultivation in limited space. A. thaliana is a member of the mustard family (Brassicaceae) with a broad natural distribution throughout Europe, Asia, and North America. Many different ecotypes have been collected from natural populations and are available for experimental analysis. The entire life cycle, including seed germination, formation of a rosette plant, bolting of the main stem, flowering, and maturation of the first seeds, is completed in 6 weeks. A large number of mutant lines are available that affect nearly all aspects of its growth. These features greatly facilitate the isolation of fundamentally interesting and potentially important genes for agronomic development

Most gene products from higher plants exhibit adequate sequence similarity to deduced amino acid sequences of other plant genes to permit assignment of probable gene function, if it is known, in any higher plant. It is likely that there will be very few protein-encoding angiosperm genes that do not have orthologs or paralogs in Arabidopsis. The developmental diversity of higher plants may be largely due to changes in the cis-regulatory sequences of transcriptional regulators and not in coding sequences.

Many advances reported over the past few years offer clear evidence that this plant is not only a very important model species for basic research, but also extremely valuable for applied plant scientists and plant breeders. Knowledge gained from Arabidopsis can be used directly to develop desired traits in plants of other species.

### Relevant Literature

Cold Spring Harbor Monograph 27 (1994) E. M. Meyerowitz and C. R. Somerville, eds. (CSH Laboratory Press). Annual Plant Reviews, Vol. 1: Arabidopsis (1998) M. Anderson and J. A. Roberts, eds. (CRC Press). Methods in Molecular Biology: Arabidopsis Protocols, Vol. 82 (1997) J. M. Martinez-Zapater and J. Salinas, eds. (CRC Press).

Mayer et al (1999) Nature 402(6763):769-77; “Sequence and analysis of chromosome 4 of the plant Arabidopsis thaliana.” Lin et al. (1999) 402 (6763):761-8, “Sequence and analysis of chromosome 2 of the plant Arabidopsis thaliana.” Meinke et al. (1998) Science 282:662-682, “Arabidopsis thaliana: a model plant for genome analysis”. Somerville and Somerville (1999) Science 285:380-383, “Plant functional genomics”. Mozo et al. (1999) Nat. Genet. 22:271-275, “A complete BAC-based physical map of the Arabidopsis thaliana genome”.

### SUMMARY OF THE INVENTION

Novel nucleic acid sequences of Arabidopsis thaliana, their encoded polypeptides and variants thereof, genes corresponding to these nucleic acids, and proteins expressed by the genes, are provided.

The invention also provides diagnostic, prophylactic and therapeutic agents employing such novel nucleic acids, their corresponding genes or gene products, including expression constructs, probes, antisense constructs, and the like. The genetic sequences may also be used for the genetic manipulation of plant cells, particularly dicotyledonous plants. The encoded gene products and modified organisms are useful for introducing or improving disease resistance and stress tolerance into plants; screening of biologically active agents, e.g. fungicides, etc.; for elucidating biochemical pathways; and the like.

In one embodiment of the invention, a nucleic acid is provided that comprises a start codon; an optional intervening sequence; a coding sequence capable of hybridizing under stringent conditions as set forth in SEQ ID NO:1 to 999; and an optional terminal sequence, wherein at least one of said optional sequences is present. Such a nucleic acid may correspond to naturally occurring Arabidopsis expressed sequences.

### DETAILED DESCRIPTION OF THE INVENTION

Novel nucleic acid sequences from Arabidopsis thaliana, their encoded polypeptides and variants thereof, genes corresponding to these nucleic acids and proteins expressed by the genes are provided. The invention also provides agents employing such novel nucleic acids, their corresponding genes or gene products, including expression constructs, probes, antisense constructs, and the like. The nucleotide sequences are provided in the attached SEQLIST.

Sequences include, but are not limited to, sequences that encode resistance proteins; sequences that encode tolerance factors; sequences encoding proteins or other factors that are involved, directly or indirectly in biochemical pathways such as metabolic or biosynthetic pathways, sequences involved in signal transduction, sequences involved in the regulation of gene expression, structural genes, and the like. Biosynthetic pathways of interest include, but are not limited to, biosynthetic pathways whose product (which may be an end product or an intermediate) is of commercial, nutritional, or medicinal value.

The sequences may be used in screening assays of various plant strains to determine the strains that are best capable of withstanding a particular disease or environmental stress. Sequences encoding activators and resistance proteins may be introduced into plants that are deficient in these sequences. Alternatively, the sequences may be introduced under the control of promoters that are convenient for induction of expression. The protein products may be used in screening programs for insecticides, fungicides and antibiotics to determine agents that mimic or enhance the resistance proteins. Such agents may be used in improved methods of treating crops to prevent or treat disease. The protein products may also be used in screening programs to identify agents which mimic or enhance the action of tolerance factors. Such agents may be used in improved methods of treating crops to enhance their tolerance to environmental stresses.

Still other embodiments of the invention provide methods for enhancing or inhibiting production of a biosynthetic product in a plant by introducing a nucleic acid of the invention into a plant cell, where the nucleic acid comprises sequences encoding a factor which is involved, directly or indirectly in a biosynthetic pathway whose products are of commercial, nutritional, or medicinal value include any factor, usually a protein or peptide, which regulates such a biosynthetic pathway; which is an intermediate in such a biosynthetic pathway; or which in itself is a product that increases the nutritional value of a food product; or which is a medicinal product; or which is any product of commercial value.

Transgenic plants containing the antisense nucleic acids of the invention are useful for identifying other mediators that may induce expression of proteins of interest; for establishing the extent to which any specific insect and/or pathogen is responsible for damage of a particular plant; for identifying other mediators that may enhance or induce tolerance to environmental stress; for identifying factors involved in biosynthetic pathways of nutritional, commercial, or medicinal value; or for identifying products of nutritional, commercial, or medicinal value.

In still other embodiments, the invention provides transgenic plants constructed by introducing a subject nucleic acid of the invention into a plant cell, and growing the cell into a callus and then into a plant; or, alternatively by breeding a transgenic plant from the subject process with a second plant to form an F1 or higher hybrid. The subject transgenic plants and progeny are used as crops for their enhanced disease resistance, enhanced traits of interest, for example size or flavor of fruit, length of growth cycle, etc., or for screening programs, e.g. to determine more effective insecticides, etc; used as crops which exhibit enhanced tolerance environmental stress; or used to produce a factor.

Those skilled in the art will recognize the agricultural advantages inherent in plants constructed to have either increased or decreased expression of resistance proteins; or increased or decreased tolerance to environmental factors; or which produce or over-produce one or more factors involved in a biosynthetic pathway whose product is of commercial, nutritional, or medicinal value. For example, such plants may have increased resistance to attack by predators, insects, pathogens, microorganisms, herbivores, mechanical damage and the like; may be more tolerant to environmental stress, e.g. may be better able to withstand drought conditions, freezing, and the like; or may produce a product not normally made in the plant, or may produce a product in higher than normal amounts, where the product has commercial, nutritional, or medicinal value. Plants which may be useful include dicotyledons and monocotyledons. Representative examples of plants in which the provided sequences may be useful include tomato, potato, tobacco, cotton, soybean, alfalfa, rape, and the like. Monocotyledons, more particularly grasses (Poaceae family) of interest, include, without limitation, Avena sativa (oat); Avena strigosa (black oat); Elymus (wild rye); Hordeum sp. including Hordeum vulgare (barley); Oryza sp., including Oryza glaberrima (African rice); Oryza longistaminata (long-staminate rice); Pennisetum americanum (pearl millet); Sorghum sp. (sorghum); Triticum sp., including Triticum aestivum (common wheat); Triticum durum (durum wheat); Zea mays (corn); etc.

### NUCLEIC ACID COMPOSITIONS

The following detailed description describes the nucleic acid compositions encompassed by the invention, methods for obtaining cDNA or genomic DNA encoding a full-length gene product, expression of these nucleic acids and genes; identification of structural motifs of the nucleic acids and genes; identification of the function of a gene product encoded by a gene corresponding to a nucleic acid of the invention; use of the provided nucleic acids as probes, in mapping, and in diagnosis; use of the corresponding polypeptides and other gene products to raise antibodies; use of the nucleic acids in genetic modification of plant and other species; and use of the nucleic acids, their encoded gene products, and modified organisms, for screening and diagnostic purposes.

The scope of the invention with respect to nucleic acid compositions includes, but is not necessarily limited to, nucleic acids having a sequence set forth in any one of SEQ ID NOS:1-999; nucleic acids that hybridize the provided sequences under stringent conditions; genes corresponding to the provided nucleic acids; variants of the provided nucleic acids and their corresponding genes, particularly those variants that retain a biological activity of the encoded gene product.

In one embodiment, the sequences of the invention provide a polypeptide coding sequence. The polypeptide coding sequence may correspond to a naturally expressed mRNA in Arabidopsis or other species, or may encode a fusion protein between one of the provided sequences and an exogenous protein coding sequence. The coding sequence is characterized by an ATG start codon, a lack of stop codons in-frame with the ATG, and a termination codon, that is, a continuous open frame is provided between the start and the stop codon. The sequence contained between the start and the stop codon will comprise a sequence capable of hybridizing under stringent conditions to a sequence set for in SEQ ID NO:1-999, and may comprise the sequence set forth in the Seqlist.

Other nucleic acid compositions contemplated by and within the scope of the present invention will be readily apparent to one of ordinary skill in the art when provided with the disclosure here.

The invention features nucleic acids that are derived from Arabidopsis thaliana. Novel nucleic acid compositions of the invention of particular interest comprise a sequence set forth in any one of SEQ ID NOS:1-999 or an identifying sequence thereof. An “identifying sequence” is a contiguous sequence of residues at least about 10 nt to about 20 nt in length, usually at least about 50 nt to about 100 nt in length, that uniquely identifies a nucleic acid sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from any one of SEQ ID NOS:1-999.

The nucleic acids of the invention also include nucleic acids having sequence similarity or sequence identity. Nucleic acids having sequence similarity are detected by hybridization under low stringency conditions, for example, at 50° C. and 10XSSC (0.9M NaCl/0.09M sodium citrate) and remain bound when subjected to washing at 55° C. in 1XSSC. Sequence identity can be determined by hybridization under stringent conditions, for example, at 50° C. or higher and 0.1XSSC (9 mM NaCl/0.9 mM sodium citrate). Hybridization methods and conditions are well known in the art, see U.S. Pat. No. 5,707,829. Nucleic acids that are substantially identical to the provided nucleic acid sequences, e.g. allelic variants, genetically altered versions of the gene, etc., bind to the provided nucleic acid sequences (SEQ ID NOS:1-999) under stringent hybridization conditions. By using probes, particularly labeled probes of DNA sequences, one can isolate homologous or related genes. The source of homologous genes can be any species, particularly grasses as previously described.

Preferably, hybridization is performed using at least 15 contiguous nucleotides of at least one of SEQ ID NOS:1-999. The probe will preferentially hybridize with a nucleic acid or mRNA comprising the complementary sequence, allowing the identification and retrieval of the nucleic acids of the biological material that uniquely hybridize to the selected probe. Probes of more than 15 nucleotides can be used, e.g. probes of from about 18 nucleotides up to the entire length of the provided nucleic acid sequences, but 15 nucleotides generally represents sufficient sequence for unique identification.

The nucleic acids of the invention also include naturally occurring variants of the nucleotide sequences, e.g. degenerate variants, allelic variants, etc. Variants of the nucleic acids of the invention are identified by hybridization of putative variants with nucleotide sequences disclosed herein, preferably by hybridization under stringent conditions For example, by using appropriate wash conditions, variants of the nucleic acids of the invention can be identified where the allelic variant exhibits at most about 25-30% base pair mismatches relative to the selected nucleic acid probe. In general, allelic variants contain 5-25% base pair mismatches, and can contain as little as even 2-5%, or 1-2% base pair mismatches, as well as a single base-pair mismatch.

The invention also encompasses homologs corresponding to the nucleic acids of SEQ ID NOS:1-999, where the source of homologous genes can be any related species, usually within the same genus or group. Homologs have substantial sequence similarity, e.g. at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as BLAST, described in Altschul et al., J. Mol. Biol. (1990) 215:403-10.

In general, variants of the invention have a sequence identity greater than at least about 65%, preferably at least about 75%, more preferably at least about 85%, and can be greater than at least about 90% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Wateman homology search algorithm as implemented in MPSRCH program (Oxford Molecular) using an affine gap search with the following search parameters: gap open penalty, 12; and gap extention penalty, 1.

The subject nucleic acids can be cDNAs or genomic DNAs, as well as fragments thereof, particularly fragments that encode a biologically active gene product and/or are useful in the methods disclosed herein. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kb or smaller; and substantially free of flanking chromosomal sequence. The genomic DNA flanking the coding region, either 3′ and 5′, or internal regulatory sequences as sometimes found in introns, contains sequences required for expression.

The nucleic acid compositions of the subject invention can encode all or a part of the subject expressed polypeptides. Double or single stranded fragments can be obtained from the DNA sequence by chemically synthesizing oligonucleotides in accordance with conventional methods, by restriction enzyme digestion, by PCR amplification, etc. Isolated nucleic acids and nucleic acid fragments of the invention comprise at least about 15 up to about 100 contiguous nucleotides, or up to the complete sequence provided in SEQ ID NOS:1-999. For the most part, fragments will be of at least 15 nt, usually at least 18 nt or 25 nt, and up to at least about 50 contiguous nt in length or more.

Probes specific to the nucleic acids of the invention can be generated using the nucleic acid sequences disclosed in SEQ ID NOS:1-999 and the fragments as described above. The probes can be synthesized chemically or can be generated from longer nucleic acids using restriction enzymes. The probes can be labeled, for example, with a radioactive, biotinylated, or fluorescent tag. Preferably, probes are designed based upon an identifying sequence of a nucleic acid of one of SEQ ID NOS:1-999. More preferably, probes are designed based on a contiguous sequence of one of the subject nucleic acids that remain unmasked following application of a masking program for masking low complexity (e.g., XBLAST) to the sequence., i.e. one would select an unmasked region, as indicated by the nucleic acids outside the poly-n stretches of the masked sequence produced by the masking program.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The nucleic acids of the invention can be provided as a linear molecule or within a circular molecule. They can be provided within autonomously replicating molecules (vectors) or within molecules without replication sequences. They can be regulated by their own or by other regulatory sequences, as is known in the art. The nucleic acids of the invention can be introduced into suitable host cells using a variety of techniques which are available in the art, such as transferring polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used to, for example, produce polypeptides, as probes for the detection of mRNA of the invention in biological samples, e.g. extracts of cells, to generate additional copies of the nucleic acids, to generate ribozymes or antisense oligonucleotides, and as single stranded DNA probes or as triple-strand forming oligonucleotides. The probes described herein can be used to, for example, determine the presence or absence of the nucleic acid sequences as shown in SEQ ID NOS:1-999 or variants thereof in a sample. These and other uses are described in more detail below.

### USE OF NUCLEIC ACIDS AS CODING SEQUENCES

Naturally occurring Arabidopsis polypeptides or fragments thereof are encoded by the provided nucleic acids. Methods are known in the art to determine whether the complete native protein is encoded by a candidate nucleic acid sequence. Where the provided sequence encodes a fragment of a polypeptide, methods known in the art may be used to determine the remaining sequence. These approaches may utilize a bioinformatics approach, a cloning approach, extension of mRNA species, etc.

Substantial genomic sequence is available for Arabidopsis, and may be exploited for determining the complete coding sequence corresponding to the provided sequences. The region of the chromosome to which a given sequence is located may be determined by hybridization or by database searching. The genomic sequence is then searched upstream and downstream for the presence of intron/exon boundaries, and for motifs characteristic of transcriptional start and stop sequences, for example by using Genscan (Burge and Karlin (1997) J. Mol. Biol. 268:78-94); or GRAIL (Uberbacher and Mural (1991) P.N.A.S. 88:11261-1265).

Alternatively, nucleic acid having a sequence of one of SEQ ID NOS:1-999, or an identifying fragment thereof, is used as a hybridization probe to complementary molecules in a cDNA library using probe design methods, cloning methods, and clone selection techniques as known in the art. Libraries of cDNA are made from selected cells. The cells may be those of A. thaliana, or of related species. In some cases it will be desirable to select cells from a particular stage, e.g. seeds, leaves, infected cells, etc.

Techniques for producing and probing nucleic acid sequence libraries are described, for example, in Sambrook et al., Molecular Cloning: A Laboratory Manual, 2^{nd} Ed., (1989) Cold Spring Harbor Press, Cold Spring Harbor, N.Y.; and Current Protocols in Molecular Biology, (1987 and updates) Ausubel et al., eds. The cDNA can be prepared by using primers based on sequence from SEQ ID NOS:1-999. In one embodiment, the cDNA library can be made from only poly-adenylated mRNA. Thus, poly-T primers can be used to prepare cDNA from the mRNA.

Members of the library that are larger than the provided nucleic acids, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2^{nd} Ed., (1989) Cold Spring Harbor Press, Cold Spring Harbor, N.Y. In order to obtain additional sequences 5′ to the end of a partial cDNA, 5′ RACE (PCR Protocols: A Guide to Methods and Applications, (1990) Academic Press, Inc.) may be performed.

Genomic DNA is isolated using the provided nucleic acids in a manner similar to the isolation of full-length cDNAs. Briefly, the provided nucleic acids, or portions thereof, are used as probes to libraries of genomic DNA. Preferably, the library is obtained from the cell type that was used to generate the nucleic acids of the invention, but this is not essential. Such libraries can be in vectors suitable for carrying large segments of a genome, such as P1 or YAC, as described in detail in Sambrook et al., 9.4-9.30. In order to obtain additional 5′ or 3′ sequences, chromosome walking is performed, as described in Sambrook et al., such that adjacent and overlapping fragments of genomic DNA are isolated. These are mapped and pieced together, as is known in the art, using restriction digestion enzymes and DNA ligase.

PCR methods may be used to amplify the members of a cDNA library that comprise the desired insert. In this case, the desired insert will contain sequence from the full length cDNA that corresponds to the instant nucleic acids. Such PCR methods include gene trapping and RACE methods. Gene trapping entails inserting a member of a cDNA library into a vector. The vector then is denatured to produce single stranded molecules. Next, a substrate-bound probe, such a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the nucleic acid sequences of the invention. Random primers or primers specific to the library vector can be used to amplify the trapped cDNA. Such gene trapping techniques are described in Gruber et al., WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends”, or RACE, is a PCR method of amplifying cDNAs from a number of different RNAs. The cDNAs are ligated to an oligonucleotide linker, and amplified by PCR using two primers. One primer is based on sequence from the instant nucleic acids, for which full length sequence is desired, and a second primer comprises sequence that hybridizes to the oligonucleotide linker to amplify the cDNA. A description of this methods is reported in WO 97/19110. A common primer may be designed to anneal to an arbitrary adaptor sequence ligated to cDNA ends. When a single gene-specific RACE primer is paired with the common primer, preferential amplification of sequences between the single gene specific primer and the common primer occurs. Commercial cDNA pools modified for use in RACE are available.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function. As an alternative method to obtaining DNA or RNA from a biological material, nucleic acid comprising nucleotides having the sequence of one or more nucleic acids of the invention can be synthesized.

### EXPRESSION OF POLYPEPTIDES

The provided nucleic acid, e.g. a nucleic acid having a sequence of one of SEQ ID NOS:1-999), the corresponding cDNA, the polypeptide coding sequence as described above, or the full-length gene is used to express a partial or complete gene product. Constructs of nucleic acids having sequences of SEQ ID NOS:1-999 can be generated by recombinant methods, synthetically, or in a single-step assembly of a gene and entire plasmid from large numbers of oligodeoxyribonucleotides is described by, e.g. Stemmer et al., Gene (Amsterdam) (1995) 164(1):49-53.

Appropriate nucleic acid constructs are purified using standard recombinant DNA techniques as described in, for example, Sambrook et al., Molecular Cloning: A Laboratory Manual, 2^{nd} Ed., (1989) Cold Spring Harbor Press, Cold Spring Harbor, N.Y. The gene product encoded by a nucleic acid of the invention is expressed in any expression system, including, for example, bacterial, yeast, insect, amphibian and mammalian systems.

The subject nucleic acid molecules are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole organism or person. The choice of appropriate vector is well within the skill of the art. Many such vectors are available commercially.

The nucleic acids set forth in SEQ ID NOS:1-999 or their corresponding full-length nucleic acids are linked to regulatory sequences as appropriate to obtain the desired expression properties. These can include promoters attached either at the 5′ end of the sense strand or at the 3′ end of the antisense strand, enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the nucleic acids or nucleic acids of the invention, the resulting replicated nucleic acid, RNA, expressed protein or polypeptide, is within the scope of the invention as a product of the host cell or organism. The product is recovered by any appropriate means known in the art.

### IDENTIFICATION OF FUNCTIONAL AND STRUCTURAL MOTIFS

Translations of the nucleotide sequence of the provided nucleic acids, cDNAs or full genes can be aligned with individual known sequences. Similarity with individual sequences can be used to determine the activity of the polypeptides encoded by the nucleic acids of the invention. Also, sequences exhibiting similarity with more than one individual sequence can exhibit activities that are characteristic of either or both individual sequences.

The six possible reading frames may be translated using programs such as GCG pepdata, or GCG Frames (Wisconsin Package Version 10.0, Genetics Computer Group (GCG), Madison, Wis., USA. ). Programs such as ORFFinder (National Center for Biotechnology Information (NCBI) a division of the National Library of Medicine (NLM) at the National Institutes of Health (NIH) http://www.ncbi.nlm.nih.gov/) may be used to identify open reading frames (ORFs) in sequences. ORF finder identifies all possible ORFs in a DNA sequence by locating the standard and alternative stop and start codons. Other ORF identification programs include Genie (Kulp et al. (1996).

A generalized Hidden Markov Model may be used for the recognition of genes in DNA. (ISMB-96, St. Louis, Mo., AAAI/MIT Press; Reese et al. (1997), “Improved splice site detection in Genie”. Proceedings of the First Annual International Conference on Computational Molecular Biology RECOMB 1997, Santa Fe, N.M., ACM Press, New York., P. 34.); BESTORF—Prediction of potential coding fragment in human or plant EST/mRNA sequence data using Markov Chain Models; and FGENEP—Multiple genes structure prediction in plant genomic DNA (Solovyev et al. (1995) Identification of human gene structure using linear discriminant functions and dynamic programming. In Proceedings of the Third International Conference on Intelligent Systems for Molecular Biology eds. Rawling et al. Cambridge, England, AAAI Press,367-375.; Solovyev et al. (1994) Nucl. Acids Res. 22(24):5156-5163; Solovyev et al,. The prediction of human exons by oligonucleotide composition and discriminant analysis of spliceable open reading frames, in: The Second International conference on Intelligent systems for Molecular Biology (eds. Altman et al.), AAAI Press, Menlo Park, Calif. (1994, 354-362) Solovyev and Lawrence, Prediction of human gene structure using dynamic programming and oligonucleotide composition, In: Abstracts of the 4th annual Keck symposium. Pittsburgh, 47,1993; Burge and Karlin (1997) J. Mol. Biol. 268:78-94; Kulp et al. (1996) Proc. Conf. on Intelligent Systems in Molecular Biology '96, 134-142).

The full length sequences and fragments of the nucleic acid sequences of the nearest neighbors can be used as probes and primers to identify and isolate the full length sequence corresponding to provided nucleic acids. Typically, a selected nucleic acid is translated in all six frames to determine the best alignment with the individual sequences. These amino acid sequences are referred to, generally, as query sequences, which are aligned with the individual sequences. Suitable databases include Genbank, EMBL, and DNA Database of Japan (DDBJ).

Query and individual sequences can be aligned using the methods and computer programs described above, and include BLAST, available by ftp at ftp://ncbi.nlm.nih.gov/.

Gapped BLAST and PSI-BLAST are useful search tools provided by NCBI. (version 2.0) (Altschul et al., 1997). Position-Specific Iterated BLAST (PSI-BLAST) provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. PSI-BLAST may be iterated until no new significant alignments are found. The Gapped BLAST algorithm allows gaps (deletions and insertions) to be introduced into the alignments that are returned. Allowing gaps means that similar regions are not broken into several segments. The scoring of these gapped alignments tends to reflect biological relationships more closely. The Smith-Waterman is another algorithm that produces local or global gapped sequence alignments, see Meth. Mol. Biol. (1997) 70: 173-187. Also, the GAP program using the Needleman and Wunsch global alignment method can be utilized for sequence alignments.

Results of individual and query sequence alignments can be divided into three categories, high similarity, weak similarity, and no similarity. Individual alignment results ranging from high similarity to weak similarity provide a basis for determining polypeptide activity and/or structure. Parameters for categorizing individual results include: percentage of the alignment region length where the strongest alignment is found, percent sequence identity, and e value.

The percentage of the alignment region length is calculated by counting the number of residues of the individual sequence found in the region of strongest alignment, e.g. contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is: 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%

E value is the probability that the alignment was produced by chance. For a single alignment, the e value can be calculated according to Karlin et al., Proc. Natl. Acad. Sci. (1990) 87:2264 and Karlin et al., Proc. Natl. Acad. Sci. (1993) 90. The e value of multiple alignments using the same query sequence can be calculated using an heuristic approach described in Altschul et al., Nat. Genet. (1994) 6:119. Alignment programs such as BLAST program can calculate the e value.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences. The boundaries of the region where the sequences align can be determined according to Doolittle, supra; BLAST or FASTA programs; or by determining the area where sequence identity is highest.

In general, in alignment results considered to be of high similarity, the percent of the alignment region length is typically at least about 55% of total length query sequence; more typically, at least about 58%; even more typically; at least about 60% of the total residue length of the query sequence. Usually, percent length of the alignment region can be as much as about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. The query sequence is considered to have a high similarity with a profile sequence when the p value is less than or equal to 10⁻². Confidence in the degree of similarity between the query sequence and the profile sequence increases as the p value become smaller.

In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically; at least about 25 amino acid residues in length. Usually, length of the alignment region can be as much as about 30 amino acid residues; more usually, as much as about 40; even more usually, as much as about 60 amino acid residues. Further, for weak similarity, the region of alignment, typically, exhibits at least about 35% of sequence identity; more typically, at least about 40%; even more typically; at least about 45% sequence identity. Usually, percent sequence identity can be as much as about 50%; more usually, as much as about 55%; even more usually, as much as about 60%.

The query sequence is considered to have a low similarity with a profile sequence when the p value is greater than 10⁻². Confidence in the degree of similarity between the query sequence and the profile sequence decreases as the p values become larger.

Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, 90 residues; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

It is apparent, when studying protein sequence families, that some regions have been better conserved than others during evolution. These regions are generally important for the function of a protein and/or for the maintenance of its three-dimensional structure. By analyzing the constant and variable properties of such groups of similar sequences, it is possible to derive a signature for a protein family or domain, which distinguishes its members from all other unrelated proteins. A pertinent analogy is the use of fingerprints by the police for identification purposes. A fingerprint is generally sufficient to identify a given individual. Similarly, a protein signature can be used to assign a new sequence to a specific family of proteins and thus to formulate hypotheses about its function. The PROSITE database is a compendium of such fingerprints (motifs) and may be used with search software such as Wisconsin GCG Motifs to find motifs or fingerprints in query sequences. PROSITE currently contains signatures specific for about a thousand protein families or domains. Each of these signatures comes with documentation providing background information on the structure and function of these proteins (Hofmann et al. (1999) Nucleic Acids Res. 27:215-219; Bucher and Bairoch., A generalized profile syntax for biomolecular sequences motifs and its function in automatic sequence interpretation (In) ISMB-94; Proceedings 2nd International Conference on Intelligent Systems for Molecular Biology; Altman et al. Eds. (1994), pp 53-61, AAAI Press, Menlo Park).

Translations of the provided nucleic acids can be aligned with amino acid profiles that define either protein families or common motifs. Also, translations of the provided nucleic acids can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided nucleic acids or corresponding cDNA or genes.

Profiles can designed manually by (1) creating an MSA, which is an alignment of the amino acid sequence of members that belong to the family and (2) constructing a statistical representation of the alignment. Such methods are described, for example, in Birney et al., Nucl. Acid Res. (1996) 24(14): 2730-2739. MSAs of some protein families and motifs are available for downloading to a local server. For example, the PFAM database with MSAs of 547 different families and motifs, and the software (HMMER) to search the PFAM database may be downloaded from ftp:/fftp.genetics.wustl.edu/pub/eddy/pfam-4.4/ to allow secure searches on a local server. Pfam is a database of multiple alignments of protein domains or conserved protein regions., which represent evolutionary conserved structure that has implications for the proteins function (Sonnhammer et al. (1998) Nucl. Acid Res. 26:320-322; Bateman etal. (1999) Nucleic Acids Res. 27:260-262).

The 3D_ali databank (Pasarella, S. and Argos, P. (1992) Prot. Engineering 5:121-137) was constructed to incorporate new protein structural and sequence data. The databank has proved useful in many research fields such as protein sequence and structure analysis and comparison, protein folding, engineering and design and evolution. The collection enhances present protein structural knowledge by merging information from proteins of similar main-chain fold with homologous primary structures taken from large databases of all known sequences. 3D_ali databank files may be downloaded to a secure local server from http://www.emblheidelberg.de/argos/ali/ali_form.html.

The identify and function of the gene that correlates to a nucleic acid described herein can be determined by screening the nucleic acids or their corresponding amino acid sequences against profiles of protein families. Such profiles focus on common structural motifs among proteins of each family. Publicly available profiles are known in the art.

In comparing a novel nucleic acid with known sequences, several alignment tools are available. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:443. GAP is best suited for global alignment of sequences. A third method, BestFit, functions by inserting gaps to maximize the number of matches using the local homology algorithm of Smith et al. (1981) Adv. Appl. Math. 2:482.

### IDENTIFICATION OF SECRETED & MEMBRANE-BOUND POLYPEPTIDES

Secreted and membrane-bound polypeptides of the present invention are of interest. Because both secreted and membrane-bound polypeptides comprise a fragment of contiguous hydrophobic amino acids, hydrophobicity predicting algorithms can be used to identify such polypeptides. A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 190: 207-219.

Another method of identifying secreted and membrane-bound polypeptides is to translate the nucleic acids of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine.

### IDENTIFICATION OF THE FUNCTION OF AN EXPRESSION PRODUCT

The biological function of the encoded gene product of the invention may be determined by empirical or deductive methods. One promising avenue, termed phylogenomics, exploits the use of evolutionary information to facilitate assignment of gene function. The approach is based on the idea that functional predictions can be greatly improved by focusing on how genes became similar in sequence during evolution instead of focusing on the sequence similarity itself. One of the major efficiencies that has emerged from plant genome research to date is that a large percentage of higher plant genes can be assigned some degree of function by comparing them with the sequences of genes of known function.

Alternatively, “reverse genetics” is used to identify gene function. Large collections of insertion mutants are available for Arabidopsis, maize, petunia, and snapdragon. These collections can be screened for an insertional inactivation of any gene by using the polymerase chain reaction (PCR) primed with oligonucleotides based on the sequences of the target gene and the insertional mutagen. The presence of an insertion in the target gene is indicated by the presence of a PCR product. By multiplexing DNA samples, hundreds of thousands of lines can be screened and the corresponding mutant plants can be identified with relatively small effort. Analysis of the phenotype and other properties of the corresponding mutant will provide an insight into the function of the gene.

In one method of the invention, the gene function in a transgenic Arabidopsis plant is assessed with anti-sense constructs. A high degree of gene duplication is apparent in Arabidopsis, and many of the gene duplications in Arabidopsis are very tightly linked. Large numbers of transgenic Arabidopsis plants can be generated by infecting flowers with Agrobacterium tumefaciens containing an insertional mutagen, a method of gene silencing based on producing double-stranded RNA from bidirectional transcription of genes in transgenic plants can be broadly useful for high-throughput gene inactivation (Clough and Bent (1999) Plant J. 17; Waterhouse et al. (1998) Proc. Natl. Acad. Sci. U.S.A. 95:13959). This method may use promoters that are expressed in only a few cell types or at a particular developmental stage or in response to an external stimulus. This could significantly obviate problems associated with the lethality of some mutations.

Virus-induced gene silencing may also find use for suppressing gene function. This method exploits the fact that some or all plants have a surveillance system that can specifically recognize viral nucleic acids and mount a sequence-specific suppression of viral RNA accumulation. By inoculating plants with a recombinant virus containing part of a plant gene, it is possible to rapidly silence the endogenous plant gene.

Antisense nucleic acids are designed to specifically bind to RNA, resulting in the formation of RNA-DNA or RNA-RNA hybrids, with an arrest of DNA replication, reverse transcription or messenger RNA translation. Antisense nucleic acids based on a selected nucleic acid sequence can interfere with expression of the corresponding gene. Antisense nucleic acids are typically generated within the cell by expression from antisense constructs that contain the antisense strand as the transcribed strand. Antisense nucleic acids based on the disclosed nucleic acids will bind and/or interfere with the translation of mRNA comprising a sequence complementary to the antisense nucleic acid. The expression products of control cells and cells treated with the antisense construct are compared to detect the protein product of the gene corresponding to the nucleic acid upon which the antisense construct is based. The protein is isolated and identified using routine biochemical methods.

As an alternative method for identifying function of the gene corresponding to a nucleic acid disclosed herein, dominant negative mutations are readily generated for corresponding proteins that are active as homomultimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants (see for example, Herskowitz (1987) Nature 329:219). Such techniques can be used to create loss of function mutations, which are useful for determining protein function.

Another approach for discovering the function of genes utilizes gene chips and microarrays. DNA sequences representing all the genes in an organism can be placed on miniature solid supports and used as hybridization substrates to quantitate the expression of all the genes represented in a complex mRNA sample. This information is used to provide extensive databases of quantitative information about the degree to which each gene responds to pathogens, pests, drought, cold, salt, photoperiod, and other environmental variation. Similarly, one obtains extensive information about which genes respond to changes in developmental processes such as germination and flowering. One can therefore determine which genes respond to the phytohormones, growth regulators, safeners, herbicides, and related agrichemicals. These databases of gene expression information provide insights into the “pathways” of genes that control complex responses. The accumulation of DNA microarray or gene chip data from many different experiments creates a powerful opportunity to assign functional information to genes of otherwise unknown function. The conceptual basis of the approach is that genes that contribute to the same biological process will exhibit similar patterns of expression. Thus, by clustering genes based on the similarity of their relative levels of expression in response to diverse stimuli or developmental or environmental conditions, it is possible to assign functions to many genes based on the known function of other genes in the cluster.

### CONSTRUCTION OF POLYPEPTIDES OF THE INVENTION AND VARIANTS THEREOF

The polypeptides of the invention include those encoded by the disclosed nucleic acids. These polypeptides can also be encoded by nucleic acids that, by virtue of the degeneracy of the genetic code, are not identical in sequence to the disclosed nucleic acids. Thus, the invention includes within its scope a polypeptide encoded by a nucleic acid having the sequence of any one of SEQ ID NOS: 1-999 or a variant thereof.

In general, the term “polypeptide” as used herein refers to both the full length polypeptide encoded by the recited nucleic acid, the polypeptide encoded by the gene represented by the recited nucleic acid, as well as portions or fragments thereof. “Polypeptides” also includes variants of the naturally occurring proteins, where such variants are homologous or substantially similar to the naturally occurring protein, and can be of an origin of the same or different species as the naturally occurring protein. In general, variant polypeptides have a sequence that has at least about 80%, usually at least about 90%, and more usually at least about 98% sequence identity with a differentially expressed polypeptide of the invention, as measured by BLAST using the parameters described above. The variant polypeptides can be naturally or non-naturally glycosylated, i.e., the polypeptide has a glycosylation pattern that differs from the glycosylation pattern found in the corresponding naturally occurring protein.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted.

Variants also include fragments of the polypeptides disclosed herein, particularly biologically active fragments and/or fragments corresponding to functional domains. Fragments of interest will typically be at least about 10 amino acids (aa) to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a nucleic acid having a sequence of any SEQ ID NOS:1-999, or a homolog thereof.

The protein variants described herein are encoded by nucleic acids that are within the scope of the invention. The genetic code can be used to select the appropriate codons to construct the corresponding variants.

### LIBRARIES AND ARRAYS

In general, a library of biopolymers is a collection of sequence information, which information is provided in either biochemical form (e.g., as a collection of nucleic acid or polypeptide molecules), or in electronic form (e.g., as a collection of genetic sequences stored in a computer-readable form, as in a computer system and/or as part of a computer program). The term biopolymer, as used herein, is intended to refer to polypeptides, nucleic acids, and derivatives thereof, which molecules are characterized by the possession of genetic sequences either corresponding to, or encoded by, the sequences set forth in the provided sequence list (seqlist). The sequence information can be used in a variety of ways, e.g., as a resource for gene discovery, as a representation of sequences expressed in a selected cell type, e.g. cell type markers, etc.

The nucleic acid libraries of the subject invention include sequence information of a plurality of nucleic acid sequences, where at least one of the nucleic acids has a sequence of any of SEQ ID NOS:1-999. By plurality is meant one or more, usually at least 2 and can include up to all of SEQ ID NOS:1-999. The length and number of nucleic acids in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the sequences or a subset thereof in a form that can be examined by means not directly applicable to the sequence as it exists in a nucleic acid. For example, the nucleotide sequence of the present invention, e.g. the nucleic acid sequences of any of the nucleic acids of SEQ ID NOS:1-999, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. Recorded refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.)

By providing the nucleotide sequence in computer readable form, the information can be accessed for a variety of purposes. Computer software to access sequence information is publicly available. For example, the BLAST (Altschul et al., supra.) and BLAZE (Brutlag et al. Comp. Chem. (1993) 17:203) search algorithms on a Sybase system can be used identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif with the stored sequence information. Search means are used to identify fragments or regions of the genome that match a particular target sequence or target motif. A variety of known algorithms are publicly known and commercially available, e.g. MacPattern (EMBL), BLASTN, BLASTX (NCBI) and tBLASTX. A “target sequence” can be any DNA or amino acid sequence of six or more nucleotides or two or more amino acids, preferably from about 10 to 100 amino acids or from about 30 to 300 nucleotide residues.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but arc not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks fragments of the genome possessing varying degrees of homology to a target sequence or target motif. Such presentation provides a skilled artisan with a ranking of sequences and identifies the degree of sequence similarity contained in the identified fragment.

A variety of comparing means can be used to compare a target sequence or target motif with the data storage means to identify sequence fragments of the genome. A skilled artisan can readily recognize that any one of the publicly available homology search programs can be used as the search means for the computer based systems of the present invention.

As discussed above, the “library” of the invention also encompasses biochemical libraries of the nucleic acids of SEQ ID NOS:1-999, e.g., collections of nucleic acids representing the provided nucleic acids. The biochemical libraries can take a variety of forms, e.g. a solution of cDNAs, a pattern of probe nucleic acids stably bound to a surface of a solid support (microarray) and the like. By array is meant an article of manufacture that has a solid support or substrate with one or more nucleic acid targets on one of its surfaces, where the number of distinct nucleic may be in the hundreds, thousand, or tens of thousands. Each nucleic acid will comprise at 18 nt and often at least 25 nt, and often at least 100 to 1000 nucleotides, and may represent up to a complete coding sequence or cDNA. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the where the polypeptides of the library will represent at least a portion of the polypeptides encoded by SEQ ID NOS:1-999.

### GENETICALLY ALTERED CELLS AND TRANSGENICS

The subject nucleic acids can be used to create genetically modified and transgenic organisms, usually plant cells and plants, which may be monocots or dicots. The term transgenic, as used herein, is defined as an organism into which an exogenous nucleic acid construct has been introduced, generally the exogenous sequences are stably maintained in the genome of the organism. Of particular interest are transgenic organisms where the genomic sequence of germ line cells has been stably altered by introduction of an exogenous construct.

Typically, the transgenic organism is altered in the genetic expression of the introduced nucleotide sequences as compared to the wild-type, or unaltered organism. For example, constructs that provide for over-expression of a targeted sequence, sometimes referred to as a “knock-in”, provide for increased levels of the gene product. Alternatively, expression of the targeted sequence can be down-regulated or substantially eliminated by introduction of a “knock-out” construct, which may direct transcription of an anti-sense RNA that blocks expression of the naturally occurring mRNA, by deletion of the genomic copy of the targeted sequence, etc.

In one method, large numbers of genes are simultaneously introduced in order to explore the genetic basis of complex traits, for example by making plant artificial chromosome (PLAC) libraries. The centromeres in Arabidopsis have been mapped and current genome sequencing efforts will extend through these regions. Because Arabidopsis telomeres are very similar to those in yeast one may use a hybrid sequence of alternating plant and yeast sequences that function in both types of organisms, developing yeast artificial chromosome-PLAC libraries, and then introducing them into a suitable plant host to evaluate the phenotypic consequences. By providing a defined chromosomal environment for cloned genes, the use of PLACs may also enhance the ability to produce transgenic plants with defined levels of gene expression.

It has been found in many organisms that there is significant redundancy in the representation of genes in a genome. That is, a particular gene function is likely by represented by multiple copies of similar coding sequences in the genome. These copies are typically conserved in the amino acid sequence, but may diverge in the sequence of non-translated sequences, and in their codon usage. In order to knock out a particular genetic function in an organism, it may not be sufficient to delete a genomic copy of a single gene. In such cases it may be preferable to achieve a genetic knock-out with an anti-sense construct, particularly where the sequence is aligned with the coding portion of the mRNA.

Methods of transforming plant cells are well-known in the art, and include protoplast transformation, tungsten whiskers (Coffee et al., U.S. Pat. No. 5,302,523, issued Apr. 12, 1994), directly by microorganisms with infectious plasmids, use of transposons (U.S. Pat. No. 5,792,294), infectious viruses, the use of liposomes, microinjection by mechanical or laser beam methods, by whole chromosomes or chromosome fragments, electroporation, silicon carbide fibers, and microprojectile bombardment.

For example, one may utilize the biolistic bombardment of meristem tissue, at a very early stage of development, and the selective enhancement of transgenic sectors toward genetic homogeneity, in cell layers that contribute to germline transmission. Biolistics-mediated production of fertile, transgenic maize is described in Gordon-Kamm et al. (1990), Plant Cell 2:603; Fromm et al. (1990) Bio/Technology 8: 833, for example. Alternatively, one may use a microorganism, including but not limited to, Agrobacterium tumefaciens as a vector for transforming the cells, particularly where the targeted plant is a dicotyledonous species. See, for example, U.S. Pat. No. 5,635,381. Leung et al. (1990) Curr. Genet. 17(5):409-11 describe integrative transformation of three fertile hermaphroditic strains of Arabidopsis thaliana using plasmids and cosmids that contain an E. coli gene linked to Aspergillus nidulans regulatory sequences.

Preferred expression cassettes for cereals may include promoters that are known to express exogenous DNAs in corn cells. For example, the Adhl promoter has been shown to be strongly expressed in callus tissue, root tips, and developing kernels in corn. Promoters that are used to express genes in corn include, but are not limited to, a plant promoter such as the, CaMV 35S promoter (Odell et al., Nature, 313, 810 (1985)), or others such as CaMV 19S (Lawton et al., Plant Mol. Biol., 9, 31F (1987)), nos (Ebert et al., PNAS USA, 84, 5745 (1987)), Adh (Walker et al., PNAS USA, 84, 6624 (1987)), sucrose synthase (Yang et al., PNAS USA, 87, 4144 (1990)), .alpha.-tubulin, ubiquitin, actin (Wang et al., Mol. Cell. Biol., 12, 3399 (1992)), cab (Sullivan et al., Mol. Gen. Genet, 215, 431 (1989)), PEPCase (Hudspeth et al., Plant Mol. Biol., 12, 579 (1989)), or those associated with the R gene complex (Chandler et al., The Plant Cell, 1, 1175 (1989)). Other promoters useful in the practice of the invention are known to those of skill in the art.

Tissue-specific promoters, including but not limited to, root-cell promoters (Conkling et al., Plant Physiol., 93, 1203 (1990)), and tissue-specific enhancers (Fromm et al., The Plant Cell, 1, 977 (1989)) are also contemplated to be particularly useful, as are inducible promoters such as water-stress-, ABA- and turgor-inducible promoters (Guerrero et al., Plant Molecular Biology, 15, 11-26)), and the like.

Regulating and/or limiting the expression in specific tissues may be functionally accomplished by introducing a constitutively expressed gene (all tissues) in combination with an antisense gene that is expressed only in those tissues where the gene product is not desired. Expression of an antisense transcript of this preselected DNA segment in an rice grain, using, for example, a zein promoter, would prevent accumulation of the gene product in seed. Hence the protein encoded by the preselected DNA would be present in all tissues except the kernel.

Alternatively, one may wish to obtain novel tissue-specific promoter sequences for use in accordance with the present invention. To achieve this, one may first isolate cDNA clones from the tissue concerned and identify those clones which are expressed specifically in that tissue, for example, using Northern blotting or DNA microarrays. Ideally, one would like to identify a gene that is not present in a high copy number, but which gene product is relatively abundant in specific tissues. The promoter and control elements of corresponding genomic clones may then be localized using the techniques of molecular biology known to those of skill in the art. Alternatively, promoter elements can be identified using enhancer traps based on T-DNA and/or transposon vector systems (see, for example, Campisi et al. (1999) Plant J. 17:699-707; Gu et al. (1998) Development 125:1509-1517).

In some embodiments of the present invention expression of a DNA segment in a transgenic plant will occur only in a certain time period during the development of the plant. Developmental timing is frequently correlated with tissue specific gene expression. For example, in corn expression of zein storage proteins is initiated in the endosperm about 15 days after pollination.

Ultimately, the most desirable DNA segments for introduction into a plant genome may be homologous genes or gene families which encode a desired trait (e.g., increased disease resistance) and which are introduced under the control of novel promoters or enhancers, etc., or perhaps even homologous or tissue-specific (e.g., root-, grain- or leaf-specific) promoters or control elements.

The genetically modified cells are screened for the presence of the introduced genetic material. The cells may be used in functional studies, drug screening, etc., e.g. to study chemical mode of action, to determine the effect of a candidate agent on pathogen growth, infection of plant cells, etc.

The modified cells are useful in the study of genetic function and regulation, for alteration of the cellular metabolism, and for screening compounds that may affect the biological function of the gene or gene product. For example, a series of small deletions and/or substitutions may be made in the hosts native gene to determine the role of different domains and motifs in the biological function. Specific constructs of interest include anti-sense, as previously described, which will reduce or abolish expression, expression of dominant negative mutations, and over-expression of genes.

Where a sequence is introduced, the introduced sequence may be either a complete or partial sequence of a gene native to the host, or may be a complete or partial sequence that is exogenous to the host organism, e.g., an A. thaliana sequence inserted into wheat plants. A detectable marker, such as aldA, lac Z, etc. may be introduced into the locus of interest, where upregulation of expression will result in an easily detected change in phenotype.

One may also provide for expression of the gene or variants thereof in cells or tissues where it is not normally expressed, at levels not normally present in such cells or tissues, or at abnormal times of development, during sporulation, etc. By providing expression of the protein in cells in which it is not normally produced, one can induce changes in cell behavior.

DNA constructs for homologous recombination will comprise at least a portion of the provided gene or of a gene native to the species of the host organism, wherein the gene has the desired genetic modification(s), and includes regions of homology to the target locus (see Kempin et al. (1997) Nature 389:802-803). DNA constructs for random integration or episomal maintenance need not include regions of homology to mediate recombination. Conveniently, markers for positive and negative selection are included. Methods for generating cells having targeted gene modifications through homologous recombination are known in the art.

Embodiments of the invention provide processes for enhancing or inhibiting synthesis of a protein in a plant by introducing a provided nucleic acids sequence into a plant cell, where the nucleic acid comprises sequences encoding a protein of interest. For example, enhanced resistance to pathogens may be achieved by inserting a nucleic acid encoding an activator in a vector downstream from a promoter sequence capable of driving constitutive high-level expression in a plant cell. When grown into plants, the transgenic plants exhibit increased synthesis of resistance proteins, and increased resistance to pathogens.

Other embodiments of the invention provide processes for enhancing or inhibiting synthesis of a tolerance factor in a plant by introducing a nucleic acid of the invention into a plant cell, where the nucleic acid comprises sequences encoding a tolerance factor. For example, enhanced tolerance to an environmental stress may be achieved by inserting a nucleic acid encoding an activator in a vector downstream from a promoter sequence capable of driving constitutive high-level expression in a plant cell. When grown into plants, the transgenic plants exhibit increased synthesis of tolerance proteins, and increased tolerance to environmental stress.

Factors which are involved, directly or indirectly in biosynthetic pathways whose products are of commercial, nutritional, or medicinal value include any factor, usually a protein or peptide, which regulates such a biosynthetic pathway (e.g., an activator or repressor); which is an intermediate in such a biosynthetic pathway; or which is a product that increases the nutritional value of a food product; a medicinal product; or any product of commercial value and/or research interest. Plant and other cells may be genetically modified to enhance a trait of interest, by upregulating or down-regulating factors in a biosynthetic pathway.

### SCREENING ASSAYS

The polypeptides encoded by the provided nucleic acid sequences, and cells genetically altered to express such sequences, are useful in a variety of screening assays to determine effect of candidate inhibitors, activators., or modifiers of the gene product. One may determine what insecticides, fungicides and the like have an enhancing or synergistic activity with a gene. Alternatively, one may screen for compounds that mimic the activity of the protein. Similarly, the effect of activating agents may be used to screen for compounds that mimic or enhance the activation of proteins. Candidate inhibitors of a particular gene product are screened by detecting decreased from the targeted gene product.

The screening assays may use purified target macromolecules to screen large compound libraries for inhibitory drugs; or the purified target molecule may be used for a rational drug design program, which requires first determining the structure of the macromolecular target or the structure of the macromolecular target in association with its customary substrate or ligand. This information is then used to design compounds which must be synthesized and tested further. Test results are used to refine the molecular models and drug design process in an iterative fashion until a lead compound emerges.

Drug screening may be performed using an in vitro model, a genetically altered cell, or purified protein. One can identify ligands or substrates that bind to, modulate or mimic the action of the target genetic sequence or its product. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions.

Where the nucleic acid encodes a factor involved in a biosynthetic pathway, as described above, it may be desirable to identify factors, e.g., protein factors, which interact with such factors. One can identify interacting factors, ligands, substrates that bind to, modulate or mimic the action of the target genetic sequence or its product. A wide variety of assays may be used for this purpose, including labeled in vitro protein-protein binding assays, electrophoretic mobility shift assays, immunoassays for protein binding, and the like. In vivo assays for protein-protein interactions in E. coli and yeast cells are also well-established (see Hu et al. (2000) Methods 20:80-94; and Bai and Elledge (1997) Methods Enzymol. 283:141-156).

The purified protein may also be used for determination of three-dimensional crystal structure, which can be used for modeling intermolecular interactions. It may also be of interest to identify agents that modulate the interaction of a factor identified as described above with a factor encoded by a nucleic acid of the invention. Drug screening can be performed to identify such agents. For example, a labeled in vitro protein-protein binding assay can be used, which is conducted in the presence and absence of an agent being tested.

The term “agent” as used herein describes any molecule, e.g. protein or pharmaceutical, with the capability of altering or mimicking a physiological function. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and organism extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The compounds having the desired biological activity may be administered in an acceptable carrier to a host. The active agents may be administered in a variety of ways. Depending upon the manner of introduction, the compounds may be formulated in a variety of ways. The concentration of therapeutically active compound in the formulation may vary from about 0.01-100 wt. %.

It must be noted that as used herein and in the appended claims, the singular forms “a”, “and”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the methods and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees Celsius, and pressure is at or near atmospheric.

### EXPERIMENTAL

### Cloning and Characterization of Arabidopsis thaliana Genes.

Following DNA isolation, sequencing was performed using the Dye Primer Sequencing protocol, below. The sequencing reactions were loaded by hand onto a 48 lane ABI 377 and run on a 36 cm gel with the 36E-2400 run module and extraction. Gel analysis was performed with ABI software.

The Phred program was used to read the sequence trace from the ABI sequencer, call the bases and produce a sequence read and a quality score for each base call in the sequence., (Ewing et al. (1998) Genome Research 8:175-185; Ewing and Green (1998) Genome Research 8:186-194.) PolyPhred may be used to detect single nucleotide polymorphisms in sequences (Kwok et al. (1994) Genomics 25:615-622; Nickerson et al. (1997) Nucleic Acids Research 25(14):2745-2751.)

MicroWave Plasmid Protocol: Fill Beckman 96 deep-well growth blocks with 1 ml of TB containing 50 μg of ampicillin per ml. Inoculate each well with a colony picked with a toothpick or a 96-pin tool from a glycerol stock plate. Cover the blocks with a plastic lid and tape at two ends to hold lid in place. Incubate overnight (16-24 hours depending on the host stain) at 37° C. with shaking at 275 rpm in a New Brunswick platform shaker. Pellet cells by centrifugation for 20 minutes at 3250 rpm in a Beckman GS-R6K, decant TB and freeze pelleted cell in the 96 well block. Thaw blocks on the bench when ready to continue.

Prepare the MW-Tween20 solution

| For four blocks: | | | | For 16 blocks: |
|---|---|---|---|---|
| 50 | ml STET/TWEEN20 | | | 200 ml STET/TWEEN |
| 2 | tubes RNAse | (10 | mg/ml, 600 ulea) | 8 tubes RNAse |
| 1 | tube lysozyme | (25 | mg) | 4 tubes lysozyme |

Pipette RNAse and Lysozyme into the corner of a beaker. Add Tween 20 solution and swirl to mix completely. Use the Multidrop (or Biohit) to add 25 ul of sterile H₂O (from the L size autoclaved bottles) to each well. Resuspend the pellets by vortexing on setting 10 of the platform vortexer. Check pellets after 4 min. and repeat as necessary to resuspend completely. Use the multidrop to add 70 μl of the freshly prepared MW-Tween 20 solution to each well. Vortex at setting 6 on the platform vortex for 15 seconds. Do not cause frothing.

Incubate the blocks at room temperature for 5 min. Place two blocks at a time in the microwave (1000 Wafts) with the tape (placed on the H1 to H12 side of the block) facing away from each other and turn on at full power for 30 seconds. Rotate the blocks so that the tapes face towards each other and turn on at full power again for 30 seconds.

Immediately remove the blocks from the microwave and add 300 μl of sterile ice cold H₂O with the Multidrop. Seal the blocks with foil tape and place them in an H₂O/ice bath.

Vortex the blocks on 5 for 15 seconds and leave them in the H₂O/Ice bath. Return to step 7 until all the blocks are in the ice water bath. Incubate the blocks for 15 minutes on ice. Spin the blocks for 30 minutes in the Beckman GS-6KR with GH3.8 rotor with Microplus carrier at 3250 rpm.

Transfer 100 μl of the supernatant to Corning/Costar round bottom 96 well trays. Cover with foil and put into fridge if to be sequenced right away. If not to be sequenced in the next day, freeze them at −20° C.

Dye Primer Sequencing: Spin down the DP brew trays and DNA template by pulsing in the Beckman GS-6KR with GH3.8 rotor with Microplus carrier. Big Dye Primer reaction mix trays (one 96 well cycleplate (Robbins) for each nucleotide), 3 microliters of reaction mix per well.

Use twelve channel pipetter (Costar) to add 2 μl of template to one each G,A,T,C, trays for each template plate. Pulse again to get both the reaction mix and template into the bottom of the cycle plate and put them into the MJ Research DNA Tetrad (PTC-225).

Start program Dye-Primer. Dye-primer is:

96° C., 1 min 1 cycle

96° C., 10 sec.

55° C., 5 sec.

70° C., 1 min 15 cycles

96° C., 10 sec.

70° C., 1 min. 15 cycles

4° C. soak

When done cycling, using the Robbins Hydra 290 add 100 μl of 100% ethanol to the A reaction cycle plate and pool the contents of all four cycle plates into the appropriate well.

To perform ethanol precipitation: Use Hydra program 4 to add 100 μl 100% ethanol to each A tray. Use Hydra program 5 to transfer the ethanol and therefore combine the samples from plate to plate. Once the G, A, T, and C trays of each block are mixed, spin for 30 minutes at 3250 in the Beckman. Pour off the ethanol with a firm shake and blot on a paper towel before drying in the speed vac (˜10 minutes or until dry). If ready to load add 3 μl dye and denature in the oven at 95° C. for ˜5 minutes and load 2 μl. If to store, cover with tape and store at −20° C.

Common Solutions

Terrific Broth

Per liter:

900 ml H₂O

12 g bacto tryptone

24 g bacto-yeast extract

4 ml glycerol

Shake until dissolved and then autoclave. Allow the solution to cool to 60° C. or less and then add 100 ml of sterile 0.17M KH₂PO₄, 0.72M K₂HPO₄ (in the hood w/sterile technique).

0.17M KH₂PO₄, 0.72M K₂HPO₄

Dissolve 2.31 g of KH₂PO₄ and 12.54 g of K₂HPO₄ in 90 ml of H₂O.

Adjust volume to 100 ml with H₂O and autoclave.

Sequence loading Dye

20 ml deionized formamide

3.6 ml dH₂O

400 pi 0.5M EDTA, pH 8.0

0.2 g Blue Dextran

*Light sensitive, cover in foil or store in the dark.

STET/TWEEN

10 ml 5M NaCl

5 ml 1 M Tris, pH 8.0

1 ml 0.5M EDTA., pH 8.0

25 ml Tween20

Bring volume to 500 ml with H₂O

The sequencing reactions are run on an ABI 377 sequencer per manufacturer's' instructions. The sequencing information obtained each run are analyzed as follows.

Sequencing reads are screened for ribosomal., mitochondrial., chloroplast or human sequence contamination.. In good sequences, vector is marked by x's. These sequences go into biolims regardless of whether or not they pass the criteria for a ‘good’ sequence. This criteria is >=100 bases with phred score of >=20 and 15 of these bases adjacent to each other.

Sequencing reads that pass the criteria for good sequences are downloaded for assembly into consensus sequences (contigs). The program Phrap (copyrighted by Phil Green at University of Washington, Seattle, Wash.) utilizes both the Phred sequence information and the quality calls to assemble the sequencing reads. Parameters used with Phrap were determined empirically to minimize assembly of chimeric sequences and maximize differential detection of closely related members of gene families. The following parameters were used with the Phrap program to perform the assembly:

| | | |
|---|---|---|
| Penalty | −6 | Penalty for mismatches(substitutions) |
| Minmatch | 40 | Minimum length of matching sequence to use in |
| | | assembly of reads |
| Trim penalty | 0 | penalty used for identifying degenerate sequence at |
| | | beginning and end of read. |
| Minscore | 80 | Minimum alignment score |

Results from the Phrap analysis yield either contigs consisting of a consensus of two or more overlapping sequence reads, or singlets that are non-overlapping.

The contig and singlets assembly were further analyzed to eliminate low quality sequence utilizing a program to filter sequences based on quality scores generated by the Phred program. The threshold quality for “high quality” base calls is 20. Sequences with less than 50 contiguous high quality bases calls at the beginning of the sequence, and also at the end of the sequence were discarded. Additionally, the maximum allowable percentage of “low quality base calls in the final sequence is 2%, otherwise the sequence is discarded.

The stand-alone BLAST programs and Genbank databases were downloaded from NCBI for use on secure servers at the Paradigm Genetics, Inc. site. The sequences from the assembly were compared to the GenBank NR database downloaded from NCBI using the gapped version (2.0) of BLASTX. BLASTX translates the DNA sequence in all six reading frames and compares it to an amino acid database. Low complexity sequences are filtered in the query sequence. (Altschul et al. (1997) Nucleic Acids Res 25(17):3389-402).

Genbank sequences found in the BLASTX search with an E Value of less than 1e⁻¹⁰ are considered to be highly similar, and the Genbank definition lines were used to annotate the query sequences.

When no significantly similar sequences were found as a result of the BLASTX search, the query sequences were compared with the PROSITE database (Bairoch, A. (1992) PROSITE: A dictionary of sites and patterns in proteins. Nucleic Acids Research 20:2013-2018. ) to locate functional motifs.

Query sequences were first translated in six reading frames using the Wisconsin GCG pepdata program (Wisconsin Package Version 10.0, Genetics Computer Group (GCG) , Madison, Wis., USA. ). The Wisconsin GCG motifs program (Wisconsin Package Version 10.0, Genetics Computer Group (GCG), Madison, Wis., USA.) was used to locate motifs in the peptide sequence, with no mismatches allowed. Motif names from the PROSITE results were used to annotate these query sequences.

TABLE I

| SEQ | | |
|---|---|---|
| ID | Reference | Annotation |
| 1 | 2024001 | 1E-106 >gb\|AAD34616.1\|AF153284_1 (AF153284) |
| | | progesterone-binding protein homolog [Arabidopsis |
| | | thaliana] Length = 220 |
| 2 | 2024002 | Tyr_Phospho Site(56-63) |
| 3 | 2024003 | 5E-88 >pir\|\|A54809 disease resistance protein |
| | | RPS2 - Arabidopsis thaliana >gi\|548086 (U14158) |
| | | RPS2 [Arabidopsis thaliana] >gi\|549979 (U12860) |
| | | RPS2 Arabidopsis thali- |
| | | ana]>gi\|4538938\|emb\|CAB39674.1\| (AL049483) |
| | | disease |
| 4 | 2024004 | 7E-81 ) >sp\|P25069\|CAL2_ARATH CALMO- |
| | | DULIN-2/3/5 >gi\|99671\|pir\|\|S22503 calmodulin - |
| | | Arabidopsis thaliana >gi\|1076437\|pir\|\|S53006 |
| | | calmodulin - leaf mustard >gi\|2146726\|pir\|\|S71513 |
| | | calmodulin - Arabidopsis thaliana >gi\|166651 |
| | | (M38380) calmodulin-2 Arabidopsis thali- |
| | | ana] >gi\|166653 (M73711) calmodulin-3 [Arabidopsis |
| | | thaliana] >gi\|474183\|emb\|CAA47690\| |
| | | (X67273) calmodulin Arabidopsis thali- |
| | | ana] >gi\|497992 (U10150) calmodulin [Brassica |
| | | napus] >gi\|899058 (M88307) calmodulin [Brassica |
| | | juncea] >gi\|1183005\|dbj\|BAA082831 (D45848) |
| | | calmodulin [Arabidopsis thaliana] >gi\|3402706 |
| | | (AC004261) unknown protein Arabidopsis thali- |
| | | ana] >gi\|3885333 (AC005623) calmodulin |
| | | [Arabidopsis thaliana] >gi\|228407\|prf\|\|1803520A |
| | | calmodulin 2 [Arabidopsis thaliana] Length = 149 |
| 5 | 2024005 | 1E-116 >pir\|\|S28426 ubiquitin precursor - |
| | | wild oat >gi\|15989\|emb\|CAA49200\| (X69422) |
| | | tetraubiquitin [Avena fatua] >gi\|777758 |
| | | (L41658) polyubiguitin [Saccharum sp.] Length = 305 |
| 6 | 2024006 | 6E-77 >sp\|P42699\|D112_ARATH DNA- |
| | | DAMAGE-REPAIR/TOLERATION PROTEIN |
| | | DRT112 PRECURSOR >gi\|421830\|pir\|\|S33707 |
| | | DRT112 protein - Arabidopsis thaliana >gi\|166696 |
| | | (M98456) DRT112 [Arabidopsis thaliana] |
| | | Length = 167 |
| 7 | 2024007 | Zinc_Finger_C3hc4(1162-1171) |
| 8 | 2024008 | Rgd(1473-1475) |
| 9 | 2024009 | 4E-65 >dbj\|BAA84370.1\| (AP000423) |
| | | ATPase alpha subunit [Arabidopsis thaliana] |
| | | Length = 507 |
| 10 | 2024010 | Tyr_Phospho_Site(658-664) |
| 11 | 2024011 | Tyr_Phospho_Site(1191-1198) |
| 12 | 2024012 | Tyr_Phospho_Site(1122-1128) |
| 13 | 2024013 | IE-137 >dbj\|BAA84375.1\| (AP000423) |
| | | RNA polymerase beta' subunit-2 |
| | | [Arabidopsis thaliana] Length = 1376 |
| 14 | 2024014 | Tyr_Phospho_Site(1352-1360) |
| 15 | 2024015 | 4E-98 >gb\|AAD15512\| (AC006439) |
| | | Reri protein [Arabidopsis thaliana] Length = 566 |
| 16 | 2024016 | 1E-116 >emb\|CAB38949.1\| (AL049171) |
| | | 1-aminocyclopropane-1-carboxylate synthase-like |
| | | protein [Arabidopsis thaliana] Length = 447 |
| 17 | 2024017 | 2E-78 >sp\|P25070\|TCH2_ARATH CAL- |
| | | MODULIN-RELATED PROTEIN 2, TOUCH-IN- |
| | | DUCED >gi\|2583169 (AF026473) calmodulin-related |
| | | protein [Arabidopsis thaliana] Length = 161 |
| 18 | 2024018 | Tyr_Phospho_Site(193-199) |
| 19 | 2024019 | Tyr_Phospho_Site(879-886) |
| 20 | 2024020 | 7E-50 >emb\|CAA74401.1\| (Y14072) |
| | | HMG protein [Arabidopsis thaliana] |
| | | Length = 144 |
| 21 | 2024021 | Tyr_Phospho_Site(940-947) |
| 22 | 2024022 | 1E-110 >prf\|\|1804333C Gin synthetase [Arabidopsis |
| | | thaliana] Length = 430 |
| 23 | 2024023 | 2E-85 >sp\|P35133\|UBCA_ARATH UBIQUITIN- |
| | | CONJUGATING ENZYME E2-17 KD 10 |
| | | (UBIQUITIN-PROTEIN LIGASE 10) |
| | | (UBIQUITIN CARRIER PROTEIN |
| | | 10) >gi\|421858\|pir\|\|S32672 ubiquitin—protein |
| | | ligase (EC 6.3.2.19) UBC10 - Arabidopsis thali- |
| | | ana >gi\|297878\|emb\|CAA78715\| (Z14991) ubiquitin |
| | | conjugating enzyme [Arabidopsis thali- |
| | | ana] >gi\|349213 (L00640) ubiquitin conjugating |
| | | enzyme [Arabidopsis thaliana] Length = 148 |
| 24 | 2024024 | 3E-29 >sp\|Q96286\|DCAM_ARATH S-ADEN- |
| | | OSYLMETHIONINE DECARBOXYLASE |
| | | PROENZYME (ADOMETDC) |
| | | (SAMDC) >gi\|1531763\|emb\|CAA69073\| (Y07765) |
| | | s-adenosylmethionine decarboxylase |
| | | [Arabidopsis thaliana] Length = 366 |
| 25 | 2024025 | Tyr_Phospho_Site(561-569) |
| 26 | 2024026 | Tyr_Phospho_Site(1000-1007) |
| 27 | 2024027 | 3E-94 >emb\|CAB41856.1\| (AL049746) |
| | | ABC-type transport-like protein |
| | | [Arabidopsis thaliana] Length = 1011 |
| 28 | 2024028 | 1E-112 >sp\|P43082\|HEVL_ARATH HEVEIN- |
| | | LIKE PROTEIN PRECURSOR >gi\|407248 |
| | | (U01880) pre-hevein-like protein Arabidopsis thali- |
| | | ana] >gi\|6175186\|gb\|AAF04912.1\|AC011437_27 |
| | | (AC011437) hevein-like protein |
| | | precursor [Arabidopsis thaliana] |
| 29 | 2024029 | 8E-98 >gi\|3242705 (AC003040) |
| | | nicotinate phosphoribosyltransferase |
| | | [Arabidopsis thaliana] Length = 574 |
| 30 | 2024030 | Tyr_Phospho_Site(853-860) |
| 31 | 2024031 | 1E-115 >gi\|2281083 (AC002333) |
| | | polygalacturonase isolog [Arabidopsis |
| | | thaliana] Length = 384 |
| 32 | 2024032 | Pkc_Phospho_Site(27-29) |
| 33 | 2024033 | 4E-28 >sp\|P16970\|PMP7_RAT 70 |
| | | KD PEROXISOMAL MEMBRANE PROTEIN |
| | | (PMP70) >gi\|111319\|pir\|\|A35723 70K |
| | | peroxisomal membrane protein - |
| | | rat >gi\|220862\|dbj BAA14086\| (D90038) |
| | | PMP70 [Rattus norvegicus] Length = 659 |
| 34 | 2024034 | 2E-19 >gi\|3249088 (AC004473) |
| | | Contains similarity to goliath protein |
| | | gb\|M97204 from D. melanogster. [Arabidopsis |
| | | thaliana] Length = 327 |
| 35 | 2024035 | 2E-13 >pir\|fJN0673 ubiquitin-like |
| | | fusion protein An1a - African clawed frog |
| | | Length = 693 |
| 36 | 2024036 | Tyr_Phospho_Site(554-562) |
| 37 | 2024037 | 1E-10 >gb\|AAD25756.1\|AC007060_14 (AC007060) |
| | | Contains the PF\|00650 CRAL/TRIO phosphatidyl- |
| | | inositol-transfer protein domain. ESTs gb\|T76582, |
| | | gb\|N06574 and gb\|Z25700 come from this gene. |
| | | [Arabidopsis thaliana] Length = 540 |
| 38 | 2024038 | 1E-30 >gb\|AAD25756.1\|AC007060_14 (AC007060) |
| | | Contains the PF\|00650 CRAL/TRIO phosphatidyl- |
| | | inositol-transfer protein domain. ESTs gb\|T76582, |
| | | gb\|N06574 and gb\|Z25700 come from this gene. |
| | | [Arabidopsis thaliana] Length = 540 |
| 39 | 2024039 | IE-101 >gb\|AAD32870.1\|AC005489_8 (AC005489) |
| | | F14N23.8 [Arabidopsis thaliana] Length = 223 |
| 40 | 2024040 | 3E-34 >gb\|AAD17415\| (AC006248) |
| | | serine/threonine kinase [Arabidopsis |
| | | thaliana] Length = 365 |
| 41 | 2024041 | 1E-74 >sp\|Q08112\|RS15_ARATH 40S |
| | | RIBOSOMAL PROTEIN S15 >gi\|629556\|pir\|\|S43412 |
| | | ribosomal protein S15 - Arabidopsis thali- |
| | | ana >gi\|313152\|emb\|CAA80679\| (Z23161) ribosomal |
| | | protein S15 [Arabidopsis |
| | | thaliana] >gi\|313188\|emb\|CAA80681\| (Z23162) |
| | | ribosomal protein S15 [Arabidopsis |
| | | thaliana] >gi\|1903366\|gb\|AAB70449\| (AC000104) |
| | | Strong similarity to Oryza 40S ribosomal protein S15. |
| | | ESTs gb\|R29788,gb\|ATTS0365 come from this |
| | | gene. [Arabidopsis thaliana] Length = 152 |
| 42 | 2024042 | Tyr_Phospho_Site(1144-1151) |
| 43 | 2024043 | 2E-70 >sp\|Q05091\|PGIP_PYRCO POLY- |
| | | GALACTURONASE INHIBITOR PRECURSOR |
| | | (POLYGALACTURONASE-INHIBITING PROTEIN) |
| | | >gi\|543660\|pir\|\|JQ2262 Polygalacturonase inhibitor |
| | | precursor - Pyrus communis >gi\|169684 (L09264) |
| | | polygalacturonase inhibitor [Pyrus communis] |
| | | Length = 330 |
| 44 | 2024044 | 2E-73 >gi\|3201969 (AF068332) |
| | | submergence induced protein 2A [Oryza |
| | | sativa] Length = 198 |
| 45 | 2024045 | 1E-108 >emb\|CAB41167.1\| (AL049659) |
| | | cytochrome P450-like protein [Arabidopsis thaliana] |
| | | Length = 490 |
| 46 | 2024046 | 1E-92 >emb\|CAA66959\| (X98315) peroxidase |
| | | [Arabidopsis thaliana] >gi\|1429221\|emb\|CAA67313\| |
| | | (X98777) peroxidase ATP16a [Arabidopsis |
| | | thaliana] >gi\|4455802\|emb\|CAB37193\| (AJ133036) |
| | | peroxidase [Arabidopsis thaliana] Length = 352 |
| 47 | 2024047 | 4E-31 >sp\|P25070\|TCH2_ARATH CAL- |
| | | MODULIN-RELATED PROTEIN 2, TOUCH- |
| | | INDUCED >gi\|2583169 (AF026473) calmodulin-related |
| | | protein [Arabidopsis thaliana] Length = 161 |
| 48 | 2024048 | 2E-93 >sp\|Q43291\|RL2I_ARATH 60S |
| | | RIBOSOMAL PROTEIN L21 >gi\|2160162 |
| | | (AC000132) Similar to ribosomal protein L21 |
| | | (gb\|L38826). ESTs gb\|AA395597,gb\|ATTS5197 come |
| | | from this gene. [Arabidopsis thaliana] >gi\|3482935 |
| | | (AC003970) ribosomal protein L21 [Arabidopsis thaliana] |
| | | Length = 164 |
| 49 | 2024049 | Tyr_Phospho_Site(102-108) |
| 50 | 2024050 | Tyr_Phospho_Site(605-612) |
| 51 | 2024051 | 1E-116 ) >gi\|3885328 (AC005623) |
| | | serine/threonine protein kinase |
| | | [Arabidopsis thaliana] Length = 441 |
| 52 | 2024052 | Pkc_Phospho_Site(29-31) |
| 53 | 2024053 | Tyr_Phospho_Site(2-9) |
| 54 | 2024054 | 1E-90 ) >emb\|CAA10457.1\| (AJ131580) |
| | | glutathione transferase AtGST 10 |
| | | [Arabidopsis thaliana] Length = 245 |
| 55 | 2024055 | 2E-33 >ref\|NP_002003.1\|PFHIT\| fragile histidine |
| | | triad gene >gi\|1706794\|sp\|P49789\|FHIT_HUMAN |
| | | BIS(5′-ADENOSYL)-TRIPHOSPHATASE |
| | | (DIADENOSINE 5′, 5″″-P1,P3-TRIPHOSPHATE |
| | | HYDROLASE) (DINUCLEOSIDETRIPHOSPHA- |
| | | TASE) (AP3A HYDROLASE) (AP3AASE) |
| | | (FRAGILE HISTIDINE TRIAD |
| | | PROTEIN) >gi\|3114520\|pdb\| |
| 56 | 2024056 | Tyr_Phospho_Site(631-637) |
| 57 | 2024057 | Tyr_Phospho_Site(659-666) |
| 58 | 2024058 | Tyr_Phospho_Site(576-582) |
| 59 | 2024059 | 1E-100 >gi\|3176874 (AF065639) |
| | | cucumisin-like serine protease |
| | | [Arabidopsis thaliana] Length = 757 |
| 60 | 2024060 | 1E-39 >gb\|AAD23390.1\|AF109733_1 |
| | | (AF109733) SWI/SNF-related, matrix-associated, |
| | | actin-dependent regulator of chromatin D1 |
| | | [Homo sapiens] Length = 453 |
| 61 | 2024061 | Pkc_Phospho_Site(108-110) |
| 62 | 2024062 | Tyr_Phospho_Site(223-230) |
| 63 | 2024063 | Tyr_Phospho_Site(712-719) |
| 64 | 2024064 | 7E-87 >gi\|3643604 (AC005395) |
| | | receptor-like protein kinase [Arabidopsis |
| | | thaliana] Length = 960 |
| 65 | 2024065 | 2E-24 >ref\|NP_006317.1\|PN\|FIE14\| seven transmem- |
| | | brane domain protein >gi\|3550427\|emb\|CAA77013\| |
| | | (Y18007) seven transmembrane domain protein |
| | | [Homo sapiens] Length = 224 |
| 66 | 2024066 | Tyr_Phospho_Site(124-130) |
| 67 | 2024067 | 1E-106 ) >gi\|2353175 (AF015544) |
| | | sigma factor 3 Arabidopsis thali- |
| | | ana] >gi\|2463554\|dbj\|BAA22530\| (D89994) SigC |
| | | [Arabidopsis thaliana] >gi\|5478585\|dbj\|BAA82450.1\| |
| | | (AB019944) sigma factor SigC [Arabidopsis |
| | | thaliana] Length = 571 |
| 68 | 2024068 | Tyr_Phospho_Site(760-766) |
| 69 | 2024069 | 1E-49 >emb\|CAB45546.1\| (AJ007014) |
| | | AMMECR1 protein [Homo |
| | | sapiens] >gi\|6063688\|emb\|CAB58122.1\| |
| | | (AJ012221) AMMECRi [Homo sapiens] Length = 333 |
| 70 | 2024070 | 1E-111 >emb\|CAA16556\| (AL021635) |
| | | cytochrome P450 - like protein [Arabidopsis thaliana] |
| | | Length = 526 |
| 71 | 2024071 | 2E-69 >gb\|AAD40132.1\|AF149413_13 (AF149413) |
| | | contains similarity to arabinosidase [Arabidopsis |
| | | thaliana] Length = 521 |
| 72 | 2024072 | 5E-35 >gi\|556409 (L34551) transcriptional |
| | | activator protein [Oryza sativa] Length = 298 |
| 73 | 2024073 | Tyr_Phospho_Site(81 8-825) |
| 74 | 2024074 | SE-13 >dbj\|BAA84689.1\| (AB025218) |
| | | Sid394p [Mus musculus] Length = 201 |
| 75 | 2024075 | 3E-94 >gb\|AAD55640.1\|AC008017_13 (AC008017) |
| | | Similar to downy mildew resistance protein RPP5 |
| | | [Arabidopsis thaliana] Length = 176 |
| 76 | 2024076 | 9E-82 ) >emb\|CAA18722.1\| (AL022603) |
| | | NADPH quinone oxidoreductase [Arabidopsis |
| | | thaliana] >gi\|4455266\|emb\|CAB36802.1\| |
| | | (AL035527) NADPH guinone oxidoreductase |
| | | [Arabidopsis thaliana] Length = 325 |
| 77 | 2024077 | 4E-87 >emb\|CAA10060.1\| (AJ012571) |
| | | glutathione transferase [Arabidopsis |
| | | thaliana] Length = 219 |
| 78 | 2024078 | Rnp_1(214-221) |
| 79 | 2024079 | 7E-19 >gb\|AAD23884.1\|AC006954_5 (AC006954) |
| | | glucosyltransferase [Arabidopsis thaliana] |
| | | Length = 690 |
| 80 | 2024080 | Tyr_Phospho_Site(1175-1182) |
| 81 | 2024081 | Rgd(310-312) |
| 82 | 2024082 | 8E-79 >emb\|CAA72093\| (Y11210) |
| | | uracil phosphoribosyltransferase |
| | | [Nicotiana tabacum] Length = 224 |
| 83 | 2024083 | 3E-23 >emb\|CAB10416.1\| (Z97341) salt-inducible |
| | | protein homolog [Arabidopsis thaliana] |
| | | Length = 777 |
| 84 | 2024084 | Tyr_Phospho_Site(819-825) |
| 85 | 2024085 | 1E-126 >gb\|AAB53101.2\| (U68219) |
| | | catalase [Brassica napus] Length = 492 |
| 86 | 2024086 | Tyr_Phospho_Site(35-43) |
| 87 | 2024087 | 1E-100 >sp\|O23627\|SYG_ARATH GLYCYL- |
| | | TRNA SYNTHETASE (GLYCINE—TRNA |
| | | LIGASE) (GLYRS) >gi\|2564215\|emb\|CAA051621\| |
| | | (AJ002062) glycyl-tRNA synthetase [Arabidopsis |
| | | thaliana] Length = 729 |
| 88 | 2024088 | Pkc_Phospho_Site(122-124) |
| 89 | 2024089 | 2E-11 >gi\|3004821 (AF024691) |
| | | inorganic phosphate cotransporter |
| | | [Drosophila ananassae] Length = 483 |
| 90 | 2024090 | 5E-15 >gi\|707021 (U78721) |
| | | Ubiquitin-conjugating enzyme, E2-16kD isolog |
| | | [Arabidopsis thaliana] Length = 182 |
| 91 | 2024091 | 1E-113 >gi\|2384675 (AF012659) |
| | | potassium transporter AtKT4p |
| | | [Arabidopsis thaliana] Length = 273 |
| 92 | 2024092 | 4E-67 >gb\|AAD20169\| (AC006418) |
| | | zinc finger protein [Arabidopsis |
| | | thaliana] Length = 356 |
| 93 | 2024093 | 2E-89 >emb\|CAA65504∩ (X96728) |
| | | isocitrate dehydrogenase (NADP+) |
| | | [Nicotiana tabacum] Length = 470 |
| 94 | 2024094 | Tyr_Phospho_Site(2-10) |
| 95 | 2024095 | 5E-88 >emb\|CAA09728.1\| (AJ011669) |
| | | MYB96 protein [Arabidopsis thaliana] |
| | | Length = 343 |
| 96 | 2024096 | Pkc_Phospho_Site(38-40) |
| 97 | 2024097 | 2E-79 ) >gi\|2289010 (AC002335) |
| | | FKBP type peptidyl-prolyl cis-trans isomerase |
| | | isolog [Arabidopsis thaliana] Length = 167 |
| 98 | 2024098 | Pkc_Phospho_Site(79-81) |
| 99 | 2024099 | 3E-89 >gi\|1871194 (U90439) DNA |
| | | binding protein isolog [Arabidopsis |
| | | thaliana] >gi\|2335092 (AC002339) DNA binding |
| | | protein [Arabidopsis thaliana] |
| | | Length = 274 |
| 100 | 2024100 | 2E-49 >emb\|CAA07566\| (AJ007578) |
| | | pRIBS protein [Ribes nigrum] Length = 258 |
| 101 | 2024101 | 1E-64 >sp\|P15458\|2SS2_ARATH 2S SEED |
| | | STORAGE PROTEIN 2 PRECURSOR (2S |
| | | ALBUMIN STORAGE |
| | | PROTEIN) >gi\|68854\|pir\|\|NWMU2 2S albumin 2 |
| | | precursor - Arabidopsis thaliana >gi\|166615 (M22033) |
| | | albumin 2S subunit 2 precursor [Arabidopsis |
| | | thaliana] >gi\|395205\|emb\|CAA80871\| (Z24745) |
| | | 2S albumin isoform 2 [Arabidopsis |
| | | thaliana] >gi\|4490711\|emb\|CAB38845.1\| |
| | | (AL035680) NWMU2-25 albumin 2 precursor |
| | | [Arabidopsis thaliana] Length = 170 |
| 102 | 2024102 | 2E-26 >gb\|AAB01563.1\| (L47115) late |
| | | embryogenesis abundant protein |
| | | [Picea glauca] Length = 197 |
| 103 | 2024103 | Receptor_Cytokines_1(1172-1184) |
| 104 | 2024104 | 1E-106 ) >emb\|CAB41139.1\| (AL049658) |
| | | aldehyde dehydrogenase (NAD+)-like protein |
| | | [Arabidopsis thaliana] Length = 538 |
| 105 | 2024105 | 1E-144 ) >gb\|AAD25783.1\|AC00657_19 (AC006577) |
| | | Strong similarity to gb\|S77096 aldehyde |
| | | dehydrogenase homolog from Brassica napus and is a |
| | | member of PF\|00171 Aldehyde dehydrogenase family. |
| | | ESTs gb\|T46213, gb\|T42164, gb\|T43682, |
| | | gb\|N96380, gb\|T42973, gb... Length = 508 |
| 106 | 2024106 | 3E-21 >dbj\|BAA19751\| (D85339) |
| | | hydroxypyruvate reductase [Arabidopsis |
| | | thaliana] Length = 386 |
| 107 | 2024107 | Tyr_Phospho_Site(1123-1131) |
| 108 | 2024108 | Pkc_Phospho_Site(223-225) |
| 109 | 2024109 | 4E-86 >sp\|P25864\|RK9_ARATH 50S |
| | | RIBOSOMAL PROTEIN L9, CHLOROPLAST |
| | | PRECURSOR (CL9) >gi\|7l257\|pir\|\|R5MUL9 |
| | | ribosomal protein L9 precursor, chloroplast - |
| | | Arabidopsis thaliana >gi\|16499\|emb\|CAA77480\| |
| | | (Z11129) plastid ribosomal protein CL9 [Arabidopsis |
| | | thaliana] >gi\|16501\|emb\|CAA77594\| (Z11509) |
| | | Chloroplast ribosomal protein CL9 |
| | | [Arabidopsis thaliana] Length = 197 |
| 110 | 2024110 | 2E-84 ) >gb\|AAD23027.1\|AC006585_22 (AC006585) |
| | | tyrosine transaminase |
| | | [Arabidopsis thaliana] Length = 445 |
| 111 | 2024111 | 7E-30 >emb\|CAA16677\| (AL021684) |
| | | LRR-like protein [Arabidopsis |
| | | thaliana] Length = 445 |
| 112 | 2024112 | Pkc_Phospho_Site(35-37) |
| 113 | 2024113 | Rgd(1356-1358) |
| 114 | 2024114 | Pkc_Phospho_Site(40-42) |
| 115 | 2024115 | 2E-75 >sp\|Q08112\|RS15_ARATH 40S |
| | | RIBOSOMAL PROTEIN S15 >gi\|629556\|pir\|\|S43412 |
| | | ribosomal protein S15 - Arabidopsis |
| | | thaliana >gi\|313152\|emb\|CAA8O679\| (Z23161) |
| | | ribosomal protein S15 [Arabidopsis |
| | | thaliana] >gi\|313188\|emb\|CAA8O681\| (Z23162) |
| | | ribosomal protein S15 [Arabidopsis |
| | | thaliana] >gi\|1903366\|gb\|AAB70449\| (AC000104) |
| | | Strong similarity to Oryza 40S ribosomal protein S15. |
| | | ESTs gb\|R29788,gb\|ATTS0365 come from this |
| | | gene. [Arabidopsis thaliana] Length = 152 |
| 116 | 2024116 | Tyr_Phospho_Site(728-735) |
| 117 | 2024117 | 1E-78 >sp″O23255\|SAHH_ARATH ADEN- |
| | | OSYLHOMOCYSTEINASE (S-ADENOSYL-L- |
| | | HOMOCYSTEINE HYDROLASE) |
| | | (ADOHCYASE) >gi\|2244750\|emb\|CAB10173.1\| |
| | | (Z97335) adenosylhomocysteinase [Arabidopsis |
| | | thaliana] >gi\|3088579\|gb\|AAC14714.1\| |
| | | (AF059581) S-adenosyl-L-homocysteine hydrolase |
| | | [Arabidopsis thaliana] Length = 485 |
| 118 | 2024118 | 3E-45 >emb\|CAB39S95.1\| (AL049480) |
| | | ribosomal protein S10 [Arabidopsis |
| | | thaliana] Length = 177 |
| 119 | 2024119 | Tyr_Phospho_Site(759-767) |
| 120 | 2024120 | 1E-99 >pir\|\|S71168 envelope |
| | | Ca2+-ATPase precursor - Arabidopsis |
| | | thaliana >gi\|471089\|dbj\|BAA03091\| (D13984) |
| | | chloroplast envelope Ca2+-ATPase precursor [Ara- |
| | | bidopsis thaliana] >gi\|4165448\|emb\|CAA49558\| |
| | | (X69940) envelope Ca2+-ATPase [Arabidopsis |
| | | thaliana] Length = 946 |
| 121 | 2024121 | 8E-11 >sp\|Q43111\|PME3_PHAVU PECTI- |
| | | NESTERASE 3 PRECURSOR (PECTIN METHYL- |
| | | ESTERASE 3) (PE 3) >gi\|1076515\|pir\|\|S53105 |
| | | pectinesterase precursor - kidney |
| | | bean >gi\|732913\|emb\|CAA59482\| (X85216) |
| | | pectinesterase [Phaseolus vulgaris] Length = 581 |
| 122 | 2024122 | 1E-31 >ref\|NP_005827.1\|PUK114\| translational |
| | | inhibitor protein |
| | | p14.5 >gi\|1717975\|sp\|P52758\|UK14_HUMAN 14.5 |
| | | KD TRANSLATIONAL INHIBITOR |
| | | PROTEIN (P14.5) (UK114 ANTIGEN |
| | | HOMOLOG) >gi\|1177435\|emb\|CAA64670\| |
| | | (X95384) 14.5 kDa translational inhibitor protein, |
| | | p14.5 [Homo sapiens] Length = 1 |
| 123 | 2024123 | 1E-100 ) >sp\|P41377\|IF42_ARATH |
| | | EUKARYOTIC INITIATION FACTOR 4A-2 |
| | | (EIF-4A-2) >gi\|322504\|pir\|\|JC1453 translation |
| | | initiation factor elF-4A2 - Arabidopsis |
| | | thaliana >gi\|16556\|emb\|CAA46189\| (X65053) |
| | | eukaryotic translation initiation f |
| 124 | 2024124 | Tyr_Phospho_Site(676-682) |
| 125 | 2024125 | 2E-53 >gb\|AAD51854.1\|AF178990_1 (AF178990) |
| | | stress related protein [Vitis |
| | | niparia] Length = 248 |
| 126 | 2024126 | 6E-73 ) >sp\|Q42418\|PRO2_ARATH PROFILIN |
| | | 2 >gi\|1353766 (U43323) profilin 2 [Arabidopsis |
| | | thaliana] >gi\|1353772 (U43326) profilin 2 |
| | | [Arabidopsis thaliana] Length = 131 |
| 127 | 2024127 | 9E-82 >pir\|1558500 auxin-induced protein IAA9 - |
| | | Arabidopsis thaliana Length = 338 |
| 128 | 2024128 | 1E-98 >dbj\|BAA77603.1\| (AB027002) |
| | | plastidic aldolase [Nicotiana |
| | | paniculata] Length = 398 |
| 129 | 2024129 | 1E-19 >ref\|NP_006275.1\|PTAF2H\| TATA |
| | | box binding protein (TBP)-associated factor, RNA |
| | | polymerase II, H, |
| | | 30kD >gi\|3024688\|sp\|Q12962\|T2D8 HUMAN |
| | | TRANSCRIPTION INITIATION FACTOR |
| | | TFIID 30 KD SUBUNIT (TAFII-30) |
| | | (TAFII30) >gi\|627645\|pir\| |
| 130 | 2024130 | Tyr_Phospho_Site(699-706) |
| 131 | 2024131 | Tyr_Phospho_Site(394-401) |
| 132 | 2024132 | 3E-88 >emb\|CAA74696.1\| (Y14316) |
| | | MAP3K gamma protein kinase |
| | | [Arabidopsis thaliana] Length = 372 |
| 133 | 2024133 | 1E-139 >sp\|P32962\|NRL2_ARATH NITRILASE |
| | | 2 >gi\|322548\|pir\|\|S31969 nitnilase (EC 3.5.5.1) - |
| | | Arabidopsis thaliana >gi\|22656\|emb\|CAA48377\| |
| | | (X68305) nitrilase II [Arabidopsis |
| | | thaliana] >gi\|508733 (U09958) nitrilase |
| | | [Arabidopsis thaliana] Length = 339 |
| 134 | 2024134 | Tyr_Phospho_Site(555-561) |
| 135 | 2024135 | 2E-55 >gb\|AAD21451.1\| (AC007017) |
| | | DNA-binding protein [Arabidopsis |
| | | thaliana] Length = 145 |
| 136 | 2024136 | 7E-26 >gi\|2191150 (AF007269) |
| | | similar to mitochondrial carrier family |
| | | [Arabidopsis thaliana] Length = 352 |
| 137 | 2024137 | Rgd(662-664) |
| 138 | 2024138 | 7E-73 >gi\|1755182 (U75202) |
| | | germin-like protein |
| | | [Arabidopsis thaliana] Length = 147 |
| 139 | 2024139 | 3E-14 >gi\|1707012 (U78721) |
| | | tyrosyl-tRNA synthetase isolog |
| | | [Arabidopsis thaliana] Length 460 |
| 140 | 2024140 | Tyr_Phospho_Site(1124-1132) |
| 141 | 2024141 | 1E-70 >gi\|2924784 (AC002334) |
| | | similar to jasmonate inducible protein |
| | | [Arabidopsis thaliana] Length = 471 |
| 142 | 2024142 | 1E-112 >sp\|O04663\|IFE2_ARATH EUKARYOTIC |
| | | TRANSLATION INITIATION FACTOR |
| | | 4E (EIF-4E) (EIF4E) (MRNA CAP-BINDING |
| | | PROTEIN) (EIF-(ISO)4F 25 KD SUBUNIT) |
| | | (EIF-(ISO)4F P28 SUBUNIT) >gi\|2209274 |
| | | (U62044) eukaryotic initiation factor (iso)- |
| 143 | 2024143 | 2E-82 >gi\|2801536 (AF039531) |
| | | lysophospholipase homolog [Oryza |
| | | sativa] Length = 304 |
| 144 | 2024144 | 6E-77 >emb\|CAB38210\| (AL035601) |
| | | cytochrome P450 monooxygenase (CYP91A2) |
| | | [Arabidopsis thaliana] Length = 500 |
| 145 | 2024145 | 7E-50 >emb\|CAA19765\| (AL031004) |
| | | RSZp22 splicing factor [Arabidopsis |
| | | thaliana] >gi\|3435094\|gb\|AAD12769.1\| |
| | | (AF033586) 9G8-like SR protein |
| | | [Arabidopsis thaliana] Length = 200 |
| 146 | 2024146 | 0 >gi\|4098647 (U80668) homogentisate |
| | | 1,2-dioxygenase [Arabidopsis thaliana] Length 461 |
| 147 | 2024147 | Tyr_Phospho_Site(967-974) |
| 148 | 2024148 | Tyr_Phospho_Site(950-956) |
| 149 | 2024149 | 9E-13 >emb\|CAB10420.1\| (Z97341) LET1 |
| | | like protein [Arabidopsis thaliana] |
| | | Length = 578 |
| 150 | 2024150 | 6E-96 >gi\|4220454 (AC006216) |
| | | Similar to gi\|341 3714 T19L18.21 |
| | | myrosinase-binding protein from Arabidopsis thaliana |
| | | BAC gb\|AC004747. ESTs gb\|65870 and gb\|T20812 |
| | | come from this gene. [Arabidopsis thaliana] |
| | | Length = 303 |
| 151 | 2024151 | 1E-100 >emb\|CAA74028.1\| (Y13694) |
| | | multicatalytic endopeptidase complex, |
| | | proteasome precursor, beta subunit [Arabidopsis |
| | | thaliana] >gi\|2827525\|emb\|CAAl 6533.1\| |
| | | (AL021633) multicatalytic endopeptidase complex, |
| | | proteasome precursor, beta subunit [Arabidopsis |
| | | thaliana] >gi\|3421099 (AF043529) 205 |
| | | proteasome subunit PBA1 [Arabidopsis |
| | | thaliana] Length = 233 |
| 152 | 2024152 | 3E-61 >emb\|CAB45873.1\| (Y19104) |
| | | beta-alanine synthase [Lycopersicon |
| | | esculentum] Length = 300 |
| 153 | 2024153 | 2E-11 >gi\|3482913 (AC003970) |
| | | Similar to MtN21, gi\|2598575, Megicago |
| | | truncatula nodulation induced gene [Arabidopsis |
| | | thaliana] Length = 385 |
| 154 | 2024154 | 9E-28 >sp\|Q46948\|THIJ_ECOLI 4-METHYL- |
| | | 5(B-HYDROXYETHYL)-THIAZOLE MONO- |
| | | PHOSPHATE BIOSYNTHESIS |
| | | ENZYME >gi\|1100872 (U34923) ThiJ [Escherichia |
| | | coli] >gi\|1773108 (U82664) 4-methyl-5(b- |
| | | hydroxyethyl)-thiazole monophosphate biosynthesis |
| | | protein [Escherichia coli] >gi\|1786626 |
| | | (AE000148) 4-methyl-5(beta-hydroxyethyl)-thiazole |
| | | monophosphate synthesis [Escherichia coli] |
| | | Length = 198 |
| 155 | 2024155 | Tyr_Phospho_Site(758-765) |
| 156 | 2024156 | Tyr_Phospho_Site(577-585) |
| 157 | 2024157 | 2E-84 >sp\|P2I218\|PCR_ARATH PROTO- |
| | | CHLOROPHYLLIDE REDUCTASE PRECURSOR |
| | | (PCR) (NADPH-PROTOCHLOROPHYLLIDE |
| | | OXIDOREDUCTASE) >gi\|968977 (U29785) |
| | | NADPH:protochlorophyllide |
| | | oxidoreductase B [Arabidopsis |
| | | thaliana] >gi\|4972069\|emb\|CAB43876.1\| |
| | | (AL078467) protochlorophyllide reductase |
| | | precursor [Arabidopsis |
| | | thaliana] >gi\|1583456\|prf\|\|2120441B |
| | | protochlorophyllide oxidoreductase [Arabidopsis |
| | | thaliana] Length = 401 |
| 158 | 2024158 | Rgd(1197-1199) |
| 159 | 2024159 | Tyr_Phospho_Site(24-32) |
| 160 | 2024160 | Tyr_Phospho_Site(642-649) |
| 161 | 2024161 | 1E-15 >gi\|2352492 (AF005047) |
| | | transport inhibitor response 1 [Arabidopsis |
| | | thaliana] >gi\|2352494 (AF005048) transport |
| | | inhibitor response 1 |
| | | [Arabidopsis thaliana] Length = 594 |
| 162 | 2024162 | Tyr_Phospho_Site(1187-1194) |
| 163 | 2024163 | Tyr_Phospho_Site(927-935) |
| 164 | 2024164 | 1E-115 >gb\|AAD15571\| (AC006340) |
| | | MADS-box protein AGLI7 |
| | | [Arabidopsis thaliana] Length = 227 |
| 165 | 2024165 | Tyr_Phospho_Site(723-729) |
| 166 | 2024166 | Tyr_Phospho_Site(841-848) |
| 167 | 2024167 | 3E-46 >sp\|P87068\|SYRP_LACBI SYMBIOSIS- |
| | | RELATED PROTEIN Length = 184 |
| 168 | 2024168 | 1E-103 >pir\|\|S66345 senescence-associated |
| | | protein sen1 - Arabidopsis thaliana >gi\|1046270 |
| | | (U26945) senescence-associated protein [Arabidopsis |
| | | thaliana] >gi\|3367595\|emb\|CAA20047\| (AL031135) |
| | | senescence-associated protein sen1 [Arabidopsis |
| | | thaliana] >gi\|3805843\|emb\|CAA21463.1\| (AL031986) |
| | | senescence-associated protein sen1 [Arabidopsis |
| | | thaliana] Length = 182 |
| 169 | 2024169 | 5E-36 >emb\|CAA09367\| (AJ010811) HB2 |
| | | homeodomain protein [Populus tremula x Populus |
| | | tremuloides] Length = 261 |
| 170 | 2024170 | Pkc_Phospho_Site(44-46) |
| 171 | 2024171 | 4E-33 >sp\|P17009\|RR10_CYAPA CYANELLE |
| | | 30S RIBOSOMAL PROTEIN |
| | | S10 >gi\|70927\|pir\|\|R3KT10 ribosomal protein |
| | | S10 - Cyanophora paradoxa |
| | | cyanelle >gi\|11391\|emb\|CAA36388\| (X52143) |
| | | ribosomal protein S10 (AA 1-105) |
| | | [Cyanophora paradoxa] |
| 172 | 2024172 | 8E-97 >sp\|P92959\|RK24_ARATH 50S |
| | | RIBOSOMAL PROTEIN L24, CHLOROPLAST |
| | | PRECURSOR >gi\|1694974\|emb\|CAA70851\| (Y09635) |
| | | plastid ribosomal protein [Arabidopsis thaliana] |
| | | Length = 198 |
| 173 | 2024173 | 1E-18 >pir\|S51352 probable membrane |
| | | protein YLR350w-yeast (Saccharomyces |
| | | cerevisiae) >gi\|609381 (U19028) Ylr350wp |
| | | [Saccharomyces cerevisiae] Length = 216 |
| 174 | 2024174 | 1E-125 >sp\|O04928\|CDS1_ARATH PHOS- |
| | | PHATIDATE CYTIDYLYLTRANSFERASE |
| | | (CDP-DIGLYCERIDE SYNTHETASE) (CDP- |
| | | DIGLYCERIDE PYROPHOSPHORYLASE) |
| | | (CDP-DIACYLGLYCEROL SYNTHASE) (CDS) |
| | | (CTP:PHOSPHATIDATE CYTIDYLYLTRANS- |
| | | FERASE) (CDP-DAG |
| | | SYNTHASE) >gi\|2181182\|emb\|CAA63969\| |
| | | (X94306) CDP-diacylglycerol synthetase |
| | | [Arabidopsis thaliana] Length = 421 |
| 175 | 2024175 | Tyr_Phospho_Site(527-533) |
| 176 | 2024176 | 8E-40 >gb\|AAD31347.1\|AC007212_3 (AC007212) |
| | | mitochondrial protein [Arabidopsis |
| | | thaliana] Length = 996 |
| 177 | 2024177 | Tyr_Phospho_Site(921-928) |
| 178 | 2024178 | 2E-20 >pir\|\|S67312 probable membrane |
| | | protein YDR255c - yeast (Saccharomyces |
| | | cerevisiae) >gi\|1136210\|emb\|CAA92712\| |
| | | (Z68329) unknown [Saccharomyces |
| | | cerevisiae] >gi\|1226031\|emb\|CAA94094\| |
| | | (Z70202) unknown [Saccharomyces |
| | | cerevisiae] Length = 421 |
| 179 | 2024179 | 2E-59 ) >gb\|AAD25609.1\|AC005287_11 |
| | | (AC005287) translation initiation factor |
| | | [Arabidopsis thaliana] Length = 113 |
| 180 | 2024180 | 2E-43 >emb\|CAB56692.1\| (AJ249794) |
| | | lipoxygenase [Arabidopsis thaliana] |
| | | Length = 919 |
| 181 | 2024181 | 1E-113 >emb\|CAB39679.1\| (AL049483) |
| | | beta-galactosidase [Arabidopsis |
| | | thaliana] Length = 729 |
| 182 | 2024182 | 3E-74 >emb\|CAA18498\| (AL022373) |
| | | DnaJ-Iike protein [Arabidopsis |
| | | thaliana] Length = 161 |
| 183 | 2024183 | Tyr_Phospho_Site(149-156) |
| 184 | 2024184 | Tyr_Phospho_Site(890-897) |
| 185 | 2024185 | Tyr_Phospho_Site(915-921) |
| 186 | 2024186 | 3E-12 >sp\|P72777\|Y54L_SYNY3 YCF54- |
| | | LIKE PROTEIN >gi\|1651865\|dbj\|BAA16792\| |
| | | (D90900) hypothetical protein [Synechocystis sp.] |
| | | Length = 133 |
| 187 | 2024187 | Tyr_Phospho_Site(1377-1384) |
| 188 | 2024188 | Tyr_Phospho_Site(163-171) |
| 189 | 2024189 | 1E-140 >gi\|2150027 (AF001269) |
| | | NADP-malic enzyme [Lycopersicon |
| | | esculentum] Length = 640 |
| 190 | 2024190 | Tyr_Phospho_Site(834-841) |
| 191 | 2024191 | IE-90 >sp\|P34790\|CYP1_ARATH PEPTIDYL- |
| | | PROLYL CIS-TRANS ISOMERASE (PPIASE) |
| | | (ROTAMASE) (CYCLOPHILIN) (CYCLOSPORIN |
| | | A-BINDING PROTEIN) >gi\|405129 |
| | | (L14844) cyclophilin [Arabidopsis |
| | | thaliana] >gi\|4490326\|emb\|CAB38608.1\| |
| | | (AL035656) peptidylprolyl isomerase ROC1 |
| | | [Arabidopsis thaliana] Length = 172 |
| 192 | 2024192 | 1E-31 >gi\|4115383 (AC005967) |
| | | receptor-like protein kinase [Arabidopsis |
| | | thaliana] Length = 809 |
| 193 | 2024193 | 3E-97 >pir\|\|S50141 peptidylprolyl |
| | | isomerase (EC 5.2.1.8) - Arabidopsis |
| | | thaliana >gi\|460968 (U07276) peptidyl-prolyl |
| | | cis-trans isomerase [Arabidopsis |
| | | thaliana] >gi\|992643 (U32186) cyclophilin |
| | | [Arabidopsis thaliana] >gi |
| 194 | 2024194 | Tyr_Phospho_Site(408-415) |
| 195 | 2024195 | Tyr_Phospho_Site(777-784) |
| 196 | 2024196 | Pkc_Phospho_Site(20-22) |
| 197 | 2024197 | Tyr_Phospho_Site(402-409) |
| 198 | 2024198 | 3E-25 >gi\|2688824 (U93273) |
| | | auxin-repressed protein [Prunus |
| | | armeniaca] Length = 133 |
| 199 | 2024199 | Tyr_Phospho_Site(31-38) |
| 200 | 2024200 | Pkc_Phospho_Site(28-30) |
| 201 | 2024201 | Pkc_Phospho_Site(25-27) |
| 202 | 2024202 | Pkc_Phospho_Site(43-45) |
| 203 | 2024203 | 1E-42 >gb\|AAD26876.1\|AC007230_10 |
| | | (AC007230) Belongs to PF100026 |
| | | Eukaryotic aspartyl protease family. |
| | | [Arabidopsis thaliana] Length = 449 |
| 204 | 2024204 | 1E-112 >gi\|1669387 (U41998) |
| | | actin 2 [Arabidopsis thaliana] Length = 377 |
| 205 | 2024205 | 1E-69 >sp\|P52410\|FABB_ARATH 3- |
| | | OXOACYL-[ACYL-CARRIER-PROTEIN] |
| | | SYNTHASE I PRECURSOR (BETA- |
| | | KETOACYL-ACP SYNTHASE I) |
| | | (KAS I) >gi\|780814 (U24177) 3-ketoacyl-acyl |
| | | carrier protein synthase I [Arabidopsis |
| | | thaliana] Length = 473 |
| 206 | 2024206 | 1E-111) >gi\|2281094 (AC002333) |
| | | molybdenum cofactor biosynthesis protein E isolog |
| | | [Arabidopsis thaliana] >gi\|4469121\|emb\|CAB38428\| |
| | | (AJ133519) molybdopterin synthase large subunit |
| | | [Arabidopsis thaliana] Length = 198 |
| 207 | 2024207 | 1E-45 >emb\|CAB41717.1\| (AL049730) |
| | | pEARLI 1-like protein [Arabidopsis |
| | | thaliana] Length = 161 |
| 208 | 2024208 | 2E-49 >gb\|AAD20164\| (AC006418) auxin |
| | | response factor 1 [Arabidopsis |
| | | thaliana] Length = 622 |
| 209 | 2024209 | Tyr_Phospho_Site(292-299) |
| 210 | 2024210 | 3E-97 >pir\|\|S52771 beta-glucosidase |
| | | (EC 3.2.1.21) - rape >gi\|757740\|emb\|CAA57913\| |
| | | (X82577) beta-glucosidase [Brassica napus] |
| | | Length = 514 |
| 211 | 2024211 | 5E-85 >gb\|AAD24852.1\|AC007071_24 |
| | | (AC007071) 40S ribosomal protein; |
| | | contains C-terminal domain [Arabidopsis thaliana] |
| | | Length = 250 |
| 212 | 2024212 | 7E-95 >dbj\|BAA3l585.1\| (AB016066) |
| | | mitochondrial phosphate transporter |
| | | [Arabidopsis thaliana] Length = 288 |
| 213 | 2024213 | 4E-17 >gi\|4102690 (AF004806) 24 |
| | | kDa seed maturation protein [Glycine |
| | | maxi Length = 212 |
| 214 | 2024214 | Tyr_Phospho_Site(40-47) |
| 215 | 2024215 | 3E-85 >gb\|AAD20423\| (AC007019) |
| | | RAS-related protein RAB7 |
| | | [Arabidopsis thaliana] Length = 230 |
| 216 | 2024216 | Tyr_Phospho_Site(1316-1323) |
| 217 | 2024217 | 1E-151 >gi\|2281088 (AC002333) |
| | | indole-3-acetate beta-glucosyltransferase |
| | | isolog [Arabidopsis thaliana] Length = 449 |
| 218 | 2024218 | 1E-160 >sp\|P37702\|MYRO_ARATH MYRO- |
| | | SINASE PRECURSOR (SINIGRINASE) |
| | | (THIOGLUCOSIDASE) >gi\|1362006\|pir\|\|S56653 |
| | | thioglucosidase (EC 3.2.3.1) - Arabidopsis |
| | | thaliana >gi\|304115 (L11454) thioglucosidase |
| | | [Arabidopsis thaliana] >gi\|871990 |
| 219 | 2024219 | Zinc_Finger_C2h2(1007-1028) |
| 220 | 2024220 | 2E-39 >sp\|Q03251\|GRP8_ARATH GLYCINE- |
| | | RICH RNA-BINDING PROTEIN 8 (CCRI |
| | | PROTEIN) >gi\|419756\|pir\|\|S30148 |
| | | glycine-rich protein (clone AtGRP8) - Arabidopsis |
| | | thaliana >gi\|16305\|emb\|CAA78712\|(Z14988) |
| | | glycine rich protein [Arabidopsis |
| | | thaliana] >gi\|166658 (L04171) ORE |
| | | [Arabidopsis thaliana] >gi\|166839 |
| | | (L00649) RNA-binding protein [Arabidopsis |
| | | thaliana] >gi\|4914438\|emb\|CAB43641.1\| |
| | | (AL050351) glycine-rich protein (clone AtGRP8) |
| | | [Arabidopsis thaliana] Length 169 |
| 221 | 2024221 | 2E-52 >sp\|O22060\|SPS1_CITUN SUCROSE- |
| | | PHOSPHATE SYNTHASE 1 (UDP-GLUCOSE- |
| | | FRUCTOSE-PHOSPHATE GLUCOSYLTRANS- |
| | | FERASE 1) >gi\|2588888\|dbj\|BAA23213\| |
| | | (AB005023) sucrose-phosphate synthase [Citrus |
| | | unshiu] Length = 1057 |
| 222 | 2024222 | 2E-84 >emb\|CAA17163\| (AL021890) |
| | | peroxidase prxrl [Arabidopsis |
| | | thaliana] >gi\|2961341\|emb\|CAA18099.1\| |
| | | (AL022140) peroxidase prxr1 |
| | | [Arabidopsis thaliana] Length = 323 |
| 223 | 2024223 | Pkc_Phospho_Site(28-30) |
| 224 | 2024224 | Tyr_Phospho_Site(100-107) |
| 225 | 2024225 | 8E-42 >gi\|3600033 (AF080119) contains |
| | | similarity to the N terminal domain of the E1 protein |
| | | (Pfam: E1_N.hmm, score: 12.36) [Arabidopsis |
| | | thaliana] Length = 1074 |
| 226 | 2024226 | 2E-85 >sp\|P46645\|AAT2_ARATH ASPARTATE |
| | | AMINOTRANSFERASE, CYTOPLASMIC |
| | | ISOZYME 1 (TRANSAMINASE |
| | | A) >gi\|693690 (U15033) aspartate aminotransferase |
| | | [Arabidopsis thaliana] Length = 405 |
| 227 | 2024227 | Tyr_Phospho_Site(737-744) |
| 228 | 2024228 | Tyr_Phospho_Site(819-827) |
| 229 | 2024229 | Tyr_Phospho_Site(707-714) |
| 230 | 2024230 | 7E-37 >sp\|P02308\|H4_WHEAT HISTONE |
| | | H4 >gi\|70771\|pir\|\|HSZM4 histone H4 - |
| | | maize >gi\|81642\|pir\|\|S06904 histone |
| | | H4 -Arabidopsis thaliana >gi\|2119028\|pir\|\|S60475 |
| | | histone H4 - garden pea >gi\|21795\|emb\|CAA24924\| |
| | | (X00043) histone H4 [Triticum aestivum] >gi\|166740 |
| | | (M17132) histone H4 [Arabidopsis |
| | | thaliana] >gi\|166742 (M17133) histone H4 |
| | | [Arabidopsis thaliana] >gi\|168499 (M36659) |
| | | histone H4 (H4C13) [Zea mays] >gi\|168501 |
| | | (M13370) histone H4 [Zea mays] >gi\|168503 |
| | | (M13377) histone H4 [Zea mays] >gi\|498898 |
| | | (U10042) histone H4 homolog [Pisum |
| | | sativum] >gi\|1806285\|emb\|CAB01914\| |
| | | (Z79638) histone H4 homologue [Sesbania |
| | | rostrata] >gi\|3927823 (AC005727) |
| | | histone H4 [Arabidopsis |
| | | thaliana] >gi\|4580385\|gb\|AAD24364.1\|AC007184_4 |
| | | (AC007184) histone H4 [Arabidopsis |
| | | thaliana] >gi\|6009915\|dbj\|BAA85120.1\| |
| | | (AB018245) histone H4-like protein [Solanum |
| | | melongena] >gi\|225838\|prf\|\|1314298A histone |
| | | H4 [Arabidopsis thaliana] Length = 103 |
| 231 | 2024231 | 2E-60 >sp\|P39925\|AFG3_YEAST MITO- |
| | | CHONDRIAL RESPIRATORY CHAIN |
| | | COMPLEXES ASSEMBLY PROTEIN AFG3 |
| | | (TAT-BINDING HOMOLOG |
| | | 10) >gi\|626985\|pir\|\|S46611 YTA10 protein - yeast |
| | | (Sacoharomyces cerevisiae) >gi\|531750\|emb\|CAA56953\| |
| | | (X81066) probable mitochondrial protein |
| | | [Saccharomyces cerevisiae] >gi\|603609 (U18778) |
| | | Afg3p [Saccharomyces cerevisiae] Length = 761 |
| 232 | 2024232 | 8E-60 >gi\|3850579 (AC005278) Strong |
| | | similarity to gb\|D14550 extracellular dermal |
| | | glycoprotein (EDGP) precursor from Daucus carota. |
| | | ESTs gb\|H37281, gb\|T44167, gb\|T21813, gb\|N38437, |
| | | gb\|Z26470, gb\|R65072, gb\|N76373, gb\|F15470, |
| | | gb\|Z35182, gb\|H76373, gb\|Z34678 an... |
| | | Length = 433 |
| 233 | 2024233 | Tyr_Phospho_Site(1019-1026) |
| 234 | 2024234 | 3E-13 >gb\|AAD28548.1\|AF111941_1 (AF111941) |
| | | development protein DG1148 [Dictyostelium |
| | | discoideum] Length = 306 |
| 235 | 2024235 | 1E-62 >gi\|1256595 (U38915) LAB |
| | | [Synechocystis PCC6803] Length = 379 |
| 236 | 2024236 | Pkc_Phospho_Site(4-6) |
| 237 | 2024237 | Tyr_Phospho_Site(742-749) |
| 238 | 2024238 | Pkc_Phospho_Site(7-9) |
| 239 | 2024239 | 1E-100 >gb\|AAD14483\| (AC005966) |
| | | Strong similarity to gb\|AF061286 gamma-adaptin 1 |
| | | from Arabidopsis thaliana. EST gb\|H37393 comes |
| | | from this gene. [Arabidopsis thaliana] |
| | | Length = 867 |
| 240 | 2024240 | 3E-84 >sp\|P41127\|RL13_ARATH 60S |
| | | RIBOSOMAL PROTEIN L13 (BBC1 |
| | | PROTEIN HOMOLOG) >gi\|480787\|pir\|\|S37271 |
| | | ribosomal protein L13 - Arabidopsis |
| | | thaliana >gi\|404166\|emb\|CAA53005\| (X75162) |
| | | BBC1 protein [Arabidopsis thaliana] Length = 20 |
| 241 | 2024241 | 1E-105 >emb\|CAA06834.1\| (AJ006053) |
| | | peroxisomal membrane protein [Arabidopsis |
| | | thaliana] >gi\|4773886\|gb\|AAD29759.1\|AF076243_6 |
| | | (AF076243) pmp22 peroxisomal membrane protein |
| | | [Arabidopsis thaliana] Length = 190 |
| 242 | 2024242 | Rgd(1013-1015) |
| 243 | 2024243 | 3E-37 >gb\|AAD29842.1\|AF064694_1 (AF064694) |
| | | catechol O-methyltransferase; Omt II;THATU;2 |
| | | [Thalictrum tuberosum] Length = 362 |
| 244 | 2024244 | Rgd(653-655) |
| 245 | 2024245 | Tyr_Phospho_Site(632-638) |
| 246 | 2024246 | 1E-38 >gi\|1388088 (U35831) thioredoxin m |
| | | [Pisum sativum] Length = 172 |
| 247 | 2024247 | Tyr_Phospho_Site(1204-1211) |
| 248 | 2024248 | Tyr_Phospho_Site(314-320) |
| 249 | 2024249 | Pkc_Phospho_Site(71-73) |
| 250 | 2024250 | Tyr_Phospho_Site(963-970) |
| 251 | 2024251 | 4E-67 >gi\|1408471 (U48938) actin |
| | | depolymerizing factor 1 [Arabidopsis |
| | | thaliana] >gi\|3851707 (AF102173) actin |
| | | depolymerizing factor 1 [Arabidopsis |
| | | thaliana] Length = 139 |
| 252 | 2024252 | 9E-18 >gi\|2062157 (AC001645) jasmonate |
| | | inducible protein isolog [Arabidopsis thaliana] |
| | | Length = 705 |
| 253 | 2024253 | 2E-84 >gi\|3478700 (AF034387) AFT protein |
| | | [Arabidopsis thaliana] Length = 368 |
| 254 | 2024254 | 1E-128 ) >gi\|3169569 (AF062589) 3-keto- |
| | | acyl-CoA thiolase 2 [Arabidopsis |
| | | thaliana] >gi\|3220237 (AF062591) peroxisomal |
| | | 3-keto-acyl-CoA thiolase 2 precursor [Arabidopsis |
| | | thaliana] Length = 457 |
| 255 | 2024255 | 1E-83 >sp\|P35132\|UBC9_ARATH UBIQUITIN- |
| | | CONJUGATING ENZYME E2-17 KD 9 |
| | | (UBIQUITIN-PROTEIN LIGASE 9) |
| | | (UBIQUITIN CARRIER PROTEIN 9) |
| | | (UBCAT4B) >gi\|421 857\|pir\|\|S32674 ubiquitin-protein |
| | | ligase (EC 6.3.2.19) UBC9 - Arabidopsis |
| | | thaliana >gi\|297884\|emb\|CAA78714\| (Z14990) |
| | | ubiquitin conjugating enzyme homolog [Arabidopsis |
| | | thaliana] >gi\|349211 (L00639) ubiguitin |
| | | conjugating enzyme [Arabidopsis |
| | | thaliana] >gi1600391\|emb\|CAA51201\| |
| | | (X72626) ubiquitin conjugating enzyme E2 [Arabi- |
| | | dopsis thaliana] >gi\|4455355\|emb\|CAB36765.1\| |
| | | (AL035524) ubiguitin-protein ligase UBC9 [Arabidopsis |
| | | thaliana] Length = 148 |
| 256 | 2024256 | 3E-95 >gi\|3044218 (AF057144) signal |
| | | peptidase [Arabidopsis thaliana] |
| | | Length = 167 |
| 257 | 2024257 | 8E-75 >sp\|P34788\|RS18_ARATH 40S |
| | | RIBOSOMAL PROTEIN S18 >gi\|480908\|pir\|\|S37496 |
| | | ribosomal protein S18.A - Arabidopsis |
| | | thaliana >gi\|405613\|emb\|CAA80684\| (Z23165) |
| | | ribosomal protein S18A [Arabidopsis |
| | | thaliana] >gi\|434343\|emb\|CAA82273\| (Z28701) |
| | | S18 ribosomal protein [Arabidopsis |
| | | thaliana] >gi\|434345\|emb\|CAA82274\| (Z28702) |
| | | S18 ribosomal protein [Arabidopsis |
| | | thaliana] >gi\|434906\|emb\|CAA82275\| (Z28962) |
| | | S18 ribosomal protein [Arabidopsis |
| | | thaliana] >gi\|2505871\|emb\|CAA72909\| (Y12227) |
| | | ribosomal protein Si 8A [Arabidopsis |
| | | thaliana] >gi\|3287678 (AC003979) Match to |
| | | ribosomal S18 gene mRNA gb\|Z28701, DNA |
| | | gb\|Z23165 from A. thaliana. ESTs gb\|T21121, |
| | | gb\|Z17755, gb\|R64776 and gb\|R30430 come from |
| | | this gene. [Arabidopsis |
| | | thaliana] >gi\|4538910\|emb\|CAB39647.1\| |
| | | (AL049482) S18.A ribosomal protein [Arabidopsis |
| | | thaliana] Length = 152 |
| 258 | 2024258 | 2E-64 >gi\|3402679 (AC004697) unknown |
| | | protein [Arabidopsis thaliana] Length = 1029 |
| 259 | 2024259 | 2E-15 >emb\|CAA03859\| (AJ000016) Cks1 |
| | | protein [Arabidopsis |
| | | thaliana] >gi\|4510420\|gb\|AAD21506.1\| |
| | | (AC006929) cyclin-dependent kinase regulatory |
| | | subunit [Arabidopsis thaliana] Length = 87 |
| 260 | 2024260 | 1E-93 ) >dbj\|BAA82069.1\| (AB022330) |
| | | nClpP5 [Arabidopsis thaliana] Length = 387 |
| 261 | 2024261 | Pkc_Phospho_Site(22-24) |
| 262 | 2024262 | 1E-108 >sp\|O23515\|RL15_ARATH 60S RIBOSO- |
| | | MAL PROTEIN L15 >gi\|2245027\|emb\|CAB10447.1\| |
| | | (Z97341) ribosomal protein [Arabidopsis thaliana] |
| | | Length = 204 |
| 263 | 2024263 | 3E-86 >gi\|3953473 (AC002328) F2202.18 |
| | | [Arabidopsis thaliana] >gi\|5734520\|emb\|CAB52748.1\| |
| | | (AJ245630) photosystem I subunit V precursor |
| | | [Arabidopsis thaliana] Length = 160 |
| 264 | 2024264 | Pkc_Phospho_Site(66-68) |
| 265 | 2024265 | 1E-110 >gi\|3834324 (AC005679) Similar to |
| | | gb\|X92762 tafazzins protein from Homo sapiens. |
| | | [Arabidopsis thaliana] Length = 284 |
| 266 | 2024266 | 2E-90 >gi\|2511715 (AF019380) |
| | | phosphatidylinositol-4-phosphate 5-kinase |
| | | [Arabidopsis thaliana] Length = 752 |
| 267 | 2024267 | 5E-98 >sp\|O50061\|RK4_ARATH 50S RIBOSO- |
| | | MAL PROTEIN L4, CHLOROPLAST |
| | | PRECURSOR >gi\|2791998\|emb\|CAA74895\| |
| | | (Y14566) ribosomal protein L4 [Arabidopsis |
| | | thaliana] >gi\|2792000\|emb\|CAA74894\| |
| | | (Y14565) ribosomal protein L4 [Arabidopsis th |
| 268 | 2024268 | 1E-112 >gi\|2281631 (AF003096) AP2 |
| | | domain containing protein RAP2.3 |
| | | [Arabidopsis thaliana] Length = 248 |
| 269 | 2024269 | 2E-74 >emb\|CAA74175\| (Y13860) enoyl-ACP |
| | | reductase [Arabidopsis thaliana] Length 390 |
| 270 | 2024270 | 4E-82 >gi\|2352828 (AF009228) NaCl- |
| | | inducible Ca2+-binding protein |
| | | [Arabidopsis thaliana] Length = 155 |
| 271 | 2024271 | Tyr_Phospho_Site(115-122) |
| 272 | 2024272 | Pkc_Phospho_Site(35-37) |
| 273 | 2024273 | Tyr_Phospho_Site(1400-1407) |
| 274 | 2024274 | 1E-62 >gi\|3834310 (AC005679) Similar |
| | | to Ubiquitin-conjugating enzyme E2-17 KD gb\|D83004 |
| | | from Homo sapiens. ESTs gb\|T88233, gb\|Z24464, |
| | | gb\|N37265, gb\|H36151, gb\|Z34711, |
| | | gb\|AA040983, and gb\|T22122 come from this |
| | | gene. [Arabidopsis thaliana] Length 163 |
| 275 | 202427S | Pkc_Phospho_Site(35-37) |
| 276 | 2024276 | Pkc_Phospho_Site(41-43) |
| 277 | 2024277 | 1E-72 ) >g\|2316016 (U92650) MRP-like |
| | | ABC transporter [Arabidopsis |
| | | thaliana] Length = 1515 |
| 278 | 2024278 | Tyr_Phospho_Site(1356-1364) |
| 279 | 2024279 | Tyr_Phospho_Site(823-830) |
| 280 | 2024280 | Tyr_Phospho_Site(280-288) |
| 281 | 2024281 | Tyr_Phospho_Site(564-571) |
| 282 | 2024282 | Rgd(1265-1267) |
| 283 | 2024283 | Pkc_Phospho_Site(27-29) |
| 284 | 2024284 | 4E-62 >sp\|P37225\|MAON_SOLTU MALATE |
| | | OXIDOREDUCTASE [NAD] 59 KD ISOFORM, |
| | | MITOCHONDRIAL PRECURSOR (MALIC |
| | | ENZYME) (ME) (NAD-DEPENDENT MALIC |
| | | ENZYME) (NAD-ME) >gi\|1076666\|pir\|\|A53318 |
| | | malate dehydrogenase (decarboxylating) (EC 1.1.1.39) |
| | | 59K chain precursor, mitochondrial - |
| | | potato >gi\|438131\|emb\|CAA80547\| (Z23002) |
| | | precursor of the 59kDa subunit of the mitochondrial |
| | | NAD+-dependent malic enzyme [Solanum |
| | | tuberosum] Length = 601 |
| 285 | 2024285 | 5E-81 >sp\|P25069\|CAL2_ARATH CALMODULIN- |
| | | 2/3/5 >gi\|99671\|pir\|\|522503 calmodulin - |
| | | Arabidopsis thaliana >gi\|1076437\|pir\|\|S53006 |
| | | calmodulin - leaf mustard >gi\|2146726\|pir\|\|S71513 |
| | | calmodulin - Arabidopsis thaliana >gi\|166651 |
| | | (M38380) calmodulin-2 [Arabidopsis |
| | | thaliana] >gi\|166653 (M73711) calmodulin-3 |
| | | [Arabidopsis thaliana] >gi\|474183\|emb\|CAA47690\| |
| | | (X67273) calmodulin [Arabidopsis |
| | | thaliana] >gi\|497992 (U10150) calmodulin |
| | | [Brassica napus] >gi\|899058 (M88307) calmodulin |
| | | [Brassica juncea] >gi\|1183005\|dbj\|BAA08283\| |
| | | (D45848) calmodulin [Arabidopsis |
| | | thaliana] >gi\|3402706 (AC004261) unknown protein |
| | | [Arabidopsis thaliana] >gi\|3885333 (AC005623) |
| | | calmodulin [Arabidopsis |
| | | thaliana] >gi\|228407\|prf\|\|1803520A calmodulin 2 |
| | | [Arabidopsis thaliana] Length = 149 |
| 286 | 2024286 | 1E-100 >sp\|O23264\|SBP_ARATH SELENIUM- |
| | | BINDING PROTEIN >gi\|2244759\|emb\|CAB10182.1\| |
| | | (Z97335) selenium-binding protein like |
| | | [Arabidopsis thaliana] Length = 490 |
| 287 | 2024287 | 3E-58 >gi\|3831443 (AC005819) auxin- |
| | | regulated protein [Arabidopsis |
| | | thaliana] Length = 121 |
| 288 | 2024288 | Tyr_Phospho_Site(1271-1278) |
| 289 | 2024289 | 5E-43 >gi\|4235430 (AF098458) latex- |
| | | abundant protein [Hevea brasiliensis] Length = 417 |
| 290 | 2024290 | 3E-12 >dbj\|BAA77516.1\| (AB026987) a |
| | | dynamin-like protein ADL3 |
| | | [Arabidopsis thaliana] Length = 836 |
| 291 | 2024291 | 3E-86 >gi\|3386614 (AC004665) transcription |
| | | factor SF3 [Arabidopsis thaliana] Length = 226 |
| 292 | 2024292 | 2E-94 >gi\|1644427 (U74610) glyoxalase II |
| | | [Arabidopsis thaliana] Length = 256 |
| 293 | 2024293 | Pkc_Phospho_Site(76-78) |
| 294 | 2024294 | 4E-72 >sp\|O50003\|RL12_PRUAR 60S |
| | | RIBOSOMAL PROTEIN L12 >gi\|2677830 (U93168) |
| | | ribosomal protein L12 [Prunus armeniaca] |
| | | Length = 166 |
| 295 | 2024295 | 2E-85 ) >sp\|P35131\|UBC8_ARATH UBIQUITIN- |
| | | CONJUGATING ENZYME E2-17 KD 8 |
| | | (UBIQUITIN-PROTEIN LIGASE 8) |
| | | (UBIQUITIN CARRIER PROTEIN 8) |
| | | (UBCAT4A) >gi\|398699\|emb\|CAA78713\| (Z14989) |
| | | ubiquitin conjugating enzyme homolog [Arabidopsis thal |
| 296 | 2024296 | 5E-76 ) >emb\|CAA19826.1\| (AL031018) gamma- |
| | | glutamylcysteine synthetase [Arabidopsis |
| | | thaliana] >gi\|4262277\|gb\|AAD14544\| |
| | | (AF068299) gamma-glutamylcysteine synthetase |
| | | [Arabidopsis thaliana] Length = 522 |
| 297 | 2024297 | 4E-91 >emb\|CAB10561.1\| (Z97344) SUPERMAN |
| | | like protein [Arabidopsis thaliana] Length = 180 |
| 298 | 2024298 | 5E-97 >gi\|3582341 (AC005496) flavonol |
| | | 3-O-glucosyltransferase [Arabidopsis thaliana] |
| | | Length = 474 |
| 299 | 2024299 | Tyr_Phospho_Site(1285-1292) |
| 300 | 2024300 | 7E-12 >gi\|1208874 (U50071) C. elegans |
| | | ankyrin-refated unc-44 (GB:U21734) [Caenorhabditis |
| | | elegans] >gi\|1814197 (U39847) AO66 ankyrin |
| | | [Caenorhabditis elegans] Length = 1867 |
| 301 | 2024301 | 1E-119 >gi\|1235680 (U48698) receptor |
| | | serine/threonine kinase PR5K [Arabidopsis |
| | | thaliana] >gi\|1589714\|prf\|\|2211427A |
| | | receptor protein kinase [Arabidopsis |
| | | thaliana] Length = 665 |
| 302 | 2024302 | 1E-52 >gi\|2979554 (AC003680) CDC4 |
| | | like protein [Arabidopsis thaliana] |
| | | Length = 2946 |
| 303 | 2024303 | Tyr_Phospho_Site(203-211) |
| 304 | 2024304 | 1E-15 >gi\|1931652 (U95973) |
| | | phosphatidylinositol-4-phosphate 5-kinase |
| | | isolog [Arabidopsis thaliana] Length = 859 |
| 305 | 2024305 | 1E-118 >gi\|968975 (U29699) |
| | | NADPH:protochlorophyllide |
| | | oxidoreductase A [Arabidopsis |
| | | thaliana]>gi\|1583455\|prf\|\|2120441 A |
| | | protochiorophyllide oxidoreductase [Arabidopsis |
| | | thaliana] Length = 405 |
| 306 | 2024306 | Tyr_Phospho_Site(109-117) |
| 307 | 2024307 | 5E-77 >gi\|2576363 (U39783) amino |
| | | acid transport protein [Arabidopsis |
| | | thaliana] Length = 432 |
| 308 | 2024308 | 1E-48 >gi\|170131 (M55322) ribosomal |
| | | protein 30S subunit [Spinacia |
| | | oleracea] Length = 302 |
| 309 | 2024309 | Tyr_Phospho_Site(539-547) |
| 310 | 2024310 | Rgd(366-368) |
| 311 | 2024311 | 9E-99 >gi\|2565436 (AF028842) DegP |
| | | protease precursor [Arabidopsis |
| | | thaliana] Length = 437 |
| 312 | 2024312 | 4E-37 >gi\|2621768 (AE000848) ribonuclease |
| | | PH [Methanobacterium |
| | | thermoautotrophicum] Length = 240 |
| 313 | 2024313 | 3E-22 >emb\|CAB45787.1\| (AL080252) inositol |
| | | 1,3,4-trisphosphate 5/6-kinase-like protein |
| | | [Arabidopsis thaliana] Length = 338 |
| 314 | 2024314 | 2E-26 >gb\|AAD18154\| (AC006260) receptor |
| | | protein kinase [Arabidopsis thaliana] Length 961 |
| 315 | 2024315 | 4E-77 >sp\|P49730\|R1R2_TOBAC RIBO- |
| | | NUCLEOSIDE-DIPHOSPHATE REDUCTASE |
| | | SMALL CHAIN (RIBONUCLEOTIDE |
| | | REDUCTASE) (R2 |
| | | SUBUNIT) >gi\|1044912\|emb\|CAA63194\| |
| | | (X92443) ribonucleotide reductase R2 [Nicotiana |
| | | tabacum] Length = 329 |
| 316 | 2024316 | 4E-88 >gb\|AAD49760.1\|AC007932_8 (AC007932) |
| | | Similar to gi\|4982048 ribosomal protein L18 from |
| | | Thermotoga maritima genome gb\|AE001798. ESTs |
| | | gb\|Z3561 3, gb\|T75951, gb\|T22182, gb\|T45962, |
| | | gb\|H76281 and gb\|AI100025 come from this gene. |
| | | [Arabidopsis thaliana] Length = 170 |
| 317 | 2024317 | 1E-108 >emb\|CAB43186.1\| (AJ133753) peptide |
| | | methionine sulfoxide reductase [Arabidopsis |
| | | thaliana] >gi\|4884035\|emb\|CAB43187.1\| |
| | | (AJ133754) peptide methionine sulfoxide reductase |
| | | [Arabidopsis thaliana] Length = 204 |
| 318 | 2024318 | Pkc_Phospho_Site(222-224) |
| 319 | 2024319 | 1E-105 >gi\|2286069 (U72155) beta- |
| | | glucosidase [Arabidopsis thaliana] Length = 528 |
| 320 | 2024320 | 2E-18 >sp\|P43349\|TCTP_SOLTU TRANS- |
| | | LATIONALLY CONTROLLED TUMOR |
| | | PROTEIN HOMOLOG (TCTP) |
| | | (P23) >gi\|1072463\|pir\|\|A38959 IgE- |
| | | dependent histamine-releasing factor homolog - potato |
| | | >gi\|587546\|emb\|CAA85519\| (Z37160) |
| | | P23 protein [Solanum tuberosum] Length = 168 |
| 321 | 2024321 | Tyr_Phospho_Site(632-638) |
| 322 | 2024322 | 2E-67 >gi\|3785981 (AC005560) major |
| | | latex protein [Arabidopsis thaliana] Length = 151 |
| 323 | 2024323 | 1E-59 >sp\|Q39039\|VATL_ARATH VACUOLAR |
| | | ATP SYNTHASE 16 KD PROTEOLIPID |
| | | SUBUNIT (V-ATPASE 16 |
| | | KD PROTEOLIPID |
| | | SUBUNIT) >gi\|2118221\|pir\|\|S60132 H+-trans- |
| | | porting ATPase (EC 3.6.1.35), vacuolar, 16K chain (clone |
| | | AVA-P2) - Arabidopsis thaliana >gi\|926937 (L44585) |
| | | vacuolar H+-pumping ATPase 16 kDa proteolipid |
| | | [Arabidopsis thaliana] Length = 165 |
| 324 | 2024324 | 3E-55 >gi\|3355475 (AC004218) ribosomal |
| | | protein L23a [Arabidopsis thaliana] Length = 154 |
| 325 | 2024325 | 1E-118 >gi\|2344900 (AC002388) EREBP |
| | | isolog [Arabidopsis thaliana] Length = 226 |
| 326 | 2024326 | 4E-60 >gi\|3128187 (AC004521) beta- |
| | | glucosidase [Arabidopsis thaliana] Length = 506 |
| 327 | 2024327 | 2E-92 >gb\|AAD31339.1\|AC007354_12 (AC007354) |
| | | Similar to gb\|X02844 lipase precursor from |
| | | Staphylococcus hyicus. ESTs gb\|AI239406 and gb\|T76725 |
| | | come from this gene. [Arabidopsis thaliana] |
| | | Length = 473 |
| 328 | 2024328 | 2E-91 >emb\|CAB10172.1\| (Z97335) hydroxy- |
| | | methyltransferase [Arabidopsis |
| | | thaliana] Length = 471 |
| 329 | 2024329 | Tyr_Phospho_Site(586-593) |
| 330 | 2024330 | Tyr_Phospho_Site(76-83) |
| 331 | 2024331 | 1E-64 >dbj\|BAA34728\| (AB008489) response |
| | | regulator 6 [Arabidopsis thaliana] Length = 186 |
| 332 | 2024332 | Rgd(866-868) |
| 333 | 2024333 | Tyr_Phospho_Site(1174-1181) |
| 334 | 2024334 | Tyr_Phospho_Site(729-736) |
| 335 | 2024335 | 3E-84 ) >sp\|P42732\|RR13_ARATH 30S |
| | | RIBOSOMAL PROTEIN S13, |
| | | CHLOROPLAST PRECURSOR |
| | | (CS13) >gi\|2119093\|pir\|\|559594 ribosomal |
| | | protein S13 precursor, chloroplast - Arabidopsis |
| | | thaliana >gi\|16767\|emb\|CAA79013\| |
| | | (Z17611) chloroplast 30S |
| 336 | 2024336 | 8E-68 >sp\|P36210\|R12A_ARATH 60S |
| | | RIBOSOMAL PROTEIN L12-A, |
| | | CHLOROPLAST PRECURSOR |
| | | (CL12-A) >gi\|541895\|pir\|\|A53394 ribosomal |
| | | protein L12.A, chloroplast - Arabidopsis |
| | | thaliana >gi\|468771 \|emb\|CAA48181\| |
| | | (X68046) ribosomal protein L12 [Arabidopsis |
| | | thaliana] Length = 191 |
| 337 | 2024337 | 1E-84 ) >gi\|1657621 (U72505) G6p |
| | | [Arabidopsis thaliana] >gi\|3068711 |
| | | (AF049236) acyl-coA dehydrogenase [Arabidopsis |
| | | thaliana] >gi\|5478795\|jdb\|BAA82478.1\| |
| | | (AB017643) Short-chain acyl Co Aoxidase |
| | | [Arabidopsis thaliana] Length = 436 |
| 338 | 2024338 | Tyr_Phospho_Site(4-12) |
| 339 | 2024339 | 1E-85 >sp\|QO5085\|CHL1_ARATH NI- |
| | | TRATE/CHLORATE |
| | | TRANSPORTER >gi\|1076359\|pir\|\|A45772 |
| | | nitrate-inducible nitrate transporter - Arabidopsis |
| | | thaliana >gi\|166668 (L10357) CHL1 [Arabidopsis |
| | | thaliana] >gi\|3157921 (AC002131) Identical |
| | | to nitrate/chlorate transporter cDNA gb\|L10357 from |
| | | A. thaliana. ESTs gb\|H37533 and gb\|R29790, |
| | | gb\|T46117, gb\|T46068, gb\|T75688, gb\|R29817, |
| | | gb\|R29862, gb\|Z34634 and gb\|Z34258 come |
| | | from this gene. [Arabidopsis thaliana] Length = 590 |
| 340 | 2024340 | Tyr_Phospho_Site(40-47) |
| 341 | 2024341 | 2E-16 >emb\|CAB43916.1\| (AL078470) glycine- |
| | | rich protein like [Arabidopsis thaliana] |
| | | Length = 158 |
| 342 | 2024342 | 1E-106 >gb\|AAD31061.1\|AC007357_10 (AC007357) |
| | | Identical to gb\|D78605 cytochrome P450 mono- |
| | | oxygenase from Arabidopsis thaliana and is a member |
| | | of the PF\|00067 Cytochrome P450 family. ESTs |
| | | gb\|Z18072, gbjZ35218 and gb\|T43466 come from t |
| 343 2024343 | 1E-114 >sp\|P20363\|TBA3_ARATH TUBULIN | |
| | | ALPHA-3/ALPHA-5 |
| | | CHAIN >gi\|99768\|pir\|\|A32712 tubulin alpha-5 |
| | | chain - Arabidopsis thaliana >gi\|166912 |
| | | (M17189) alpha-tubulin [Arabidopsis |
| | | thaliana] >gi\|166918 (M84698) alpha-5 |
| | | tubulin [Arabido |
| 344 | 2024344 | 2E-99 ) >sp\|P10796\|RBS2_ARATH RIBULOSE |
| | | BISPHOSPHATE CARBOXYLASE SMALL CHAIN |
| | | 1 B PRECURSOR (RUBISCO SMALL SUBUNIT |
| | | 1 B) >gi\|68062\|pir\|\|RKMUB1 ribulose-bisphosphate |
| | | carboxylase (EC 4.1.1.39) small chain Bi precursor - |
| | | Arabidopsis th |
| 345 | 2024345 | 2E-61 >dbj\|BAA33810.1\| (AB018441) phi-1 |
| | | [Nicotiana tabacum] Length = 313 |
| 346 | 2024346 | 2E-58 >gb\|AAD48837.1\|AF166351_1 (AF166351) |
| | | alanine:glyoxylate aminotransferase 2 homolog |
| | | [Arabidopsis thaliana] Length = 476 |
| 347 | 2024347 | Tyr_Phospho_Site(305-312) |
| 348 | 2024348 | Pkc_Phospho_Site(118-120) |
| 349 | 2024349 | Zinc_Finger C2h2(14-35) |
| 350 | 2024350 | Tyr_Phospho_Site(601-608) |
| 351 | 2024351 | 8E-40 >emb\|CAB36810.1\| (AL035527) |
| | | spliceosome associated protein-like |
| | | [Arabidopsis thaliana] Length = 700 |
| 352 | 2024352 | Pkc_Phospho_Site(65-67) |
| 353 | 2024353 | 5E-86 >emb\|CAA67334\| (X98802) peroxi- |
| | | dase ATP11 a [Arabidopsis thaliana] >gi\|2388572 |
| | | (AC000098) Strong similarity to Arabidopsis |
| | | peroxidase ATP11A (gb\|X98802). [Arabidopsis |
| | | thaliana] >gi\|2388573 (AC000098) Strong |
| | | similarity to Arabidopsis peroxidase ATP11A |
| | | (gb\|X98802). [Arabidopsis thaliana] |
| | | Length = 325 |
| 354 | 2024354 | Tyr_Phospho_Site(846-853) |
| 355 | 2024355 | 3E-52 ) >gb\|AAD57002.1\|AC009465_16 (AC009465) |
| | | zeta-carotene desaturase precursor [Arabidopsis |
| | | thaliana] Length = 558 |
| 356 | 2024356 | 1E-147 >sp\|P30302\|WC2C_ARATH PLASMA |
| | | MEMBRANE INTRINSIC PROTEIN 2C |
| | | (WATER-STRESS INDUCED TONOPLAST |
| | | INTRINSIC PROTEIN) |
| | | (WSI-TIP) >gi\|2l 7869\|dbj\|BAA02520\| (D13254) |
| | | transmembrane channel protein [Arabidopsis |
| | | thaliana] >gi\|4371283\|g |
| 357 | 2024357 | Pkc_Phospho_Site(107-109) |
| 358 | 2024358 | Pkc_Phospho_Site(78-80) |
| 359 | 2024359 | Tyr_Phospho_Site(1075-1083) |
| 360 | 2024360 | 4E-32 >gb\|AAD39286.1\|AC007576_9 (AC007576) |
| | | Similar to protein kinases [Arabidopsis |
| | | thaliana] Length = 438 |
| 361 | 2024361 | 1E-95 >emb\|CAA18833.1\| (AL023094) isoflavone |
| | | reductase-like protein [Arabidopsis thaliana] |
| | | Length = 306 |
| 362 | 2024362 | Tyr_Phospho_Site(433-441) |
| 363 | 2024363 | Tyr_Phospho_Site(156-163) |
| 364 | 2024364 | 3E-93 >sp\|P42796\|R112_ARATH 60S |
| | | RIBOSOMAL PROTEIN L11B |
| | | (L16B) >gi\|550547\|emb\|CAA57396\| (X81800) |
| | | ribosomal protein L16 [Arabidopsis |
| | | thaliana] >gi\|4539392\|emb\|CAB37458.1\| |
| | | (AL035526) ribosomal protein L11, cytosolic |
| | | [Arabidopsis thaliana] Length = 184 |
| 365 | 2024365 | Tyr_Phospho_Site(250-258) |
| 366 | 2024366 | 0 >emb\|CAB40990.1 \| (AL049640) pollen |
| | | surface protein [Arabidopsis thaliana] |
| | | Length = 403 |
| 367 | 2024367 | 6E-89 >emb\|CAA10321\| (AJ131206) |
| | | microbody NAD-dependent malate dehydrogenase |
| | | [Arabidopsis thaliana] Length = 354 |
| 368 | 2024368 | 5E-33 >sp\|O48609\|Y65L_HORVU YCF65-LIKE |
| | | PROTEIN PRECURSOR >gi \|2695931\|emb\|CAA10984\| |
| | | (AJ222779) hypothetical protein [Hordeum vulgare] |
| | | Length = 181 |
| 369 | 2024369 | 4E-14 >gb\|AAD39463.1\|AF135439_1 (AF135439) |
| | | formin binding protein 11 [Mus musculus] |
| | | Length = 953 |
| 370 | 2024370 | Tyr_Phospho_Site(495-502) |
| 371 | 2024371 | Pkc_Phospho_Site(19-21) |
| 372 | 2024372 | 3E-22 >sp\|P25070\|TCH2_ARATH CAL- |
| | | MODULIN-RELATED PROTEIN 2, |
| | | TOUCH-INDUCED >gi\|2583169 (AF026473) |
| | | calmodulin-related protein [Arabidopsis |
| | | thaliana] Length = 161 |
| 373 | 2024373 | 1E-142 >sp\|P35614\|ERF1_ARATH EUKARY- |
| | | OTIC PEPTIDE CHAIN RELEASE FACTOR |
| | | SUBUNIT 1 (ERF1) (OMNIPOTENT SUPPRESSOR |
| | | PROTEIN 1 HOMOLOG) (SUP1 |
| | | HOMOLOG) >gi\|322554\|pir\|\|S31328 omnipotent |
| | | suppressor protein SUP1 homolog (clone G18) - Arabidopsis |
| | | thaliana >gi\|16514\|emb\|CAA49172\| (X69375) |
| | | similar to yeast omnipotent suppressor protein SUP1 |
| | | (SUP45) [Arabidopsis |
| | | thaliana] >gi\|1402882\|emb\|CAA66813\| |
| | | (X98130) eukaryotic early release factor subunit 1-like |
| | | protein [Arabidopsis |
| | | thaliana] >gi\|1495249\|emb\|CAA66118\| |
| | | (X97486) eRF1-3 [Arabidopsis thaliana] |
| | | Length = 435 |
| 374 | 2024374 | 1E-1 11 >sp\|O04486\|RB1C_ARATH RAS- |
| | | RELATED PROTEIN RAB11C >gi\|2160157 |
| | | (AC000132) Strong similarity to A. thaliana ara-2 |
| | | (gb\|ATHARA2). ESTs |
| | | gb\|ATT52483,gb\|ATTS2484,gb\|AA042159 |
| | | come from this gene. [Arabidopsis |
| | | thaliana] >gi\|2231303 (U74669) ras-related small |
| | | GTPase [Arabidopsis thaliana] Length = 217 |
| 375 | 2024375 | 1E-53 >emb\|CAA74054\| (Y13726) Tran- |
| | | scription factor [Arabidopsis thaliana] Length = 155 |
| 376 | 2024376 | Tyr_Phospho_Site(1219-1225) |
| 377 | 2024377 | 1E-148 >pir\|\|FKMUA phytochrome A- |
| | | Arabidopsis thaliana >gi\|16421 \|emb\|CAA35221\| |
| | | (X17341) phyA photoreceptor [Arabidopsis thaliana] |
| | | Length = 1122 |
| 378 | 2024378 | Pkc_Phospho_Site(97-99) |
| 379 | 2024379 | 7E-89 >sp\|QO1474\|SARB_ARATH GTP-BINDING |
| | | PROTEIN SAR1B >gi\|322517\|pir\|\|528603 GTP- |
| | | binding protein - Arabidopsis thaliana >gi\|166734 |
| | | (M95795) GTP-binding protein [Arabidopsis thaliana] |
| | | Length = 193 |
| 380 | 2024380 | 3E-86 >sp\|P51412\|RK21_ARATH 50S |
| | | RIBOSOMAL PROTEIN L21, |
| | | CHLOROPLAST PRECURSOR |
| | | (CL21) >gi\|2129718\|pir\|\|S71282 ribosomal |
| | | protein L21 - Arabidopsis |
| | | thaliana >gi\|1149573\|emb\|CAA89887\| |
| | | (Z49787) chloroplast ribosomal large subunit protein |
| | | L21 [Arabidopsis thaliana] Length = 220 |
| 381 | 2024381 | Tyr_Phospho_Site(597-604) |
| 382 | 2024382 | 1E-20 >gb\|AAD46412.1\|AF096262_{‘3}1 (AF096262) |
| | | ER6 protein [Lycopersicon esculentum] |
| | | Length = 168 |
| 383 | 2024383 | 3E-69 >pir\|\|S51699 oleoyl-[acyl-carrier-protein] |
| | | hydrolase (EC 3.1.2.14) - Arabidopsis |
| | | thaliana >gi\|804948\|emb\|CAA85388\| (Z36911) |
| | | acyl-(acyl carrier protein) thioesterase [Arabidopsis |
| | | thaliana] Length = 412 |
| 384 | 2024384 | Tyr_Phospho_Site(1364-1370) |
| 385 | 2024385 | Tyr_Phospho_Site(321-328) |
| 386 | 2024386 | 1E-47 >gi\|2435522 (AF024504) contains |
| | | similarity to other AMP-binding enzymes |
| | | [Arabidopsis thaliana] Length = 480 |
| 387 | 2024387 | 2E-62 >emb\|CAA63012\| (X91919) LEA76 |
| | | homologue typel [Arabidopsis |
| | | thaliana] >gi\|5903037\|gb\|AAD55596.1\|AC008016_6 |
| | | (AC008016) Identical to gb\|X91919 LEA76 homologue |
| | | type1 from Arabidopsis thaliana. ESTs gb\|N97082, |
| | | gb\|Z27056 and gb\|Z29902 come from this gene. |
| | | Length = 169 |
| 388 | 2024388 | 3E-79 >gb\|AAD28242.1\|AF121355_1 (AF121355) |
| | | peroxiredoxin TPx1 [Arabidopsis thaliana] |
| | | Length = 162 |
| 389 | 2024389 | 7E-92 >gi\|3790567 (AF078821) RING-H2 |
| | | finger protein RHA1b [Arabidopsis thaliana] |
| | | Length = 157 |
| 390 | 2024390 | Tyr_Phospho_Site(726-733) |
| 391 | 2024391 | 1E-130 >emb\|CAA16760.1\| (AL021711) |
| | | nodulin-26-like protein [Arabidopsis |
| | | thaliana] Length = 241 |
| 392 | 2024392 | Pkc_Phospho_Site(20-22) |
| 393 | 20243936 | E-57 >gi\|1256595 (U38915) LytB |
| | | [Synechocystis PCC6803] Length = 379 |
| 394 | 2024394 | 1E-1 36 >gi\|3980396 (AC004561) C-4 sterol |
| | | methyl oxidase [Arabidopsis thaliana] |
| | | Length = 253 |
| 395 | 2024395 | Tyr_Phospho_Site(230-237) |
| 396 | 20243962 | E-62 >gi\|1353352 (U31975) alanine |
| | | aminotransferase [Chlamydomonas reinhardtii] |
| | | Length = 521 |
| 397 | 2024397 | 3E-57 >gi\|3004557 (AC003673) plasma |
| | | membrane proton pump H+ ATPase, PMA1 |
| | | [Arabidopsis thaliana] Length = 949 |
| 398 | 2024398 | 6E-81 ) >sp\|Q03509\|CAL6_ARATH CAL- |
| | | MODULIN-6 >gi\|1076298\|pir\|\|S35187 calmodulin 6 - |
| | | Arabidopsis thaliana >gi\|16227\|emb\|CAA78059\| |
| | | (Z12024) calmodulin [Arabidopsis thaliana] |
| | | Length = 149 |
| 399 | 2024399 | 8E-74 >dbj\|BAA74839\| (AB007801) |
| | | cytochrome b5 [Arabidopsis thaliana] |
| | | Length = 134 |
| 400 | 2024400 | Tyr_Phospho_Site(35-41) |
| 401 | 2024401 | Tyr_Phospho_Site(232-239) |
| 402 | 2024402 | 2E-1 3 >gb\|AAD15432\| (AC006218) non- |
| | | specific lipid-transfer protein precursor [Arabidopsis |
| | | thaliana] >gi\|4726121\|gb\|AAD2832.1\| |
| | | AC006436_12 (AC006436) nonspecific lipid-transfer |
| | | protein precursor [Arabidopsis thaliana] |
| | | Length = 169 |
| 403 | 2024403 | 3E-11 >gi\|4063742 (AC005851) phaseolin |
| | | G-box binding protein [Arabidopsis thaliana] |
| | | Length = 320 |
| 404 | 2024404 | IE-120 >gi\|4204697 (AF117063) inositol |
| | | polyphosphate 5-phosphatase At5P2 [Arabidopsis |
| | | thaliana] Length = 646 |
| 405 | 2024405 | 2E-63 >sp\|P20363 \|TBA3_ARATH TUBULIN |
| | | ALPHA-3/ALPHA-5 |
| | | CHAIN >gi\|99768\|pir\|\|A32712 |
| | | tubulin alpha-5 chain - Arabidopsis |
| | | thaliana >gi\|166912 (M17189) alpha-tubulin |
| | | [Arabidopsis thaliana] >gi\|166918 (M84698) |
| | | alpha-5 tubulin [Arabidopsis thaliana] Length = 450 |
| 406 | 2024406 | 1E-103 >sp\|P10796\|RBS2_ARATH RIBULOSE |
| | | BISPHOSPHATE CARBOXYLASE SMALL |
| | | CHAIN 1B PRECURSOR (RUBISCO SMALL |
| | | SUBUNIT 1B) >gi\|68062\|pir\|\|RKMUB1 |
| | | ribulose-bisphosphate carboxylase (EC 4.1.1.39) |
| | | small chain B1 precursor - Arabidopsis |
| | | thaliana >gi\|16193\|emb\|CAA32700\| |
| | | (X14564) ribulose bisphosphate carboxylase |
| | | [Arabidopsis thaliana] Length = 181 |
| 407 | 2024407 | 8E-17 >sp\|O02414\|DYL1_ANTCR DYNEIN |
| | | LIGHT CHAIN LC6, FLAGELLAR |
| | | OUTER ARM >gi\|2208914\|dbj\|BAA20525\| |
| | | (AB004830) outer arm dynein LC6 |
| | | [Anthocidaris crassispina] Length = 89 |
| 408 | 2024408 | 5E-70 >sp\|P32826\|CBPX_ARATH SERINE |
| | | CARBOXYPEPTIDASE PRECURSOR >gi\|166674 |
| | | (M81130) carboxypeptidase Y-like protein [Arabidopsis |
| | | thaliana] >gi\|445120\|prf\|\|1908426A |
| | | carboxypeptidase Y [Arabidopsis thaliana] |
| | | Length = 539 |
| 409 | 2024409 | 1E-125 >gi\|2288999 (AC002335) electron |
| | | transfer flavoprotein ubiquinone oxidoreductase |
| | | isolog [Arabidopsis thaliana] Length = 633 |
| 410 | 2024410 | 5E-71 >pir\|\|S71238 immunophilin |
| | | FKBP15-2 - Arabidopsis thaliana >gi\|1272408 |
| | | (U52047) immunophilin [Arabidopsis thaliana] |
| | | Length = 163 |
| 411 | 2024411 | Tyr_Phospho_Site(336-343) |
| 412 | 2024412 | Tyr_Phospho_Site(1253-1261) |
| 413 | 2024413 | 2E-83 >gb\|AAC78267.1\|AAC78267 (AC002330) |
| | | cullin-like 1 protein [Arabidopsis thaliana] |
| | | Length = 676 |
| 414 | 2024414 | Tyr_Phospho_Site(483-490) |
| 415 | 2024415 | 4E-70 >emb\|CAA68126\| (X99793) |
| | | induced upon wounding stress |
| | | [Arabidopsis thaliana] Length = 386 |
| 416 | 2024416 | 8E-85 >gb\|AAD24607.1\|AC005825_14 (AC005825) |
| | | ubiquitin-conjugating enzyme E2;GB:T21483 appears |
| | | to be a partially-spliced transcript from this gene |
| | | [Arabidopsis thaliana] Length = 148 |
| 417 | 2024417 | 7E-43 >dbj\|BAA74576\| (AB015906) |
| | | actin-related protein [Homo |
| | | sapiens] >gi\|5880496gb\|AAD54678.1\|AF041475_1 |
| | | (AF041475) BAF53b [Homo sapiens] Length = 426 |
| 418 | 2024418 | 1E-107 >emb\|CAB51064.1\| (AL096856) |
| | | betaine aldehyde dehydrogenase-like protein |
| | | [Arabidopsis thaliana] Length = 503 |
| 419 | 2024419 | 8E-20 >sp\|Q10425\|IF3X_SCHPO PROBABLE |
| | | EUKARYOTIC TRANSLATION |
| | | INITIATION FACTOR 3 SUBUNIT |
| | | 9 >gi\|1256531\|emb\|CAA94637\| (Z70691) |
| | | eukaryotic translation initiation factor 3 beta subunit |
| | | [Schizosaccharomyces pombe] Length = 725 |
| 420 | 2024420 | Pkc_Phospho_Site(39-41) |
| 421 | 2024421 | Pkc_Phospho_Site(12-14) |
| 422 | 2024422 | Tyr_Phospho_Site(781-788) |
| 423 | 2024423 | 1E-95 >gi\|1174162 (U44976) ubiquitin- |
| | | conjugating enzyme [Arabidopsis |
| | | thaliana] >gi\|3746915 (AF091106) |
| | | E2 ubiquitin-conjugating-like enzyme |
| | | [Arabidopsis thaliana] Length = 160 |
| 424 | 2024424 | Tyr_Phospho_Site(1 69-176) |
| 425 | 2024425 | 5E-85 ) >dbj\|BAA36336\| (AB015142) |
| | | AHP2 [Arabidopsis |
| | | thaliana] >gi\|4156241\|dbj\|BAA37110\| |
| | | (AB012568) ATHP1 [Arabidopsis thaliana] |
| | | Length = 156 |
| 426 | 2024426 | 1E-1 69 >sp\|Q43725\|CYSM_ARATH CYSTEINE |
| | | SYNTHASE, MITOCHONDRIAL PRECURSOR |
| | | (O-ACETYLSERINE SULFHYDRYLASE) |
| | | (O-ACETYLSERINE (THIOL)-LYASE) |
| | | (CSASE) >gi\|1488519\|emb\|CAA57498\| |
| | | (X81973) cysteine synthase [Arabidopsis thaliana] |
| | | Length = 424 |
| 427 | 2024427 | 1E-140 >gi\|2586125 (U89512) b-keto |
| | | acyl reductase [Arabidopsis |
| | | thaliana] Length = 253 |
| 428 | 2024428 | Tyr_Phospho_Site(247-254) |
| 429 | 2024429 | 8E-15 >emb\|CAB41927.1\| (AL049751) |
| | | ribosmal protein L13a like protein [Arabidopsis |
| | | thaliana] Length = 206 |
| 430 | 2024430 | Tyr_Phospho_Site(1028-1036) |
| 431 | 2024431 | 1E-108 >emb\|CAB41927.1\| (AL049751) |
| | | ribosomal protein L13a like protein |
| | | [Arabidopsis thaliana] Length = 206 |
| 432 | 2024432 | 3E-21 >gi\|3327394 (AC004483) |
| | | RNA helicase [Arabidopsis thaliana] |
| | | Length = 845 |
| 433 | 2024433 | 1E-105 >emb\|CAA18824.1\| (AL023094) |
| | | Nonclathrin coat protein gamma- like protein |
| | | [Arabidopsis thaliana] Length = 831 |
| 434 | 2024434 | Pkc_Phospho_Site(79-81) |
| 435 | 2024435 | 7E-98 >dbj\|BAA34687\| (AB016819) |
| | | UDP-glucose glucosyltransferase |
| | | [Arabidopsis thaliana] Length = 481 |
| 436 | 2024436 | 1E-99 ) >gi\|2342734 (AC002341) |
| | | DNA-binding protein isolog |
| | | [Arabidopsis thaliana] Length = 170 |
| 437 | 2024437 | 1E-Si >sp\|P12357\|PSAG_SPIOL PHOTO- |
| | | SYSTEM I REACTION CENTRE |
| | | SUBUNIT V PRECURSOR |
| | | (PHOTOSYSTEM 19 KD PROTEIN) |
| | | (PSI-G) >gi\|72686\|pir\|\|F1SP5 |
| | | photosystem I chain V precursor - |
| | | spinach >gi\|21299\|emb\|CAA315241 (X13134) |
| | | PSI subunit V p |
| 438 | 2024438 | Pkc_Phospho_Site(5-7) |
| 439 | 2024439 | 2E-44 >gi\|2558962 (AF025667) |
| | | histone H2B1 [Gossypium hirsutum] |
| | | Length = 147 |
| 440 | 2024440 | 3E-83 >emb\|CAB10419.1\| (Z97341) |
| | | transcription factor like protein |
| | | [Arabidopsis thaliana] Length = 467 |
| 441 | 2024441 | 4E-82 >gi\|3983125 (AF097648) |
| | | phosphate/triose-phosphate translocator |
| | | precursor [Arabidopsis thaliana] Length = 410 |
| 442 | 2024442 | Pkc_Phospho_Site(8-10) |
| 443 | 2024443 | Tyr_Phospho_Site(705-712) |
| 444 | 2024444 | Tyr_Phospho_Site(508-516) |
| 445 | 2024445 | 1E-52 >gb\|AAD03445.1\| (AF118223) |
| | | contains similarity to Helicobacter pylori |
| | | peptide methionine sulfoxide reductase (msrA) |
| | | (GB:AE000542) [Arabidopsis |
| | | thaliana] Length = 155 |
| 446 | 2024446 | 1E-133 >gi\|3482914 (AC003970) |
| | | Similar to nodulins and lipase |
| | | [Arabidopsis thaliana] Length = 370 |
| 447 | 2024447 | 2E-13 >sp\|P09970\|PSBI_TOBAC PHOTO- |
| | | SYSTEM II REACTION CENTER I |
| | | PROTEIN (PSII 4.8 KD |
| | | PROTEIN) >gi\|72694\|pir\|\|F2NTI |
| | | photosystem II protein psbI - common tobacco |
| | | chloroplast >gi\|81726\|pir\|\|S07877 |
| | | photosystem II protein psbI - white mustard |
| | | chloroplast >gi\|82558\|pir\|\|JN0315 |
| | | photosystem II protein psbI - rye |
| | | chloroplast >gi\|82631\|pir\|\|S01044 |
| | | photosystem II protein psbl - wheat |
| | | chloroplast >gi\|1363581\|pir\|\|S58S35 |
| | | photosystem II protein psbl - maize |
| | | chloroplast >gi\|12541\|emb\|CAA35617\| |
| | | (X17616) I polypeptide (AA 1-36) [Sinapis |
| | | alba] >gi\|14226\|emb\|CAA43849\| |
| | | (X61674) I protein [Secale |
| | | cereale] >gi\|14257\|emb\|CAA30562\| |
| | | (X07742) ORE 35 (AA 1-36) [Triticum |
| | | aestivum] >gi\|902205\|emb\|CAA60269\| |
| | | (X86563) PSII I protein [Zea |
| | | mays] >gi\|5881678\|dbj\|BAA84369.1\| |
| | | (AP000423) PSII I protein [Arabidopsis thaliana] |
| | | Length = 36 |
| 448 | 2024448 | 1E-126 >gi\|2642443 (AC002391) |
| | | cytochrome P450 [Arabidopsis |
| | | thaliana] Length = 516 |
| 449 | 2024449 | Tyr_Phospho_Site(1097-1103) |
| 450 | 2024450 | 5E-69 >sp\|P42798\|RS1A_ARATH 40S |
| | | RIBOSOMAL PROTEIN S15A >gi\|440824 |
| | | (L27461) ribosomal protein 515 [Arabidopsis |
| | | thaliana] >gi\|2150130 (AF001412) |
| | | cytoplasmic ribosomal protein S15a |
| | | [Arabidopsis thaliana] Length = 130 |
| 451 | 2024451 | Tyr_Phospho_Site(383-391) |
| 452 | 2024452 | Tyr_Phospho_Site(1136-1142) |
| 453 | 2024453 | Tyr_Phospho_Site(1164-1170) |
| 454 | 2024454 | 1E-1 16 >gb\|AAD24830.1\|AC007071_2 |
| | | (AC007071) RING finger protein |
| | | [Arabidopsis thaliana] Length = 565 |
| 455 | 2024455 | 1E-58 >gb\|AAD201381 (AC006282) |
| | | 60S ribosomal protein L24 [Arabidopsis |
| | | thaliana] >gi\|4581159\|gb\|AAD24643.1\|AC006919_21 |
| | | (AC006919) 60S ribosomal protein L24 |
| | | [Arabidopsis thaliana] Length = 177 |
| 456 | 2024456 | Tyr_Phospho_Site(194-201) |
| 457 | 2024457 | 1E-92 >pir\|\|S71217 glutamate |
| | | dehydrogenase 1 - Arabidopsis |
| | | thaliana >gi\|1098960 (U37771) glutamate |
| | | dehydrogenase 1 [Arabidopsis |
| | | thaliana] >gi\|1293095 (U53527) glutamate |
| | | dehydrogenase 1 [Arabidopsis thaliana] |
| | | Length = 411 |
| 458 | 2024458 | 4E-98 >gi\|3885328 (AC005623) |
| | | senine/threonine protein kinase |
| | | [Arabidopsis thaliana] Length = 441 |
| 459 | 2024459 | 2E-43 >gi\|3687237 (AC005169) |
| | | Cys3His zinc-finger protein [Arabidopsis |
| | | thaliana] Length = 359 |
| 460 | 2024460 | Tyr_Phospho_Site(862-868) |
| 461 | 2024461 | 8E-62 >gi\|3776558 (AC005388) |
| | | Identical to gb\|L14814 DNA for tissue- |
| | | specific acyl carrier protein isoform 2 from A. thaliana. |
| | | ESTs gb\|AA5973S1, gb\|T41805, gb\|H36871, |
| | | gb\|R30210, gb\|AA042549, gb\|Z47650, gb\|H76304 |
| | | and gb\|AA597348 come from this gene. [Arabidopsi... |
| | | Length = 136 |
| 462 | 2024462 | 2E-55 >gi\|3776561 (AC005388) |
| | | Identical to DNA for acyl carrier protein |
| | | (ACP) gene A2 gb\|X57699 from A. thaliana. |
| | | ESTs gb\|W43252, gb\|T42821, gb\|N65229, |
| | | gb\|N97267, gb\|F15491 and gb\|AA040955 |
| | | come from this gene. |
| | | [Arabidopsis thaliana] Length = 136 |
| 463 | 2024463 | 5E-82 >gi\|2668744 (AF034946) |
| | | ubiquitin conjugating enzyme [Zea mays] |
| | | Length = 148 |
| 464 | 2024464 | 2E-35 >gi\|4097569 (U64915) |
| | | GMFP4 [Glycine max] Length = 111 |
| 465 | 2024465 | Tyr_Phospho_Site(357-364) |
| 466 | 2024466 | 4E-30 >emb\|CAB39595.1\| (AL049480) |
| | | ribosomal protein Sb [Arabidopsis |
| | | thaliana] Length = 177 |
| 467 | 2024467 | 6E-99 ) >gb\|AAC26008.1\| (AF076251) |
| | | calcineurin B-like protein 1 |
| | | [Arabidopsis thaliana] Length = 213 |
| 468 | 2024468 | Pkc_Phospho_Site(40-42) |
| 469 | 2024469 | Tyr_Phospho_Site(125-132) |
| 470 | 2024470 | Zinc_Protease(124-133) |
| 471 | 2024471 | 1E-110 >sp\|O23654\|VATA_ARATH VACUOLAR |
| | | ATP SYNTHASE CATALYTIC |
| | | SUBUNIT A (V-ATPASE 69 KD |
| | | SUBUNIT) >gi\|2266990 (U65638) vacuolar type |
| | | ATPase subunit A [Arabidopsis |
| | | thaliana] >gi\|3834305 (AC005679) |
| | | Identical to gb\|U65638 Arabidopsis thaliana |
| | | vacuolar type ATPase subunit A mRNA. ESTs |
| | | gb\|N96435, gb\|N96106, gb\|N96189, gb\|N96091, |
| | | gb\|AA042286, gb\|F14324, gb\|W43643, gb\|N96027, |
| | | gb\|N96299, gb\|R29943, gb\|T43460, gb\|T43544, |
| | | gb\|T22472... Length = 623 |
| 472 | 2024472 | 3E-73 >sp\|Q42449\|PRO1_ARATH PROFILIN |
| | | 1 (ALLERGEN ARA T 8) >gi\|2981657\|pdb\|1AOK\| |
| | | Profilin I From Arabidopsis Thaliana >gi\|1353763 |
| | | (U43322) profilin 1 [Arabidopsis |
| | | thaliana] >gi\|1353770 (U43325) profilin 1 |
| | | [Arabidopsis thaliana] >gi\|1835878\|bbs\|179026 |
| | | (S82691) profilin isoform I [Arabidopsis thaliana, |
| | | Columbia, flowers, Peptide, 131 aa] [Arabidopsis |
| | | thaliana] >gi\|3687242 (AC005169) |
| | | profilin 1 [Arabidopsis thaliana] Length = 131 |
| 473 | 2024473 | 3E-13 >gb\|AAD15432\| (AC006218) |
| | | nonspecific lipid-transfer protein |
| | | precursor [Arabidopsis |
| | | thaliana] >gi\|4726121\|gb\|AAD28321.1\|AC006436_12 |
| | | (AC006436) nonspecific lipid-transfer protein precursor |
| | | [Arabidopsis thaliana] Length = 169 |
| 474 | 2024474 | 1E-117 >gb\|AAD14532\| (AC006200) |
| | | membrane transporter [Arabidopsis |
| | | thaliana] Length = 725 |
| 475 | 2024475 | 1E-102 >pir\|\|S51b69 amino acid transporter |
| | | AAP4 - Arabidopsis thaliana >gi\|608671 \|emb\|CAA54631\| |
| | | (X77500) amino acid transporter Arabidopsis |
| | | thaliana] Length = 466 |
| 476 | 2024476 | 4E-90 >gb\|AAD31589.1\|AC006922_21 (AC006922) |
| | | phenylalanine ammonia |
| | | lyase [Arabidopsis thaliana] Length = 725 |
| 477 | 2024477 | 2E-54 >emb\|CAA63223\| (X92491) TOM20 |
| | | [Solanum tuberosum] Length = 204 |
| 478 | 2024478 | Tyr_Phospho_Site(169-176) |
| 479 | 2024479 | Tyr_Phospho_Site(1199-1206) |
| 480 | 2024480 | Pkc_Phospho_Site(39-41) |
| 481 | 2024481 | Pkc_Phospho_Site(114-116) |
| 482 | 2024482 | Pkc_Phospho_Site(88-90) |
| 483 | 2024483 | Tyr_Phospho_Site(215-222) |
| 484 | 2024484 | 1E-100 >gi\|2829925 (AC002291) |
| | | Similar to dnaj-like protein, |
| | | gp\|Y11969\|2230757 [Arabidopsis |
| | | thaliana] Length = 351 |
| 485 | 2024485 | Tyr_Phospho_Site(652-659) |
| 486 | 2024486 | Pkc_Phospho_Site(69-71) |
| 487 | 2024487 | Tyr_Phospho_Site(1277-1284) |
| 488 | 2024488 | 2E-45 >emb\|CAA7O691\| (Y09482) |
| | | HMG1 [Arabidopsis |
| | | thaliana] >gi\|2832361 \|emb\|CAA74402.1\| |
| | | (Y14073) HMG protein [Arabidopsis thaliana] |
| | | Length = 141 |
| 489 | 2024489 | 6E-b 4 >sp\|P301851DH18_ARATH DEHYDRIN |
| | | RAB18 >gi\|282880\|pir\|\|S28021 rabi 8 protein - |
| | | Arabidopsis thaliana >gi\|16451 \|emb\|CAA48178\| |
| | | (X68042) RAB18 [Arabidopsis thaliana] |
| | | Length = 186 |
| 490 | 2024490 | 5E-37 >gi\|451193 (L28008) wali7 [Triticum |
| | | aestivum] >gi\|1090845\|prf\|\|2019486B |
| | | wali7 gene [Triticum aestivum] Length = 270 |
| 491 | 2024491 | Tyr_Phospho_Site(144-150) |
| 492 | 2024492 | 1E-85 >gi\|3860272 (AC005824) |
| | | suppressor protein [Arabidopsis |
| | | thaliana] >gi14314399\|gb\|AAD15609\| |
| | | (AC006232) skd1 protein [Arabidopsis |
| | | thaliana] Length = 435 |
| 493 | 2024493 | Tyr_Phospho_Site(1021-1028) |
| 494 | 2024494 | 1E-94 >emb\|CAA11858\| (AJ224161) |
| | | delta-8 sphingolipid desaturase |
| | | [Arabidopsis thaliana] Length = 449 |
| 495 | 2024495 | Tyr_Phospho_Site(1009-1016) |
| 496 | 2024496 | Tyr_Phospho_Site(62-69) |
| 497 | 2024497 | 1E-88 ) >gi\|3980405 (AC004561) tropinone |
| | | reductase [Arabidopsis thaliana] |
| | | Length = 262 |
| 498 | 2024498 | 9E-47 >sp\|P55852\|SMT3_ARATH UBIQUI- |
| | | TIN-LIKE PROTEIN |
| | | SMT3 >gi\|1707372\|emb\|CAA67923\| (X99609) |
| | | ubiquitin-like protein [Arabidopsis thaliana] |
| | | Length = 104 |
| 499 | 2024499 | 3E-11 >sp\|O04886\|PME1_CITSI PEC- |
| | | TINESTERASE 1.1 PRECURSOR |
| | | (PECTIN METHYLESTERASE) |
| | | (PE) >gi\|2098705 (U82973) pectinesterase |
| | | [Citrus sinensis] Length = 584 |
| 500 | 2024500 | 1E-66 >pir\|\|S58491 IAA3 |
| | | protein - Arabidopsis thaliana >gi\|972911 |
| | | (U18406) IAA3 [Arabidopsis |
| | | thaliana] >gi\|1903369\|gb\|AAB70452\| |
| | | (AC000104) Match to Arabidopsis IAA3 (gb\|U18406). |
| | | EST gb\|T04296 comes from this gene. |
| | | [Arabidopsis thaliana] Length = 189 |
| 501 | 2024501 | Rgd(875-877) |
| 502 | 2024502 | Tyr_Phospho_Site(43-50) |
| 503 | 2024503 | 1E-74 ) >sp\|P46313\|FD6E_ARATH OMEGA-6 |
| | | FATTY ACID DESATURASE, |
| | | ENDOPLASMIC RETICULUM (DELTA-12 |
| | | DESATURASE) >gi\|438451 (L26296) |
| | | delta-12 desaturase [Arabidopsis thaliana] |
| | | Length = 383 |
| 504 | 2024504 | 2E-88 ) >gi\|3785982 (AC005560) |
| | | major latex protein [Arabidopsis |
| | | thaliana] Length = 151 |
| 505 | 2024505 | Tyr_Phospho_Site(523-529) |
| 506 | 2024506 | Tyr_Phospho_Site(706-714) |
| 507 | 2024507 | 3E-80 >sp\|Q03510\|CAL4_ARATH CAL- |
| | | MODULIN-4 >gi\|479693\|pir\|\|535185 |
| | | calmodulin 4 - Arabidopsis |
| | | thaliana >gi\|16223\|emb[CAA78057\| |
| | | (Z12022) calmodulin [Arabidopsis thaliana] |
| | | Length = 149 |
| 508 | 2024508 | Tyr_Phospho_Site(156-162) |
| 509 | 2024509 | 1E-91 >gi\|2739389 (AC002505) |
| | | Cf-2.2 like protein [Arabidopsis thaliana] |
| | | Length = 480 |
| 510 | 2024510 | 1E-45 >gi\|3236242 (AC004684) 60S |
| | | ribosomal protein L36 [Arabidopsis |
| | | thaliana] Length = 113 |
| 511 | 2024511 | 8E-40 >emb\|CAA23O37.1\| (AL035394) |
| | | V-ATPase subunit G (vag2 gene) |
| | | [Arab idopsis thaliana] Length = 106 |
| 512 | 2024512 | Tyr_Phospho_Site(1042-1049) |
| 513 | 2024513 | Tyr_Phospho_Site(563-570) |
| 514 | 2024514 | 4E-50 >gi\|2829918 (AC002291) |
| | | similar to tub protein gp\|U82468\|2072162 |
| | | [Arabidopsis thaliana] Length = 455 |
| 515 | 2024515 | 1E-81 >emb\|CAB43885.1\| (AL078468) |
| | | acyl-CoA synthetase-like protein |
| | | [Arabidopsis thaliana] Length = 666 |
| 516 | 2024516 | Tyr_Phospho_Site(481-488) |
| 517 | 2024517 | Tyr_Phospho_Site(274-281) |
| 518 | 2024518 | Tyr_Phospho_Site(1095-1103) |
| 519 | 2024519 | Tyr_Phospho_Site(1315-1322) |
| 520 | 2024520 | Tyr_Phospho_Site(71-79) |
| 521 | 2024521 | 7E-31 >ref\|NP_003282.1\|PTPP2\| tripeptidyl |
| | | peptidase II >gi\|136107\|sp\|P29144\|TPP2_HUMAN |
| | | TRIPEPTIDYL-PEPTIDASE II (TPP II) |
| | | (TRIPEPTIDYL AMINOPEPTI- |
| | | DASE) >gi\|1082875\|pir\|\|S54376 tri- |
| | | peptidyl-peptidase II (EC 3.4.14.10) - |
| | | human >gi\|339880 (M73047) tripeptidyl peptidase II |
| | | [Homo sapiens] Length = 1249 |
| 522 | 2024522 | 1E-92 >pir\|\|S71252 lectin- |
| | | like protein - Arabidopsis |
| | | thaliana >gi\|995619\|emb\|CAA62665\| |
| | | (X91259) lectin like protein [Arabidopsis thaliana] |
| | | Length = 272 |
| 523 | 2024523 | 3E-25 >emb\|CAA16566\| (AL021635) |
| | | DNA binding protein [Arabidopsis |
| | | thaliana] Length = 324 |
| 524 | 2024524 | 8E-67 >sp\|Q38904\|PRO3_ARATH PROFILIN |
| | | 3 >gi\|1353765 (U43323) profilin |
| | | 3 [Arabidopsis thaliana] Length = 134 |
| 525 | 2024525 | 1E-38 >gi\|2565436 (AF028842) |
| | | DegP protease precursor [Arabidopsis |
| | | thaliana] Length = 437 |
| 526 | 2024526 | 2E-72 >emb\|CAB54877.1\| (AL117188) |
| | | ribosomal protein L11 homolog |
| | | [Arabidopsis thaliana] Length = 155 |
| 527 | 2024527 | 1E-28 >gb\|AAD55594.1\|AC008016_4 (AC008016) |
| | | Similar to gb\|D85381 cytochrome c oxidase subunit |
| | | Vb precursor from Oryza sativa. ESTs gb\|R30504 |
| | | and gb\|AA598195 come from this gene. |
| | | [Arabidopsis thaliana] Length = 102 |
| 528 | 2024528 | 1E-100 ) >gi\|1345132 (U47029) |
| | | ERECTA [Arabidopsis |
| | | thaliana] >gi\|1389566\|dbj\|BAA11869\| |
| | | (D83257) receptor protein kinase [Arabidopsis |
| | | thaliana] >gi\|3075386 (AC004484) |
| | | receptor protein kinase, ERECTA [Arabidopsis |
| | | thaliana |
| 529 | 2024529 | Pkc_Phospho_Site(241-243) |
| 530 | 2024530 | Tyr_Phospho_Site(913-921) |
| 531 | 2024531 | 6E-58 >gi\|4218987 (AF098630) |
| | | cell wall-plasma membrane disconnecting |
| | | CLCT protein [Arabidopsis |
| | | thaliana] >gi\|4725954\|emb\|CAB41725.1\| |
| | | (AL049730) cell wall-plasma membrane disconnecting |
| | | CLOT protein (AIR1A) |
| | | [Arabidopsis thaliana] Length = 111 |
| 532 | 2024532 | 6E-66 >emb\|CAA90703\| (Z50851) HD-zip |
| | | [Arabidopsis thaliana] Length = 833 |
| 533 | 2024533 | 6E-19 >ref\|NP_006214.1\|PPIN4\| protein |
| | | (peptidyl-prolyl cis/trans isomerase) |
| | | NIMA-interacting, 4 |
| | | (parvulin) >gi\|4689436\|gb\|AAD27893.1\|AF143096_1 |
| | | (AF143096) peptidyl-prolyl cis-trans isomerase EPVH |
| | | [Homo sapiens] >gi\|5420453\|dbj\|BAA82320.1\| |
| | | (AB009690) parvulin [Homo sapiens] Length |
| 534 | 2024534 | 1E-100 >emb\|CAB43O54.1\| (AL049876) |
| | | disease resistance response protein |
| | | [Arabidopsis thaliana] Length = 184 |
| 535 | 2024535 | 2E-51 >emb\|CAA73155\| 10 (Y12575) |
| | | histone H2A.F/Z [Arabidopsis thaliana] |
| | | Length = 136 |
| 536 | 2024536 | SE-83 >gi\|3176668 (AC004393) |
| | | Similar to ribosomal protein L17 |
| | | gb\|X62724 from Hordeum vulgare. ESTs |
| | | gb\|Z34728, gb\|F19974, gb\|T75677 and |
| | | gb\|Z33937 come from this gene. |
| | | [Arabidopsis thaliana] Length = 175 |
| 537 | 2024537 | Tyr_Phospho_Site(443-450) |
| 538 | 2024538 | 2E-41 >emb\|CAA90663.1\| (Z50795) |
| | | weak similarity with yeast cat8 regulatory protein |
| | | (Swiss Prot accession number P39113); cDNA EST |
| | | EMBL:Z14554 comes from this gene; cDNA EST |
| | | EMBL:T02057 comes from this gene; cDNA EST |
| | | EMBL:D75504 comes from this gene... Length = 618 |
| 539 | 2024539 | Tyr_Phospho_Site(448-454) |
| 540 | 2024540 | 2E-26 >sp\|P72874\|IF3SYNY3 TRANSLATION |
| | | INITIATION FACTOR |
| | | IF-3 >gi\|1651964\|dbj\|BAA168901 (D90901) |
| | | initiation factor IF-3 [Synechocystis sp.] |
| | | Length = 177 |
| 541 | 2024541 | 1E-104 >emb\|CAA73792\| (Y13356) |
| | | glyoxysomal isocitrate lyase [Brassica |
| | | napus] Length = 576 |
| 542 | 2024542 | 9E-76 >gb\|AAD51109.1\|AF176040_1 |
| | | (AF176040) ubiquitin-conjugating enzyme |
| | | UBC2 [Mesembryanthemum crystallinum] Length 148 |
| 543 | 2024543 | Tyr_Phospho_Site(917-925) |
| 544 | 2024544 | Tyr_Phospho_Site(1401-1407) |
| 545 | 2024545 | Pkc_Phospho_Site(25-27) |
| 546 | 2024546 | 9E-75 >emb\|CAA76606\| (Y17053) |
| | | At-heat shock 70-3 protein, |
| | | [Arabidopsis thaliana] Length = 649 |
| 547 | 2024547 | 1E-107 >gi\|3287687 (AC003979) |
| | | Match to sucrose-proton symporter |
| | | (SUC2) gene gb\|X75382 from A. thaliana. |
| | | [Arabidopsis thaliana] Length = 512 |
| 548 | 2024548 | 3E-42 >gi\|3065814 (AF058714) |
| | | sodium-dicarboxylate cotransporter |
| | | SDCT1 [Rattus |
| | | norvegicus] >gi\|3168585\|dbj\|BAA28609\| |
| | | (AB001321) sodium-dependent dicarboxylate transporter |
| | | [Rattus norvegicusi Length = 587 |
| 549 | 2024549 | 3E-18 >sp\|O35075\|DCRA_MOUSE DOWN |
| | | SYNDROME CRITICAL REGION |
| | | PROTEIN A >gi\|2588993\|dbj\|BAA23270\| |
| | | (AB001990) Dcra [Mus musculus] Length = 297 |
| 550 | 2024550 | 4E-88 >gi\|166834 (M86720) ribulose |
| | | bisphosphate carboxylase/oxygenase activase |
| | | [Arabidopsis thaliana] >gi\|2642155 |
| | | (AC003000) Rubisco activase [Arabidopsis thaliana] |
| | | Length = 474 |
| 551 | 2024551 | 2E-72 >pir\|\|S71209 ubiquitin conjugating enzyme |
| | | E2 protein - Arabidopsis thaliana >gi\|992704 |
| | | (U33757) UBC7 [Arabidopsis thaliana] |
| | | Length = 166 |
| 552 | 2024552 | 2E-58 >emb\|CAA23033.1\| (AL035394) |
| | | major latex protein [Arabidopsis |
| | | thaliana] Length = 151 |
| 553 | 2024553 | Tyr_Phospho_Site(883-890) |
| 554 | 2024554 | 3E-36 >gi\|3033377 (AC004238) |
| | | berberine bridge enzyme [Arabidopsis |
| | | thaliana] Length = 540 |
| 555 | 2024555 | 3E-24 >emb\|CAA15173\| (AJ235273) |
| | | GLUTAREDOXIN-LIKE PROTEIN |
| | | GRLA (grxC2) [Rickettsia prowazekii] Length = 107 |
| 556 | 2024556 | 6E-53 >emb\|CAA23022.1\| (AL035394) |
| | | cellulase [Arabidopsis thaliana] |
| | | Length = 479 |
| 557 | 2024557 | 8E-54 >emb\|CAA16683\| (AL021684) |
| | | lysosomal Pro-X carboxypeptidase - |
| | | like protein [Arabidopsis thaliana] Length = 499 |
| 558 | 2024558 | Tyr_Phospho_Site(416-424) |
| 559 | 2024559 | Pkc_Phospho_Site(101-103) |
| 560 | 2024560 | Pkc_Phospho_Site(22-24) |
| 561 | 2024561 | 2E-21 >gi\|3834319 (AC005679) |
| | | Similar to gi\|2244754 heat shock transcription |
| | | factor HSF30 homolog from Arabidopsis thaliana |
| | | chromosome 4 contig gb\|Z97335. [Arabidopsis |
| | | thaliana] Length = 458 |
| 562 | 2024562 | 1E-50 >emb\|CAA22574.1\| (AL034567) |
| | | ubiquinol-cytochrome c reductase-like protein |
| | | [Arabidopsis thaliana] Length = 122 |
| 563 | 2024563 | 3E-87 >gi\|1732570 (U72153) beta- |
| | | glucosidase [Arabidopsis thaliana] |
| | | Length = 525 |
| 564 | 2024564 | Tyr_Phospho_Site(669-676) |
| 565 | 2024565 | Tyr_Phospho_Site(399-406) |
| 566 | 2024566 | Pkc_Phospho_Site(31-33) |
| 567 | 2024567 | Rgd(900-902) |
| 568 | 2024568 | 2E-58 >emb\|CAA06925\| (AJ006228) |
| | | Avr9 elicitor response protein |
| | | [Nicotiana tabacum] Length = 396 |
| 569 | 2024569 | Pkc_Phospho_Site(8-10) |
| 570 | 2024570 | 2E-74 >sp\|Q96558\|UGDH_SOYBN UDP- |
| | | GLUCOSE 6-DEHYDROGENASE (UDP- |
| | | GLC DEHYDROGENASE) (UDP-GLCDH) |
| | | (UDPGDH) >gi\|1518540 (U53418) UDP- |
| | | glucose dehydrogenase [Glycine max] |
| | | Length = 480 |
| 571 | 2024571 | 1E-64 >gb\|AAD15398\| (AC006223) |
| | | ribosomal protein S12 [Arabidopsis |
| | | thaliana] Length = 144 |
| 572 | 2024572 | 1E-45 >gi\|3142301 (AC002411) |
| | | Contains similarity to neural cell adhesion |
| | | molecule 2, large isoform precursor gb\|M76710 |
| | | from Xenopus laevis, and beta transducin from |
| | | S. cerevisiae gb\|Q05946. ESTs gb\|N65081 |
| | | gb\|Z30910, gb\|Z34190, gb\|Z34611, gb\|R30101, |
| | | gb\|H3630... Length = 838 |
| 573 | 2024573 | Tyr_Phospho_Site(639-647) |
| 574 | 2024574 | Pkc_Phospho_Site(184-186) |
| 575 | 2024575 | Zinc_Protease(1049-1058) |
| 576 | 2024576 | 3E-58 >emb\|CAA63482\| (X92888) |
| | | glycolate oxidase [Lycopersicon esculentum] |
| | | Length = 290 |
| 577 | 2024577 | Pkc_Phospho_Site(84-86) |
| 578 | 2024578 | Tyr_Phospho_Site(1378-1384) |
| 579 | 2024579 | 2E-39 >gi\|3980417 (AC004561) |
| | | pumilio-like protein [Arabidopsis |
| | | thaliana] Length = 964 |
| 580 | 2024580 | 5E-75 >gb\|AAC34215.1\| (AC004411) |
| | | anion exchange protein 3 [Arabidopsis thaliana] |
| | | Length = 344 |
| 581 | 2024581 | 2E-80 ) >gb\|AAC78255.1\|AAC78255 (AC002330) |
| | | bZIP-Iike DNA binding protein |
| | | [Arabidopsis thaliana] Length = 411 |
| 582 | 2024582 | 4E-47 >emb\|CAB16844.1\| (Z99708) serine |
| | | O-palmitoyltransferase like protein |
| | | [Arabidopsis thaliana] Length = 475 |
| 583 | 2024583 | 2E-18 >gb\|AAD20711\| (AC006300) |
| | | phosphate/phosphoenolpyruvate translocator |
| | | protein [Arabidopsis thaliana] Length = 347 |
| 584 | 2024584 | 3E-20 >gb\|AAD46141.1\|AF081022_1 |
| | | (AF081022) hypoxia-induced protein L31 |
| | | [Lycopersicon esculentum] Length = 78 |
| 585 | 2024585 | 3E-47 >gb\|AAD46410.1\|AF096260_1 |
| | | (AF096260) ER66 protein [Lycopersicon |
| | | esculentum] Length = 558 |
| 586 | 2024586 | Tyr_Phospho_Site(304-310) |
| 587 | 2024587 | Tyr_Phospho_Site(954-961) |
| 588 | 2024588 | SE-69 >emb\|CAA52772\| (X74756) |
| | | ATAF2 [Arabidopsis thaliana] Length = 214 |
| 589 | 2024589 | 2E-64 >sp\|P21653\|TIP1_TOBAC |
| | | TONOPLAST INTRINSIC PROTEIN, |
| | | ROOT-SPECIFIC RB7-5A |
| | | (RT-TIP) >gi\|82192\|pir\|\|JQ1011 |
| | | TobRB7-5A protein - common |
| | | tobacco >gi\|100371\|pir\|\|S13719 probable |
| | | membrane channel protein - common |
| | | tobacco >gi\|20011\|emb\|CAA38634\| (X54855) |
| | | possible membrane channel protein |
| | | [Nicotiana tabacum] Length = 250 |
| 590 | 2024590 | Tyr_Phospho_Site(11-19) |
| 591 | 2024591 | Tyr_Phospho_Site(842-849) |
| S92 | 2024592 | 1E-75 >gi\|3582341 (AC005496) |
| | | flavonol 3-o-glucosyltransferase |
| | | [Arabidopsis thaliana] Length = 474 |
| 593 | 2024593 | 5E-16 >gb\|AAC97956.1\| (AF103731) |
| | | glycolipid transfer protein [Homo |
| | | sapiens] Length = 391 |
| 594 | 2024594 | 1E-30 >gb\|AAD56411.1\|AF185269_1 |
| | | (AF185269) bHLH transcription factor |
| | | GBOF-1 [Tulipa gesneriana] Length = 321 |
| 595 | 2024595 | Pkc_Phospho_Site(48-50) |
| 596 | 2024596 | 1E-39 >gi\|1946362 (U93215) |
| | | photosystem II reaction center 6.1KD |
| | | protein [Arabidopsis thaliana] Length = 133 |
| 597 | 2024597 | Rgd(1271-1273) |
| 598 | 2024598 | 1E-1 01 >emb\|CAA72363\| (Y11650) |
| | | cyclic phosphodiesterase [Arabidopsis |
| | | thaliana] >gi\|2832621 \|emb\|CAA16750.1\| |
| | | (AL021711) cyclic phosphodiesterase |
| | | [Arabidopsis thaliana] Length = 181 |
| 599 | 2024599 | Tyr_Phospho_Site(1199-1207) |
| 600 | 2024600 | 3E-26 >gb\|AAC78298.1\| (AF054615) |
| | | cellulase [Fragaria x ananassa] Length = 561 |
| 601 | 2024601 | 7E-59 >ref\|NP_001016.1\|PRPS23\| ribosomal |
| | | protein S23 >gi\|730647\|sp\|P39028\|RS23_HUMAN |
| | | 40S RIBOSOMAL PROTEIN |
| | | S23 >gi\|543449\|pir\|\|541955 ribosomal protein |
| | | S23 - rat >gi\|631360\|pir\|\|S42105 |
| | | ribosomal protein S23, cytosolic - human >gi |
| 602 | 2024602 | 4E-60 >emb\|CAA18139\| (AL022141) |
| | | cytochrome P450 like protein (fragment) |
| | | [Arabidopsis thaliana] Length = 255 |
| 603 | 2024603 | 3E-67 ) >sp\|P48578\|P2A4_ARATH SERINE/ |
| | | THREONINE PROTEIN |
| | | PHOSPHATASE PP2A-4 CATALYTIC |
| | | SUBUNIT >gi\|2117984\|pir\|\|552660 |
| | | phosphoprotein phosphatase (EC 3.1.3.16) 2A isoform |
| | | 4 - Arabidopsis thaliana >gi\|473259 |
| | | (U08047) Ser/Thr protei |
| 604 | 2024604 | 8E-64 ) >gb\|AAD56997.1\|AC009465_11 |
| | | (AC009465) ribosomal protein s19 or |
| | | s24 [Arabidopsis thaliana] Length = 133 |
| 605 | 2024605 | 2E-67 >gb\|AAD40139.1\|AF149413_20 |
| | | (AF149413) similar to malate dehydrogenases; |
| | | Pfam PF00390, Score=1290.5.E=0, N=1 |
| | | [Arabidopsis thaliana] Length = 588 |
| 606 | 2024606 | 2E-16 >ref\|NP_005099.1\|PXYLB\| xylulokinase |
| | | (H. influenzae) homolog >gi\|3298502\|dbj\|BAA31527\| |
| | | (AB015046) xylulokinase [Homo sapiens] |
| | | Length = 527 |
| 607 | 2024607 | Tyr_Phospho_Site(111-118) |
| 608 | 2024608 | 3E-65 ) >dbj\|BAA82063.1\| (AB022325) |
| | | pClpP [Arabidopsis |
| | | thaliana] >gi\|5881719\|dbj\|BAA84410.1\| |
| | | (AP000423) ATP-dependent protease subunit |
| | | [Arabidopsis thaliana] Length = 196 |
| 609 | 2024609 | 2E-35 >emb\|CAA05875\| (AJ003119) |
| | | protein phosphatase 20 [Arabidopsis |
| | | thaliana] Length = 511 |
| 610 | 2024610 | Tyr_Phospho_Site(469-475) |
| 611 | 2024611 | 1E-1 17 >gi\|2827139 (AF027172) |
| | | cellulose synthase catalytic subunit [Arabidopsis |
| | | thaliana] >gi\|4049343\|emb\|CAA22568.1\| |
| | | (AL034567) cellulose synthase catalytic subunit (RSW1) |
| | | [Arabidopsis thaliana] Length = 1081 |
| 612 | 2024612 | 1E-86 >pir\|\|S61555 xyloglucan endo-transglycosylase |
| | | precursor-Arabidopsis |
| | | thaliana >gi\|944810\|dbj\|BAA09783\| |
| | | (D63508) endo-xyloglucan transferase [Arabidopsis |
| | | thaliana] >gi\|5730137\|emb\|CAB52471.1\| |
| | | (AL109796) xyloglucan endo-1,4-beta-D-glucanase |
| | | precursor [Arabidopsis thaliana] Length = 269 |
| 613 | 2024613 | 5E-22 >gi\|4164473 (AF061157) |
| | | negatively light-regulated protein |
| | | [Vernicia fordii] Length = 108 |
| 614 | 2024614 | 4E-63 >gi\|2462840 (AF000657) |
| | | cytochrome C [Arabidopsis thaliana] |
| | | Length = 114 |
| 615 | 2024615 | 1E-100 >pir\|\|S62783 UDPglucose |
| | | 4-epimerase (EC 5.1.3.2) - Arabidopsis |
| | | thaliana >gi\|1143392\|emb\|CAA90941\| |
| | | (Z54214) uridine diphosphate glucose epimerase |
| | | [Arabidopsis thaliana] Length = 351 |
| 616 | 2024616 | Pkc_Phospho_Site(5-7) |
| 617 | 2024617 | Tyr_Phospho_Site(852-859) |
| 618 | 2024618 | 1E-61 >gi\|3319355 (AF077407) |
| | | similar to chaperonin containing TCP-1 |
| | | complex gamma chain [Arabidopsis |
| | | thaliana] Length = 562 |
| 619 | 2024619 | 4E-45 >gb\|AAD18030 (AF118129) |
| | | Tsi1-interacting protein TSIP1 |
| | | [Nicotiana tabacum] Length 154 |
| 620 | 2024620 | 1E-105 >emb\|CAA69258\| (Y07961) |
| | | GOP-associated inhibitor [Arabidopsis |
| | | thaliana] Length = 445 |
| 621 | 2024621 | 5E-96 >gi\|2462758 (AC002292) |
| | | RNA-binding protein [Arabidopsis |
| | | thaliana] Length = 292 |
| 622 | 2024622 | 5E-99 >gb\|AAD14467\| (AC005275) |
| | | LRR receptor-linked protein kinase |
| | | [Arabidopsis thaliana] Length = 754 |
| 623 | 2024623 | 6E-40 >emb\|CAB10346.1\| (Z97339) |
| | | glutaredoxin [Arabidopsis thaliana] |
| | | Length = 102 |
| 624 | 2024624 | 4E-35 >gi\|2982283 (AF051226) |
| | | PREG-like protein [Picea mariana] |
| | | Length = 284 |
| 625 | 2024625 | 1E-125 >pir\|\|S58497 IAA11 |
| | | protein - Arabidopsis thaliana >gi\|972925 |
| | | (U18413) IAA11 [Arabidopsis thaliana] Length = 246 |
| 626 | 2024626 | 1E-74 >gb\|AAD31363.1\|AC006053_5 |
| | | (AC006053) proton-ATPase-like protein |
| | | [Arabidopsis thaliana] Length = 178 |
| 627 | 2024627 | 2E-36 >sp\|P04465\|CALM_TRYBB |
| | | CALMODULIN >gi\|71679\|pir\|\|MCUTG |
| | | calmodulin - Trypanosoma brucel |
| | | gambiense >gi\|539401\|pir\|\|A48111 |
| | | calmodulin C - Trypanosoma |
| | | brucei >gi\|10386\|emb\|CAA39861\| (X56511) |
| | | calmodulin [Trypanosoma brucei] Length = 149 |
| 628 | 2024628 | 3E-98 >emb\|CAB42911.1\| (AL049862) |
| | | protein 1 photosystem II oxygen- |
| | | evolving complex [Arabidopsis |
| | | thaliana] >gi\|5748502 \|emb\|CAB53092.1\| |
| | | (AJ145957) precursor of the 33 kDa subunit |
| | | of the oxygen evolving complex |
| | | [Arabidopsis thaliana] Length = 331 |
| 629 | 2024629 | Tyr_Phospho_Site(101-108) |
| 630 | 2024630 | 2E-34 >gb\|AAD25756.1\|AC007060_14 |
| | | (AC007060) Contains the PF\|00650 |
| | | CRAL/TRIO phosphatidyl-inositol-transfer |
| | | protein domain. ESTs gb\|T76582, gb\|N06574 |
| | | and gb\|Z25700 come from this gene. |
| | | [Arabidopsis thaliana] Length = 540 |
| 631 | 2024631 | 9E-82 >emb\|CAB38268\| (AL035602) |
| | | UDP rhamnose-anthocyanid in-3-glucoside |
| | | rhamnosyltransferase-like protein |
| | | [Arabidopsis thaliana] Length = 455 |
| 632 | 2024632 | 2E-91 >gb\|AAD5S461.1\|AC009322_1 |
| | | (AC009322) Heat-shock protein |
| | | [Arabidopsis thaliana] Length = 831 |
| 633 | 2024633 | 1E-56 >pir\|\|S51478 drought-induced |
| | | protein Di19 - Arabidopsis |
| | | thaliana >gi\|469110\|emb\|CAA55321\| |
| | | (X78584) Di19 [Arabidopsis thaliana] |
| | | Length = 206 |
| 634 | 2024634 | Pkc_Phospho_Site(50-52) |
| 635 | 2024635 | Tyr_Phospho_Site(1341-1349) |
| 636 | 2024636 | 1E-102 >sp\|Q05153\|SSRP_ARATH STRUCTURE- |
| | | SPECIFIC RECOGNITION |
| | | PROTEIN 1 HOMOLOG (HMG |
| | | PROTEIN) >gi\|217853\|dbj\|BAA02719\| |
| | | (D13491) high mobility group protein |
| | | [Arabidopsis thaliana] Length = 644 |
| 637 | 2024637 | 2E-50 >gi\|1922944 (AC000106) |
| | | Strong similarity to Picea histone H2A (gb\|X67819). |
| | | ESTs gb\|ATTS3874,gb\|T46627,gb\|T14194 |
| | | come from this gene. |
| | | [Arabidopsis thaliana] Length = 142 |
| 638 | 2024638 | 2E-61 >dbj\|BAA13947\| (D89341) |
| | | luminal binding protein [Arabidopsis |
| | | thaliana] Length = 669 |
| 639 | 2024639 | 2E-51 >emb\|CAA19574.1\| (AL023859) |
| | | SPBC19C7.06, prolyl-trna synthetase, Ien:71 6aa, |
| | | similar eg. to YHR020W YHI0_YEAST, |
| | | P38708, p rolyl-trna synthetase yhr02, (688aa), |
| | | fasta scores, opt:248 6, EO:0, (55.1% identity in |
| | | 682 aa overl... Length = 716 |
| 640 | 2024640 | 8E-19 >gi\|3953473 (AC002328) |
| | | F2202.18 [Arabidopsis |
| | | thaliana] >gi\|5734520\|emb\|CAB52748.1\| |
| | | (AJ245630) photosystem I subunit V precursor |
| | | [Arabidopsis thaliana] Length 160 |
| 641 | 2024641 | 1E-85 ) >sp\|P35132\|UBC9_ARATH UBIQUITIN- |
| | | CONJUGATING ENZYME E2-17 KD 9 |
| | | (UBIQUITIN-PROTEIN LIGASE 9) |
| | | (UBIQUITIN CARRIER PROTEIN 9) |
| | | (UBCAT4B) >gi\|421857\|pir\|\|S32674 |
| | | ubiquitin-protein ligase (EC 6.3.2.19) UBC9 - |
| | | Arabidopsis thalia |
| 642 | 2024642 | Pkc_Phospho_Site(10-12) |
| 643 | 2024643 | 3E-84 >emb\|CAA18217.1\| (AL022223) |
| | | fructose-bisphosphate aldolase-like |
| | | protein [Arabidopsis thaliana] Length = 358 |
| 644 | 2024644 | 4E-86 ) >dbj\|BAA34251\| (AB013887) |
| | | RAV2 [Arabidopsis thaliana] Length = 352 |
| 645 | 2024645 | 3E-68 >dbj\|BAA31512\| (AB010880) |
| | | chloroplast ribosomal protein L17 |
| | | [Nicotiana tabacum] Length = 205 |
| 646 | 2024646 | 1E-67 >dbj\|BAA08282\| (D45848) |
| | | calmodulin-related protein [Arabidopsis |
| | | thaliana] >gi\|3402707 (AC004261) |
| | | calmodulin-related protein [Arabidopsis |
| | | thaliana] Length = 324 |
| 647 | 2024647 | 6E-87 >sp\|P24704\|SODC_ARATH SUPER- |
| | | OXIDE DISMUTASE |
| | | [CU-ZN] >gi\|66372\|pir\|\|DSMUZ superoxide |
| | | dismutase (EC 1.15.1.1) (Cu-Zn) - Arabidopsis |
| | | thaliana >gi\|16250\|emb\|CAA43270\| (X60935) |
| | | superoxide dismutase [Arabidopsis |
| | | thaliana] Length = 152 |
| 648 | 2024648 | 7E-64 >sp\|Q42S89\|NLT1_ARATH NON- |
| | | SPECIFIC LIPID-TRANSFER |
| | | PROTEIN 1 PRECURSOR (LTP |
| | | 1) >gi\|177796 (M80567) non-specific lipid |
| | | transfer protein [Arabidopsis |
| | | thaliana] >gi\|3786018 (AC005499) |
| | | unknown protein |
| | | [Arabidopsis thaliana] Length = 118 |
| 649 | 2024649 | 8E-31 >gi\|3859560 (AF098668) |
| | | acyl-protein thioesterase [Homo |
| | | sapiens] >gi\|4581413\|emb\|CAB40158.1\| |
| | | (AL031295) dJ886K2.4 (acyl-protein thioesterase) |
| | | [Homo sapiens] Length = 231 |
| 650 | 2024650 | 4E-97 ) >sp\|P46309\|GSH1_ARATH GLU- |
| | | TAMATE-CYSTEINE LIGASE |
| | | PRECURSOR (GAMMA-GLUTAMYLCYSTEINE |
| | | SYNTHETASE) (GAMMA-ECS) |
| | | (GCS) >gi\|2129598\|pir\|\|S60128 glutamate- |
| | | cysteine ligase (EC 6.3.2.2) precursor, |
| | | chloroplast - Arabidopsis thali |
| 651 | 2024651 | Tyr_Phospho_Site(1134-1141) |
| 652 | 2024652 | 2E-76 >sp\|P42733\|R11B_ARATH 40S |
| | | RIBOSOMAL PROTEIN S11-BETA >gi\|166869 |
| | | (L07877) ribosomal protein S11 |
| | | [Arabidopsis thaliana] Length = 159 |
| 653 | 2024653 | 8E-89 ) >sp\|P29511\|TBA6_ARATH TUBULIN |
| | | ALPHA-6 CHAIN >gi\|282852\|pir\|\|JQ1597 |
| | | tubulin alpha-6 chain - Arabidopsis |
| | | thaliana >gi\|166920 (M84699) TUA6 [Arabidopsis |
| | | thaliana] >gi\|2244853\|emb\|CAB10275.1\| |
| | | (Z97337) tubulin alpha-6 chain (TUA6) |
| | | [Arabidopsis thaliana] Length = 450 |
| 654 | 2024654 | 2E-48 >gi\|2982283 (AF051226) |
| | | PREG-like protein [Picea mariana] |
| | | Length = 284 |
| 655 | 2024655 | 1E-180 ) >gb\|AAD34236.1\|AF083913_1 |
| | | (AF083913) annexin [Arabidopsis |
| | | thaliana] Length = 317 |
| 656 | 2024656 | 1E-108 >emb\|CAA07574.1\| (AJ007587) |
| | | monooxygenase [Arabidopsis |
| | | thaliana] Length = 397 |
| 657 | 2024657 | 4E-18 >gi\|1913901 (U90919) |
| | | zinc finger protein [Homo sapiens] |
| | | Length = 478 |
| 658 | 2024658 | 2E-35 >gb\|AAD46413.1\|AF096263_1 |
| | | (AF096263) ER33 protein [Lycopersicon |
| | | esculentum] Length = 140 |
| 659 | 2024659 | Pkc_Phospho_Site(26-28) |
| 660 | 2024660 | 6E-87 >sp\|P40229\|KC2C_ARATH CASEIN |
| | | KINASE II BETA'CHAIN |
| | | (CK II) >gi\|1076300\|pir\|\|S47968 |
| | | casein kinase II (EC 2.7.1.-) beta chain CKB2 - |
| | | Arabidopsis thaliana >gi\|467975 (U03984) |
| | | casein kinase II beta subunit CKB2 [Arabidopsis |
| | | thaliana] >gi\|2245l22\|emb\|CAB10544.1\| |
| | | (Z97343) casein kinase II beta chain CKB2 |
| | | [Arabidopsis thaliana] Length = 282 |
| 661 | 2024661 | Pkc_Phospho_Site(11-13) |
| 662 | 2024662 | Tyr_Phospho_Site(272-279) |
| 663 | 2024663 | 1E-16 >sp\|Q6262S\|MPL3_RAT MICRO- |
| | | TUBULE-ASSOCIATED PROTEINS |
| | | 1A/1B LIGHT CHAIN 3 (MAP1A/MAP1B |
| | | LC3) >gi\|1083715\|pir\|\|A53624 |
| | | microtubule-associated protein 1 light chain 3 - |
| | | rat >gi\|455109 (U05784) light chain |
| | | 3 subunit of microtubule-associated proteins 1A and 1B |
| | | [Rattus norvegicus] Length = 142 |
| 664 | 2024664 | Pkc_Phospho_Site(122-124) |
| 665 | 2024665 | 2E-61 >emb\|CAA16688\| (AL021684) |
| | | receptor protein kinase - like protein |
| | | [Arabidopsis thaliana] Length = 1003 |
| 666 | 2024666 | 4E-42 >gi\|2275217 (AC002337) |
| | | chloroplast protein CP12 isolog |
| | | [Arabidopsis thaliana] Length = 124 |
| 667 | 2024667 | Rgd(1000-1002) |
| 668 | 2024668 | Tyr_Phospho_Site(295-301) |
| 669 | 2024669 | Pkc_Phospho_Site(19-21) |
| 670 | 2024670 | 1E-102 ) >emb\|CAA18217.1\| (AL022223) |
| | | fructose-bisphosphate aldolase-like protein |
| | | [Arabidopsis thaliana] Length = 358 |
| 671 | 2024671 | 5E-15 >gb\|AAD17407\| (AC006248) |
| | | salt-inducible protein [Arabidopsis |
| | | thaliana] Length = 627 |
| 672 | 2024672 | 9E-11 >gb\|AAD09328.1\| (AF019082) |
| | | virulent strain associated lipoprotein |
| | | [Borrelia burgdorferi] Length = 460 |
| 673 | 2024673 | 2E-79 >gi\|3927837 (AC005727) |
| | | core protein [Arabidopsis thaliana] |
| | | Length = 148 |
| 674 | 2024674 | Pkc_Phospho_Site(14-16) |
| 675 | 2024675 | 5E-87 >gi\|3128228 (AC004077) |
| | | ribosomal protein L18A [Arabidopsis |
| | | thaliana] >gi\|3337376 (AC004481) |
| | | ribosomal protein Li 8A [Arabidopsis thaliana] |
| | | Length = 178 |
| 676 | 2024676 | 1E-64 >gi\|2102691 (U64817) |
| | | fructokinase [Lycopersicon esculentum] |
| | | Length = 347 |
| 677 | 2024677 | 2E-90 >sp\|P10797\|RBS3_ARATH RIBULOSE |
| | | BISPHOSPHATE CARBOXYLASE SMALL |
| | | CHAIN 2B PRECURSOR (RUBISCO SMALL |
| | | SUBUNIT 2B) >gi\|68061\|pir\|\|RKMUB2 |
| | | ribulose-bisphosphate carboxylase (EC 4.1.1.39) |
| | | small chain B2 precursor - Arabidopsis |
| | | thaliana >gi\|16194\|emb\|CAA32701\| |
| | | (X14564) ribulose bisphosphate carboxylase |
| | | [Arabidopsis thaliana] Length = 181 |
| 678 | 2024678 | 1E-54 >gi\|3355475 (AC004218) |
| | | ribosomal protein L23a [Arabidopsis |
| | | thaliana] Length = 154 |
| 679 | 2024679 | Wd_Repeats(907-921) |
| 680 | 2024680 | 4E-31 >gb\|AAD15381\| (AC006223) |
| | | myosin II heavy chain [Arabidopsis |
| | | thaliana] Length = 1269 |
| 681 | 2024681 | Tyr_Phospho_Site(71-77) |
| 682 | 2024682 | 1E-128 >sp\|Q07098\|P2A1_ARATH SERINE/ |
| | | THREONINE PROTEIN |
| | | PHOSPHATASE PP2A-1 CATALYTIC |
| | | SUBUNIT >gi\|418779\|pir\|\|S31162 |
| | | phosphoprotein phosphatase (EC 3.1.3.16) 2A-alpha |
| | | catalytic chain (clone EP14a) - Arabidopsis |
| | | thaliana >gi\|166823 (M96733) protein |
| | | phosphatase [Arabidopsis |
| | | thaliana] >gi\|5091535\|gb\|AAD39564.1\|AC00706_4 |
| | | (AC007067) T10O24.4 [Arabidopsis thaliana] |
| | | Length = 306 |
| 683 | 2024683 | 1E-102 >gi\|2880054 (AC002340) |
| | | cytochrome P450 [Arabidopsis |
| | | thaliana] Length = 497 |
| 684 | 2024684 | 1E-55 >gi\|2213643 (U57338) |
| | | glossy1 homolog [Oryza sativa] |
| | | Length = 555 |
| 685 | 2024685 | 1E-103 >emb\|CAB45848.1\| (AL080254) |
| | | reticuline oxidase-like protein |
| | | [Arabidopsis thaliana] Length = 532 |
| 686 | 2024686 | 2E-74 ) >gb\|AAD32844.1\|AC007658_3 |
| | | (AC007658) thioredoxin-like protein |
| | | [Arabidopsis thaliana] Length = 130 |
| 687 | 2024687 | Pkc_Phospho_Site(223-225) |
| 688 | 2024688 | 3E-83 >emb\|CAA98170\| (Z73942) |
| | | RAB7C [Lotus japonicus] Length = 206 |
| 689 | 2024689 | 3'1E-100 >gi\|3122271\|sp\|O04294\|IMA2_ARATH IM- |
| | | PORTIN ALPHA-2 SUBUNIT |
| | | (KARYOPHERIN ALPHA-2 SUBUNIT) |
| | | (KAP ALPHA) >gi\|2154717\|emb\|CAA70703\| |
| | | (Y09511) Kap alpha protein [Arabidopsis thaliana] |
| | | Length = 531 |
| 690 | 2024690 | 2E-61 >gb\|AAD03449.1\| (AF118223) |
| | | contains similarity to Methanobacterium |
| | | thermoautotrophicum transcriptional regulator |
| | | (GB:AE000850) |
| | | [Arabidopsis thaliana] Length = 281 |
| 691 | 2024691 | 1E-124 >gi\|2829893 (AC002311) |
| | | phosphoglucomutase [Arabidopsis |
| | | thaliana] Length = 582 |
| 692 | 2024692 | 7E-83 >gi\|4206789 (AF112864) |
| | | syntaxin-related protein At-SYR1 |
| | | [Arabidopsis thaliana] Length = 346 |
| 693 | 2024693 | Tyr_Phospho_Site(1227-1235) |
| 694 | 2024694 | 8E-77 >emb\|CAB43837.1\| (AL078464) |
| | | proteinase-like protein [Arabidopsis |
| | | thaliana] Length = 816 |
| 695 | 2024695 | 7E-13 >emb\|CAA96548\| (Z72439) |
| | | major allergen Cor a 1 [Corylus |
| | | avellana] Length = 160 |
| 696 | 2024696 | Tyr_Phospho_Site(290-298) |
| 697 | 2024697 | 2E-48 >gb\|AAD18156\| (AC006260) |
| | | RNA-binding protein [Arabidopsis |
| | | thaliana] Length = 305 |
| 698 | 2024698 | 8E-36 >gi\|3850579 (AC005278) |
| | | Strong similarity to gb\|D14550 extracellular |
| | | dermal glycoprotein (EDGP) precursor from Daucus carota. |
| | | ESTs gb\|H37281, gb\|T44167, gb\|T21813, |
| | | gb\|N38437, gb\|Z26470, gb\|R65072, gb\|N76373, |
| | | gb\|F15470, gb\|Z35182, gb\|H76373, gb\|Z34678 |
| | | an... Length = 433 |
| 699 | 2024699 | 5E-45 >emb\|CAA69025\| (Y07745) |
| | | histone H2B like protein [Arabidopsis |
| | | thaliana] Length = 145 |
| 700 | 2024700 | Tyr_Phospho_Site(419-426) |
| 701 | 2024701 | Tyr_Phospho_Site(50-58) |
| 702 | 2024702 | 4E-63 >gb\|AAD27763.1\|AF077030_1 |
| | | (AF077030) hypothetical 43.2 kDa protein [Homo |
| | | sapiens] >gi\|4929577\|gb\|AAD34049.1\|AF151812_1 |
| | | (AF151812) CGI-54 |
| | | protein [Homo sapiens] Length 383 |
| 703 | 2024703 | 7E-64 >pir\|\|S71257 major latex protein type 1 - |
| | | Arabidopsis thaliana >gi\|1107493\|emb\|CAA63026\| |
| | | (X91960) major latex protein typel [Arabidopsis |
| | | thaliana] Length = 155 |
| 704 | 2024704 | 5E-14 >gi\|2252854 (AF013294) |
| | | similar to auxin-induced protein |
| | | [Arabidopsis thaliana] Length = 122 |
| 705 | 2024705 | 1E-126 >sp\|P11035\|N1A2_ARATH NITRATE |
| | | REDUCTASE 2 (NR2) >gi\|66202\|pir\|\|RDMUNH |
| | | nitrate reductase (NADH) (EC 1.6.6.1) 2 - Arabidopsis |
| | | thaliana >gi\|166782 (J03240) nitrate |
| | | reductase (EC 1.6.6.1) [Arabidopsis thaliana] |
| | | Length = 917 |
| 706 | 2024706 | 1E-79 >gb\|AAD18140\| (AC006260) 60S |
| | | ribosomal protein L12 |
| | | [Arabidopsis thaliana] Length = 166 |
| 707 | 2024707 | Tyr_Phospho_Site(1118-1125) |
| 708 | 2024708 | 4E-16 >gb\|AAD31580.1\|AC006922_12 |
| | | (AC006922) farnesylated protein |
| | | [Arabidopsis thaliana] Length = 329 |
| 709 | 2024709 | 1E-74 >sp\|P34788\|RS18_ARATH 40S |
| | | RIBOSOMAL PROTEIN |
| | | S18 >gi\|480908\|pir\|\|S37496 ribosomal |
| | | protein S18.A - Arabidopsis |
| | | thaliana >gi\|405613\|emb\|CAA80684\| |
| | | (Z23165) ribosomal protein S18A [Arabidopsis |
| | | thaliana] >gi\|434343\|emb\|CAA82273\| |
| | | (Z28701) S18 ribosomal protein [Arabidopsis |
| | | thaliana] >gi\|434345\|emb\|CAA82274\| |
| | | (Z28702) S18 ribosomal protein [Arabidopsis |
| | | thaliana] >gi\|434906\|emb\|CAA82275\| |
| | | (Z28962) S18 ribosomal protein [Arabidopsis |
| | | thaliana] >gi\|2505871\|emb\|CAA72909\| |
| | | (Y12227) ribosomal protein S18A [Arabidopsis |
| | | thaliana] >gi\|3287678 (AC003979) Match to |
| | | ribosomal S18 gene mRNA gb\|Z28701, DNA gb\|Z23165 |
| | | from A. thaliana. ESTs gb\|T21121, gb\|Z17755, |
| | | gb\|R64776 and gb\|R30430 come from this gene. |
| | | [Arabidopsis thaliana] >gi\|4538910\|emb\|CAB39647.1\| |
| | | (AL049482) S18.A ribosomal protein [Arabidopsis thaliana] |
| | | Length = 152 |
| 710 | 2024710 | Pkc_Phospho_Site(17-19) |
| 711 | 2024711 | 8E-83 >sp\|P24226\|HISX_BRAOC HISTIDINOL |
| | | DEHYDROGENASE, CHLOROPLAST PRECURSOR |
| | | (HDH) >gi\|99844\|pir\|\|A39358 histidinol |
| | | dehydrogenase (EC 1.1.1.23) precursor, chloroplast - |
| | | cabbage >gi\|167142 (M60466) histidinol dehydrogenase |
| | | [Brassica oleracea] Length = 469 |
| 712 | 2024712 | Tyr_Phospho_Site(42-50) |
| 713 | 2024713 | 4E-44 >emb\|CAB51195.1\| (AL096859) |
| | | glucuronosyl transferase-like protein |
| | | [Arabidopsis thaliana] Length = 385 |
| 714 | 2024714 | 1E-78 >emb\|CAA18477.1\| (AL022347) |
| | | serine/threonine kinase-like protein |
| | | [Arabidopsis thaliana] Length = 643 |
| 715 | 2024715 | 7E-38 >gb\|AAD17415\| (AC006248) |
| | | serine/threonine kinase [Arabidopsis |
| | | thaliana] Length = 365 |
| 716 | 2024716 | 9E-62 >emb\|CAB41340.1\| (AL049711) |
| | | dihydrolipoamide S-acetyltransferase precursor |
| | | [Arabidopsis thaliana] Length = 637 |
| 717 | 2024717 | Pkc_Phospho_Site(45-47) |
| 718 | 2024718 | Tyr_Phospho_Site(626-632) |
| 719 | 2024719 | Tyr_Phospho_Site(275-282) |
| 720 | 2024720 | 8E-38 >gb\|AAD51282.1\|AF159587_1 |
| | | (AF159587) far-red impaired response |
| | | protein [Arabidopsis thaliana] Length = 827 |
| 721 | 2024721 | Tyr_Phospho_Site(100-108) |
| 722 | 2024722 | Tyr_Phospho_Site(573-579) |
| 723 | 2024723 | 3E-79 >sp\|P25855\|GCSH_ARATH GLYCINE |
| | | CLEAVAGE SYSTEM H |
| | | PROTEIN PRECURSOR >gi\|166725 (M82921) |
| | | H-Protein precursor [Arabidopsis |
| | | thaliana] >gi\|861215 (U27144) |
| | | glycine decarboxylase complex H-protein precursor |
| | | [Arabidopsis thaliana] >gi\|3608151 |
| | | (AC005314) glycine decarboxylase complex H- |
| | | protein [Arabidopsis thali- |
| | | ana] >gi\|445119\|prf\|\|1908425A |
| | | Gly decarboxylase:SUBUNITH protein |
| | | [Arabidopsis thaliana] Length = 165 |
| 724 | 2024724 | Pkc_Phospho_Site(12-14) |
| 725 | 2024725 | Tyr_Phospho_Site(7-15) |
| 726 | 2024726 | 1E-37 >gi\|2160133 (AC000375) |
| | | Strong similarity to Arabidopsis |
| | | gb\|X91953,F19K23.3,F19K23.15. ESTs |
| | | gb\|T21984,gb\|ATTSQ219,gb\|ATTS0207,gb\|T21984 |
| | | come from this gene. |
| | | [Arabidopsis thaliana] Length = 150 |
| 727 | 2024727 | 1E-11 >sp\|Q00808\|HET1_PODAN VEGE- |
| | | TATIBLE INCOMPATIBILITY |
| | | PROTEIN HET-E-1 >gi\|607003 (L28125) |
| | | beta transducin-Iike protein [Podospora |
| | | anserina] Length = 1356 |
| 728 | 2024728 | Tyr_Phospho_Site(1 233-1240) |
| 729 | 2024729 | Tyr_Phospho_Site(646-654) |
| 730 | 2024730 | 3E-68 >sp\|P36212\|R12C_ARATH 50S |
| | | RIBOSOMAL PROTEIN L12-C, |
| | | CHLOROPLAST PRECURSOR |
| | | (CL12-C) >gi\|541897\|pir\|\|C53394 ribosomal |
| | | protein L12.C, chloroplast - Arabidopsis |
| | | thaliana >gi\|468773\|emb\|CAA48183\| |
| | | (X68046) ribosomal protein L12 |
| | | [Arabidopsis thaliana] Length = 187 |
| 731 | 2024731 | 9E-40 >emb\|CAA16752.1\| (AL021711) |
| | | protein kinase-like protein |
| | | [Arabidopsis thaliana] Length 421 |
| 732 | 2024732 | Pkc_Phospho_Site(67-69) |
| 733 | 2024733 | 1E-67 >sp\|P36428\|SYA_ARATH ALANYL- |
| | | TRNA SYNTHETASE, |
| | | MITOCHONDRIAL PRECURSOR |
| | | (ALANINE-TRNA LIGASE) |
| | | (ALARS) >gi\|1673365\|emb\|CAA80380\| |
| | | (Z22673) mitochondrial tRNA-Ala synthetase |
| | | [Arabidopsis thaliana] Length = 1003 |
| 734 | 2024734 | Tyr_Phospho_Site(124-130) |
| 735 | 2024735 | Pkc_Phospho_Site(23-25) |
| 736 | 2024736 | Tyr_Phospho_Site(194-201) |
| 737 | 2024737 | 2E-57 >sp\|P51419\|RL27_ARATH 60S |
| | | RIBOSOMAL PROTEIN |
| | | L27 >gi\|2244857\|emb\|CAB10279.1\| |
| | | (Z97337) ribosomal protein [Arabidopsis thaliana] |
| | | Length = 135 |
| 738 | 2024738 | Tyr_Phospho_Site(958-965) |
| 739 | 2024739 | 6E-11 >gi\|3341687 (AC003672) |
| | | ras protein [Arabidopsis thaliana] |
| | | Length = 93 |
| 740 | 2024740 | 0 >prf\|\|1804333D Gln synthetase |
| | | [Arabidopsis thaliana] Length = 430 |
| 741 | 2024741 | 5E-84 >dbj\|BAA85109.1\| (AB030732) |
| | | Cys2/His2-type zinc finger protein 3 |
| | | [Arabidopsis thaliana] Length = 193 |
| 742 | 2024742 | 1E-12 >emb\|CAB43438.1\| (AL050300) |
| | | protein [Arabidopsis thaliana] |
| | | Length = 541 |
| 743 | 2024743 | 2E-36 >gb\|AAD31078.1\|AC007357_27 |
| | | (AC007357) Contains PF100097 Zinc |
| | | finger (C3HC4) ring finger motif. [Arabidopsis |
| | | thaliana] Length = 260 |
| 744 | 2024744 | Rgd(902-904) |
| 745 | 2024745 | Tyr_Phospho_Site(472-478) |
| 746 | 2024746 | 6E-52 >gb\|AAF00626.1\|AC009540_3 |
| | | (AC009540) GAR1 protein [Arabidopsis |
| | | thaliana] >gi\|6223652\|gb\|AAF05866.1\|AC011698_17 |
| | | (AC011698) unknown |
| | | protein [Arabidopsis thaliana] Length 219 |
| 747 | 2024747 | Tyr_Phospho_Site(27-34) |
| 748 | 2024748 | 3E-75 >sp\|P08927\|RUBB_PEA RUBISCO |
| | | SUBUNIT BINDING-PROTEIN |
| | | BETA SUBUNIT PRECURSOR (60 KD |
| | | CHAPERONIN BETA SUBUNIT) (CPN-60 |
| | | BETA) >gi\|806808 (U21139) chaperonin precursor |
| | | [Pisum sativum] Length = 595 |
| 749 | 2024749 | 9E-72 >pir\|\|D36571 ubiquitin |
| | | 81-aa extension protein 2 - Arabidopsis |
| | | thaliana >gi\|166936 (J05540) ubiquitin |
| | | extension protein (UBQ6) [Arabidopsis |
| | | thaliana] >gi\|3522953\|gb\|AAC34235.1\| |
| | | (AC004411) ubiquitin extension protein |
| | | (UBQ6) [Arabidopsis thaliana] Length = 157 |
| 750 | 2024750 | 1E-129 >sp\|Q40082\|XYLA_HORVU XYLOSE |
| | | ISOMERASE >gi\|2130052\|pir\|\|S65467 xylose |
| | | isomerase (EC 5.3.1.5) - |
| | | barley >gi\|1296809\|emb\|CAA64545\| (X95257) |
| | | xylose isomerase [Hordeum vulgare] |
| | | Length = 479 |
| 751 | 2024751 | 9E-57 >sp\|P27202\|PSBR_ARATH PHOTO- |
| | | SYSTEM 1110 KD POLYPEPTIDE |
| | | PRECURSOR >gi\|72714\|pir\|\|F2MU10 photosystem |
| | | II 10K protein precursor - Arabidopsis |
| | | thaliana >gi\|16447\|emb\|CAA39441\| |
| | | (X55970) photosystem II 10 kDa polypeptide [Arabidopsis |
| | | thaliana] >gi\|3152571 (AC002986) Match |
| | | to photosystem II 10kDa polypeptide gb\|X55970. |
| | | ESTs gb\|Z17693, gb\|N37616, gb\|T41858, |
| | | gb\|T88021, gb\|R37531, gb\|T04679, gb\|N37520, |
| | | gb\|N64965, gb\|Z17592 and gb\|N65338, |
| | | gb\|N37466 and gb\|T45400 come from this gene. |
| | | [Arabidopsis ... Length = 140 |
| 752 | 2024752 | 2E-11 >gb\|AAD48957.1\|AF149414_6 |
| | | (AF149414) contains similarity to Pfam |
| | | family PF00646 (F-box domain); score=11/3, E=0.23, |
| | | N=1 [Arabidopsis thaliana] |
| | | Length = 378 |
| 753 | 2024753 | 1E-120 >gi\|3927825 (AC005727) |
| | | dTDP-glucose 4-6-dehydratase |
| | | [Arabidopsis thaliana] Length = 343 |
| 754 | 2024754 | 3E-43 >gi\|2708747 (AC003952) |
| | | glycine-rich, zinc-finger DNA-binding |
| | | protein [Arabidopsis thaliana] Length = 299 |
| 755 | 2024755 | 1E-50 >pdb\|\|E2C\|A Chain A, |
| | | E2-C, An Ubiquitin Conjugating Enzyme |
| | | Required For The Destruction Of Mitotic |
| | | Cyclins >gi\|3660188\|pdb\|\|E2C\|B Chain |
| | | B, E2-C, An Ubiquitin Conjugating Enzyme Required For |
| | | The Destruction Of Mitotic |
| | | Cyclins >gi\|3660189\|pdb\|\|E2C\|C Chain C, |
| | | E2-C, |
| 756 | 2024756 | Pkc_Phospho_Site(27-29) |
| 757 | 2024757 | 3E-20 >sp\|P47198\|RL22_RAT 60S |
| | | RIBOSOMAL PROTEIN |
| | | L22 >gi\|1083790\|pir\|\|S52084 ribosomal |
| | | protein L22 - rat >gi\|710295\|emb\|CAA55204\| |
| | | (X78444) ribosomal protein L22 [Rattus |
| | | norvegicus] >gi\|1093952\|prf\|\|2105193A |
| | | ribosomal protein |
| 758 | 2024758 | Tyr_Phospho_Site(999-1005) |
| 759 | 2024759 | 7E-91 >sp\|Q43291\|RL2I_ARATH 60S |
| | | RIBOSOMAL PROTEIN |
| | | L21 >gi\|2160162 (AC000132) Similar to ribosomal |
| | | protein L21 (gb\|L38826). ESTs |
| | | gb\|AA395597,gb\|ATTS5197 come from this gene. |
| | | [Arabidopsis thaliana] >gi\|3482935 |
| | | (AC003970) ribosomal protein L21 |
| | | [Arabidopsis thaliana] Length = 164 |
| 760 | 2024760 | 2E-89 >gi\|2443883 (AC002294) |
| | | Similar to RPS-2 disease resistance |
| | | protein [Arabidopsis thaliana] Length = 967 |
| 761 | 2024761 | 2E-18 >gb\|AAD31580.1\|AC006922_12 |
| | | (AC006922) farnesylated protein |
| | | [Arabidopsis thaliana] Length = 329 |
| 762 | 2024762 | Tyr_Phospho_Site(166-173) |
| 763 | 2024763 | 1E-1 1 >emb\|CAA85467.1\| (Z37093) |
| | | weak similarity with gamma-interferon |
| | | inducible protein IP-30 (Swiss Prot accession number |
| | | P13284) [Caenorhabditis elegans] Length = 264 |
| 764 | 2024764 | Pkc_Phospho_Site(67-69) |
| 765 | 2024765 | 4E-60 >gb\|AAD50027.1\|AC00765_22 |
| | | (AC007651) Similar to leucine-rich receptor-like protein |
| | | kinase [Arabidopsis thaliana] Length = 1133 |
| 766 | 2024766 | 1E-17 >gi\|4206767 (AF104330) |
| | | glycine-rich protein 3 short isoform |
| | | [Arabidopsis thaliana] Length = 116 |
| 767 | 2024767 | Pkc_Phospho_Site(7-9) |
| 768 | 2024768 | 3E-74 >dbj\|BAA36335\| (AB015141) |
| | | AHP1 [Arabidopsis |
| | | thaliana] >gi\|4156245\|dbj\|BAA37112\| |
| | | (AB012570) ATHP3 [Arabidopsis thaliana] |
| | | Length = 154 |
| 769 | 2024769 | 7E-61 >gi\|3894193 (AC005662) |
| | | strictosidine synthase [Arabidopsis |
| | | thaliana] Length = 395 |
| 770 | 2024770 | 4E-94 ) >sp\|P46283\|S17P_ARATH SEDO- |
| | | HEPTULOSE-1,7-BISPHOSPHATASE, |
| | | CHLOROPLAST PRECURSOR (SEDOHEPTULOSE- |
| | | BISPHOSPHATASE) (SBPASE) |
| | | (SED(1,7)P2ASE) >gi\|1076403\|pir\|S51838 |
| | | sedoheptulose-1,7-biphosphatase - Arabidopsis |
| | | thaliana >gi\|786466\|bbs\|159034 |
| | | (S74719) sedoheptulose-1,7-bisphosphatase, SBPase |
| | | {EC 3.1.3.37}[Arabidopsis thaliana, |
| | | C24, Peptide Chloroplast, 393 aa] |
| | | [Arabidopsis thaliana] Length = 393 |
| 771 | 2024771 | Tyr_Phospho_Site(325-333) |
| 772 | 2024772 | 6E-67 >sp\|Q07511\|FDH_SOLTU MITO- |
| | | CHONDRIAL FORMATE |
| | | DEHYDROGENASE PRECURSOR |
| | | (NAD-DEPENDENT FORMATE |
| | | DEHYDROGENASE) |
| | | (FDH) >gi\|542089\|pir\|\|JQ2272 formate |
| | | dehydrogenase (EC 1.2.1.2) precursor, mitochondrial - |
| | | potato >gi\|297798\|emb\|CAA79702\| (Z21493) |
| | | mitochondrial formate dehydrogenase precursor |
| | | [Solanum tuberosum] Length = 379 |
| 773 | 2024773 | 3E-41 >emb\|CAA66408\| (X97829) |
| | | product similar to ccr protein, Citrus |
| | | paradisi; PIR: S52663 [Arabidopsis |
| | | thaliana] >gi\|1550735\|emb\|CAA66824\| |
| | | (X98130) unknown [Arabidopsis thaliana] |
| | | Length = 141 |
| 774 | 2024774 | Tyr_Phospho_Site(1099-1106) |
| 775 | 2024775 | Tyr_Phospho_Site(853-859) |
| 776 | 2024776 | Tyr_Phospho_Site(962-969) |
| 777 | 2024777 | 2E-66 >gb\|AAD22351.1\|AC006592_8 |
| | | (AC006592) mitochondrial uncoupling |
| | | protein [Arabidopsis thaliana] Length = 313 |
| 778 | 2024778 | 1E-104 >gi\|2829923 (AC002291) |
| | | Similar to uridylyl transferases |
| | | [Arabidopsis thaliana] Length = 453 |
| 779 | 2024779 | 4E-68 >gi\|2218152 (AF005279) |
| | | type lila membrane protein cp-wap13 |
| | | [Vigna unguiculata] Length = 346 |
| 780 | 2024780 | Rgd(490-492) |
| 781 | 2024781 | Pkc_Phospho_Site(57-59) |
| 782 | 2024782 | 5E-20 >emb\|CAA19701.1\| (AL024486) |
| | | lectin like protein [Arabidopsis |
| | | thaliana] Length = 246 |
| 783 | 2024783 | 5E-56 >sp\|P55871\|IF2B_MALDO EUKAR- |
| | | YOTIC TRANSLATION INITIATION |
| | | FACTOR 2 BETA SUBUNIT |
| | | (EIF-2-BETA) >gi\|1732361 (U80269) translation |
| | | initiation factor 2 beta [Malus domestica] |
| | | Length = 307 |
| 784 | 2024784 | Pkc_Phospho_Site(23-25) |
| 785 | 2024785 | 9E-32 >sp\|P46600\|HAT1_ARATH HOMEO- |
| | | BOX-LEUCINE ZIPPER PROTEIN |
| | | HAT1 (HD-ZIP PROTEIN 1) >gi\|549883 |
| | | (U09332) homeobox protein [Arabidopsis |
| | | thaliana] >gi\|549884 (U09333) |
| | | homeobox protein [Arabidopsis |
| | | thaliana] >gi\|2245105\|emb\|CAB10S27.1\| |
| | | (Z97343) homeobox-leucine zipper protein HAT1 |
| | | (hd-zip protein 1) [Arabidopsis thaliana] |
| | | Length = 282 |
| 786 | 2024786 | 1E-44 >dbj\|BAA819l0.1\| (AB011262) |
| | | nuclear transport factor 2 (NTF2) |
| | | [Oryza sativa] Length = 122 |
| 787 | 2024787 | 1E-10 >emb\|CAA18838.1\| (AL023094) |
| | | bZIP transcription factor ATB2 |
| | | [Arabidopsis thaliana] Length 159 |
| 788 | 2024788 | 1E-1 24 >gi\|3980393 (AC004561) |
| | | glutathione 5-transferase [Arabidopsis |
| | | thaliana] Length = 227 |
| 789 | 2024789 | 1E-116 >emb\|CAA11414\| (AJ223496) |
| | | phosphoenolpyrovate carboxylase |
| | | [Brassica juncea] Length = 964 |
| 790 | 2024790 | 1E-110 >emb\|CAB52677.1\| (AJ245907) |
| | | photosystem I subunit II precursor |
| | | [Arabidopsis thaliana] Length = 204 |
| 791 | 2024791 | Tyr_Phospho_Site(1255-1262) |
| 792 | 2024792 | Pkc_Phospho_Site(166-168) |
| 793 | 2024793 | 6E-65 >dbj\|BAA19529\| (AB002560) |
| | | CUC2 [Arabidopsis thaliana] Length = 375 |
| 794 | 2024794 | Pkc_Phospho_Site(40-42) |
| 795 | 2024795 | 6E-63 >emb\|CAA66909\| (X98255) |
| | | transcriptionally stimulated by |
| | | gibberellins; expressed in meristematic region, and |
| | | style [Arabidopsis thaliana] Length = 106 |
| 796 | 2024796 | 1E-129 >emb\|CAB39626.1\| (AL049481) |
| | | oxidoreductase [Arabidopsis |
| | | thaliana] Length = 389 |
| 797 | 2024797 | 1E-101 >sp\|Q38937\|RACS_ARATH RAC- |
| | | LIKE GTP BINDING PROTEIN |
| | | ARAC5 >gi\|1293668 (U52350) GTP-binding protein |
| | | [Arabidopsis thaliana] Length = 196 |
| 798 | 2024798 | Tyr_Phospho_Site(505-512) |
| 799 | 2024799 | 2E-61 >gi\|2252850 10 (AF013294) |
| | | contains region of similarity to DNA |
| | | binding protein [Arabidopsis thaliana] |
| | | Length = 575 |
| 800 | 2024800 | 1E-85 >sp\|P21238\|RUBA_ARATH RUBISCO |
| | | SUBUNIT BINDING-PROTEIN |
| | | ALPHA SUBUNIT PRECURSOR (60 |
| | | KD CHAPERONIN ALPHA SUBUNIT) |
| | | (CPN-60 ALPHA) >gi\|2129561\|pir\|\|S71235 |
| | | chaperonin-60 alpha chain - Arabidopsis |
| | | thaliana >gi\|1223910 (U49357) |
| | | chaperonin-60 alpha subunit [Arabidopsis |
| | | thaliana] >gi\|4510416\|gb\|AAD21502.1\| |
| | | (AC006929) rubisco binding protein alpha subunit |
| | | [Arabidopsis thaliana] Length = 586 |
| 801 | 2024801 | Tyr_Phospho_Site(662-669) |
| 802 | 2024802 | Pkc_Phospho_Site(45-47) |
| 803 | 2024803 | 3E-15 >dbj\|BAA77204.1\| (AB026262) |
| | | ring finger protein [Cicer arietinum] |
| | | Length = 131 |
| 804 | 2024804 | Tyr_Phospho_Site(517-523) |
| 805 | 2024805 | 4E-17 >emb\|CAA04730\| (AJ001401) |
| | | HpnA protein [Zymomonas mobilis] |
| | | Length = 337 |
| 806 | 2024806 | 1E-23 >ref\|NP_002803.1\|PPSMD8\| |
| | | proteasome (prosome, macropain) 26S subunit, non-ATPase, |
| | | 8 >gi\|1346766\|sp\|P48556\|PSD8 HUMAN 26S |
| | | PROTEASOME REGULATORY SUBUN IT S14 |
| | | (P31) >gi\|136274\|pir\|\|S56108 |
| | | multicatalytic endopeptidase complex (EC 3.4.99.46) |
| | | regulatory chain 31 - |
| | | human >gi\|1037164\|dbj\|BAA07237\| |
| | | (D38047) 26S proteasome subunit p31 [Homo |
| | | sapiens] >gi\|3702282 (AC005789) PP31_HUMAN |
| | | [Homo sapiens] Length = 257 |
| 807 | 2024807 | Tyr_Phospho_Site(162-169) |
| 808 | 2024808 | 1E-50 >gb\|AAD153451\| (AC004044) |
| | | small nuclear riboprotein Sm-D1 |
| | | [Arabidopsis thaliana] Length = 116 |
| 809 | 2024809 | Pkc_Phospho_Site(61-63) |
| 810 | 2024810 | Rgd(360-362) |
| 811 | 2024811 | Tyr_Phospho_Site(528-535) |
| 812 | 2024812 | 5E-20 >emb\|CAA77232\| (Y18620) |
| | | DsPTP1 protein [Arabidopsis thaliana] |
| | | Length = 198 |
| 813 | 2024813 | 8E-55 ) >emb\|CAA11524.1\| (AJ223634) |
| | | transcription factor IIA small subunit [Arabidopsis |
| | | thaliana] >gi\|5051786 \|emb\|CAB45079.1\| |
| | | (AL078637) transcription factor IIA small subunit |
| | | [Arabidopsis thaliana] Length = 106 |
| 814 | 2024814 | 2E-37 >gi\|2651314 (AC002336) |
| | | ribosomal protein S26 [Arabidopsis |
| | | thaliana] Length = 133 |
| 815 | 2024815 | 5E-76 >emb\|CAB36783.1\| (AL035525) |
| | | aminopeptidase-like protein [Arabidopsis thaliana] |
| | | Length = 873 |
| 816 | 2024816 | 1E-75 >sp\|P49203\|RS13_ARATH 40S |
| | | RIBOSOMAL PROTEIN S13 Length = 150 |
| 817 | 2024817 | 1E-111 >sp\|Q01908\|ATP1_ARATH ATP |
| | | SYNTHASE GAMMA CHAIN 1, CHLOROPLAST |
| | | PRECURSOR >gi\|81635\|pir\|\|B39732 H+-transporting |
| | | ATP synthase (EC 3.6.1.34) gamma-I chain precursor, |
| | | chloroplast - Arabidopsis thaliana >gi\|166632 |
| | | (M61741) ATP synthase gamma-subunit [Arabidopsis |
| | | thaliana] >gi\|5732056\|gb\|AAD48955.1\|AF149414_4 |
| | | (AF149414) Arabidopsis thaliana APCI-ATP synthase |
| | | gamma chain 1 (GB:M61741); contains similarity to |
| | | Pfam PF00231 -ATP synthase; score=658.6, |
| | | E=3.1e-194n n+1 Length = 373 |
| 818 | 2024818 | Tyr_Phospho_Site(58-64) |
| 819 | 2024819 | 2E-26 >gi\|3236253 (AC004684) |
| | | receptor-like protein kinase [Arabidopsis |
| | | thaliana] Length = 675 |
| 820 | 2024820 | Rgd(217-219) |
| 821 | 2024821 | 2E-85 ) >sp\|P35131\|UBC8_ARATH UBIQ- |
| | | UITIN-CONJUGATING ENZYME E2- |
| | | 17 KD 8 (UBIQUITIN-PROTEIN LIGASE 8) |
| | | (UBIQUITIN CARRIER PROTEIN 8) |
| | | (UBCAT4A) >gi\|398699\|emb\|CAA78713\| |
| | | (Z14989) ubiquitin conjugating enzyme |
| | | homolog [Arabidosis thaliana] Length = 148 |
| 822 | 2024822 | Tyr_Phospho_Site(473-480) |
| 823 | 2024823 | Tyr_Phospho_Site(227-235) |
| 824 | 2024824 | Pkc_Phospho_Site(54-56) |
| 825 | 2024825 | Serpin(547-557) |
| 826 | 2024826 | 1E-105 >emb\|CAA16700.1\| (AL021687) |
| | | kinase-like protein [Arabidopsis |
| | | thaliana] Length 290 |
| 827 | 2024827 | 2E-57 >gi\|3859606 (AF104919) |
| | | contains similarity to cysteine proteases |
| | | (Pfam: PF00112, E=1.3e-79, N=1) |
| | | [Arabidopsis thaliana] Length = 359 |
| 828 | 2024828 | 3E-70 >gi\|3980378 (AC004561) |
| | | RNA binding protein [Arabidopsis |
| | | thaliana] Length = 483 |
| 829 | 2024829 | 7E-60 >gi\|3687249 (AC005169) |
| | | copia-like transposable element |
| | | [Arabidopsis thaliana] Length = 122 |
| 830 | 2024830 | 4E-37 >emb\|CAB36546.1\| (AL035440) |
| | | DNA binding protein [Arabidopsis |
| | | thaliana] Length = 427 |
| 831 | 2024831 | 0 >gbjAAD41432.1\|AC007727_21 (AC007727) |
| | | Contains similarity to gb\|AJ000644 SPOP |
| | | (speckle-type POZ protein) from Homo sapiens and |
| | | contains a PF\|00651 BTB/POZ domain. ESTs |
| | | gb\|T75841, gb\|R89974, gb\|R30221, |
| | | gb\|N96386, gb\|T76457, gb\|A1100013 and gb... |
| | | Length = 326 |
| 832 | 2024832 | 1E-67 >gb\|AAD20138\| (AC006282) |
| | | 60S ribosomal protein L24 [Arabidopsis |
| | | thaliana] >gi\|4581159\|gb\|AAD24643.1\|AC006919_21 |
| | | (AC006919) 60S ribosomal protein L24 |
| | | [Arabidopsis thaliana] Length = 177 |
| 833 | 2024833 | 2E-80 >sp\|Q42340\|RS16_ARATH 40S |
| | | RIBOSOMAL PROTEIN S16 Length = 146 |
| 834 | 2024834 | 1E-126 ) >sp\|Q06402\|1A12_ARATH 1- |
| | | AMINOCYCLOPROPANE-2-CARBOXYLATE |
| | | SYNTHASE 2 (ACC SYNTHASE 2) |
| | | (S-ADENOSYL-L-METHIONINE |
| | | METHYLTHIOADENOSINE-LYASE |
| | | 2) >gi\|476924\|pir\|\|A47199 1- |
| | | aminocyclopropane-1-carboxylate synthase |
| | | (EC 4.4.1.14) - |
| 835 | 2024835 | 7E-25 >gi\|2264378 (AC002354) |
| | | bZIP-Iike transcription factor |
| | | [Arabidopsis thaliana] Length = 669 |
| 836 | 2024836 | 6E-49 >gi\|3603473 (AF090698) |
| | | elicitor-responsive gene-3 [Oryza sativa] |
| | | Length = 144 |
| 837 | 2024837 | Tyr_Phospho_Site(467-473) |
| 838 | 2024838 | 2E-98 >gb\|AAD33097.1\|AF082525_1 |
| | | (AF082525) homoserine kinase |
| | | [Arabidopsis thaliana] Length = 370 |
| 839 | 2024839 | Tyr_Phospho_Site(494-501) |
| 840 | 2024840 | 4E-57 >gi\|1063415 (L40948) |
| | | K+ channel protein [Arabidopsis thaliana] |
| | | Length = 328 |
| 841 | 2024841 | 8E-66 >gi\|3176663 (AC004393) |
| | | Contains similarity to S-receptor kinase |
| | | 8 precursor gb\|D38563 from Brassica rapa. |
| | | ESTs gb\|T88253 and gb\|AA394649 come from this gene. |
| | | [Arabidopsis thaliana] Length = 389 |
| 842 | 2024842 | 3E-37 >emb\|CAB37534\| (AL035538) |
| | | MADS-box protein AGL17-like |
| | | protein [Arabidopsis thaliana] Length 228 |
| 843 | 2024843 | Tyr_Phospho_Site(179-185) |
| 844 | 2024844 | 1E-85 >gi\|2583124 (AC002387) |
| | | 5-enolpyruvylshikimate-3-phosphate synthase |
| | | (EPSP) [Arabidopsis thaliana] Length = 520 |
| 845 | 2024845 | 3E-51 >gi\|3355468 (AC004218) |
| | | ribosomal protein L35 [Arabidopsis |
| | | thaliana] Length = 123 |
| 846 | 2024846 | 1E-64 ) >emb\|CAB51209.1\| (AL096860) |
| | | 40S RIBOSOMAL PROTEIN S20 |
| | | homolog [Arabidopsis thaliana] Length = 122 |
| 847 | 2024847 | 3E-72 >gi\|4093155 (AF088281) |
| | | phytochrome-associated protein I |
| | | [Arabidopsis thaliana] Length = 267 |
| 848 | 2024848 | Tyr_Phospho_Site(621-627) |
| 849 | 2024849 | 4E-86 >emb\|CAB10353.1\| (Z97339) |
| | | hypothetical protein [Arabidopsis |
| | | thaliana] >gi\|3426058\|emb\|CAA07072.1\| |
| | | (AJ007585) IB1P8-4 protein [Arabidopsis |
| | | thaliana] Length = 171 |
| 850 | 2024850 | 1E-25 >sp\|Q06548\|APKA_ARATH PROTEIN |
| | | KINASE APK1A >gi\|282877\|pir\|\|S28615 |
| | | protein kinase, tyrosine/serine/threonine-specific (EC |
| | | 2.7.1 .-) - Arabidopsis |
| | | thaliana >gi\|217829\|dbj\|BAA02092\| |
| | | (D12522) protein tyrosine-serine-threonine |
| 851 | 2024851 | 7E-23 >sp\|P55610\|Y4PA_RHISN TRAN- |
| | | SCRIPTIONAL REGULATORY PROTEIN |
| | | Y4PA >gi\|2182569 (AE000089) Y4pA |
| | | [Rhizobium sp. NGR234] Length = 609 |
| 852 | 2024852 | 7E-51 >emb\|CAB41143.1 (AL049658) |
| | | peptide transporter [Arabidopsis |
| | | thaliana] Length = 450 |
| 853 | 2024853 | 2E-33 >gi\|2984225 (AE000766) |
| | | enolase-phosphatase E-1 [Aquifex |
| | | aeolicus] Length = 223 |
| 854 | 2024854 | 4E-77 >gi\|2191149 (AF007269) |
| | | Similar to protein kinase [Arabidopsis |
| | | thaliana] Length = 450 |
| 855 | 2024855 | 3E-71 ) >pir\|\|S58123 thioredoxin - |
| | | Arabidopsis thaliana >gi\|992964\|emb\|CAA84612\| |
| | | (Z35475) thioredoxin [Arabidopsis thaliana] |
| | | Length = 133 |
| 856 | 2024856 | Tyr_Phospho_Site 565-572 |
| 857 | 2024857 | Rgd(833-835) |
| 858 | 2024858 | 3E-83 ) >gb\|AAD41076.1\|AF141202_1 |
| | | (AF141202) EIN2 [Arabidopsis |
| | | thaliana] >gi\|5231115\|gb\|AAD41077.1\|AF141203_1 |
| | | (AF141203) EIN2 [Arabidopsis |
| | | thaliana] Length = 1294 |
| 859 | 2024859 | Tyr_Phospho_Site(57-64) |
| 860 | 2024860 | Tyr_Phospho_Site(598-604) |
| 861 | 2024861 | 3E-15 >gi\|3176673 (AC003671) |
| | | Similar to serine/threonine kinase |
| | | gb\|Y12531 from Brassica oleracea. |
| | | [Arabidopsis thaliana] Length = 321 |
| 862 | 2024862 | 5'1E-31 >gi\|5903082\|gb\|AAD55640.1\|AC008017_13 |
| | | (AC008017) Similar to downy mildew resistance protein |
| | | RPP5 [Arabidopsis thaliana] Length = 176 |
| 863 | 2024863 | Tyr_Phospho_Site(1029-1036) |
| 864 | 2024864 | 3E-29 >gb\|AAD14532\| (AC006200) |
| | | membrane transporter [Arabidopsis |
| | | thaliana] Length = 725 |
| 865 | 2024865 | Pkc PhosphoSite(36-38) |
| 866 | 2024866 | Tyr PhosphoSite(177-185) |
| 867 | 2024867 | 9E-89 >sp\|P50318\|PGKH_ARATH PHOS- |
| | | PHOGLYCERATE KINASE, |
| | | CHLOROPLAST >gi\|2129669\|pir\|\|S71368 |
| | | phosphoglycerate kinase - Arabidopsis |
| | | thaliana (fragment) >gi\|1022805\|gb\|AAB60303.1\| |
| | | (U37701) phosphoglycerate |
| | | kinase [Arabidopsis thaliana] Length = 399 |
| 868 | 2024868 | Tyr_Phospho_Site(1181-1187) |
| 869 | 2024869 | 5'Rgd(763-765) |
| 870 | 2024870 | 5E-83 >gi\|2459417 (AC002332) |
| | | pre-mRNA splicing factor PRP19 |
| | | [Arabidopsis thaliana] Length = 540 |
| 871 | 2024871 | Tyr_Phospho_Site(312-320) |
| 872 | 2024872 | Pkc_Phospho_Site(26-28) |
| 873 | 2024873 | 2E-92 >sp\|P29510\|TBA2_ARATH TUBULIN |
| | | ALPHA-2/ALPHA-4 CHAIN >gi\|320183\|pir\|\|JQ1594 |
| | | tubulin alpha chain - Arabidopsis thaliana >gi\|166914 |
| | | (M84696) apha-2 tubulin [Arabidopsis |
| | | thaliana] >gi\|166916 (M84697) alpha-4 |
| | | tubulin [Arabidopsis thaliana] Length = 450 |
| 874 | 2024874 | Tyr_Phospho_Site(1025-1032) |
| 875 | 2024875 | Rgd(354-356) |
| 876 | 2024876 | Tyr_Phospho_Site(239-247) |
| 877 | 2024877 | 4E-13 >sp\|O14069\|YEA4_SCHPO PROBABLE |
| | | 60s RIBOSOMAL PROTEIN |
| | | C2E11.04 >gi\|3395568\|emb\|CAA20151\| |
| | | (AL031181) 60s ribosomal protein L28 |
| | | [Schizosaccharomyces |
| | | pombe] >gi\|4106660\|emb\|CAA22600\| |
| | | (AL035064) 60s ribosomal protein 128 |
| | | [Schizosacoharomyces pombe] Length = 134 |
| 878 | 2024878 | 3E-74 >emb\|CAA18853.1\| (AL023094) |
| | | amidophosphoribosyltransferase 2 |
| | | precursor (Arabidopsis thaliana] Length = 561 |
| 879 | 2024879 | 1E-109 >sp\|P45432\|FUS6_ARATH FUSCA |
| | | PROTEIN FUS6 >gi\|432446 (L26498) |
| | | FUS6 [Arabidopsis thaliana] Length = 441 |
| 880 | 2024880 | Pkc_Phospho_Site(52-54) |
| 881 | 2024881 | 3E-59 >gi\|2262176 (AC002329) |
| | | RING zinc-finger protein [Arabidopsis |
| | | thaliana] >gi\|3790573 (AF078824) |
| | | RING-H2 finger protein RHA3a [Arabidopsis |
| | | thaliana] >gi\|4914367\|gb\|AAD32903.1\|AC007584_1 |
| | | (AC007584) zinc finger |
| | | protein [Arabidopsis thaliana] Length = |
| 882 | 2024882 | 3E-48 >gi\|473878 (U08315) |
| | | calnexin homolog [Arabidopsis thaliana] |
| | | Length = 528 |
| 883 | 2024883 | 7E-16 >dbj\|BAA81662.2\| (AB029060) |
| | | F1F0-ATPase inhibitor protein [Oryza |
| | | sativa] Length = 123 |
| 884 | 2024884 | 3E-36 >gb\|AAD34430.1\|AF084446_1 |
| | | (AF084446) calmodulin mutant SYNCAM36 |
| | | [synthetic construct] Length = 149 |
| 885 | 2024885 | Tyr_Phospho_Site(4-11) |
| 886 | 2024886 | 1E-59 >gi\|3157937 (AC002131) |
| | | Identical to aspartic proteinase cDNA |
| | | gb\|U51036 from A. thaliana. ESTs gb\|N96313, |
| | | gb\|T21893, gb\|R30158, gb\|T21482, gb\|T43650, |
| | | gb\|R64749, gb\|R65157, gb\|T88269, gb\|T44552, |
| | | gb\|T22542, gb\|T76533, gb\|T44350, gb\|Z34591, |
| | | gb\|AA728734, gb... Length = 506 |
| 887 | 2024887 | 3E-58 >sp\|Q39411\|RL26_BRARA 60S |
| | | RIBOSOMAL PROTEIN |
| | | L26 >gi\|2160300\|dbj\|BAA18941\| (078495) |
| | | ribosomal protein [Brassica rapa] |
| | | Length = 146 |
| 888 | 2024888 | 9E-73 >sp\|P43297\|RD21_ARATH CYSTEINE |
| | | PROTEINASE RD21A |
| | | PRECURSOR >gi\|541857\|pir\|\|JN0719 |
| | | drought-inducible cysteine proteinase (EC |
| | | 3.4.22.-) RD21A precursor - Arabidopsis |
| | | thaliana >gi\|435619\|dbj\|BAA02374\| |
| | | (D13043) thiol protease [Arabidopsis thaliana] |
| | | Length = 462 |
| 889 | 2024889 | 3'Rgd(764-766) |
| 890 | 2024890 | Tyr_Phospho_Site(750-757) |
| 891 | 2024891 | 6E-39 >emb\|CAA63008\| (X91915) |
| | | LEA 0113 homologue type1 |
| | | [Arabidopsis thaliana] Length = 158 |
| 892 | 2024892 | 1E-72 >gi\|3413705 (AC004747) |
| | | glycine dehydrogenase [Arabidopsis |
| | | thaliana] Length = 1044 |
| 893 | 2024893 | Tyr_Phospho_Site(738-745) |
| 894 | 2024894 | Tyr_Phospho_Site(223-229) |
| 895 | 2024895 | 1E-53 >gb\|AAD27909.1\|AC007213_7 |
| | | (AC007213) receptor protein kinase |
| | | [Arabidopsis thaliana] Length = 851 |
| 896 | 2024896 | 8E-65 >gi\|3687245 (AC005169) |
| | | ribosomal protein [Arabidopsis thaliana] |
| | | Length = 129 |
| 897 | 2024897 | Pkc_Phospho_Site(19-21) |
| 898 | 2024898 | Pkc_PhosphoSite(2-4) |
| 899 | 2024899 | Tyr_Phospho_Site(299-305) |
| 900 | 2024900 | Tyr_Phospho_Site(629-636) |
| 901 | 2024901 | 1E-15 >gb\|AAD37511.1\|AF139098_1 |
| | | (AF139098) zinc finger protein [Arabidopsis |
| | | thaliana] Length = 186 |
| 902 | 2024902 | Tyr_Phospho_Site(194-201) |
| 903 | 2024903 | 4E-65 >emb\|CAB36774.1\| (AL035524) |
| | | senescence-associated protein-like |
| | | [Arabidopsis thaliana] Length 263 |
| 904 | 2024904 | Tyr_Phospho_Site(442-449) |
| 905 | 2024905 | Somatotropin_2(1615-1632) |
| 906 | 2024906 | 1E-99 >sp\|Q42569\|C901_ARATH CYTOCHROME |
| | | P450 90A1 >gi\|1076315\|pir\|\|S55379 |
| | | cytochrome P450 - Arabidopsis |
| | | thaliana >gi\|853719\|emb\|CAA60793\| |
| | | (X87367) CYP90 protein [Arabidopsis |
| | | thaliana] >gi\|871988\|emb\|CAA60794\| |
| | | (X87368) CYP9O protein [Arabidopsis thaliana] |
| | | Length = 472 |
| 907 | 2024907 | 1E-45 >gi\|2829899 (AC002311) |
| | | similar to ripening-induced protein, |
| | | gp\|AJ001449\|2465015 and major#latex protein, |
| | | gp\|X91961\|1107495 [Arabidopsis |
| | | thaliana] Length = 160 |
| 908 | 2024908 | 1E-119 >gi\|1895084 (U89897) |
| | | golgi associated protein se-wap41 [Zea |
| | | mays] Length = 364 |
| 909 | 2024909 | 9E-90 >sp\|P15459\|2SS3_ARATH 2S |
| | | SEED STORAGE PROTEIN 3 |
| | | PRECURSOR (25 ALBUMIN STORAGE |
| | | PROTEIN) >gi\|68855\|pir\|\|NWMU3 2S |
| | | albumin 3 precursor-Arabidopsis |
| | | thaliana >gi\|166616 (M22033) albumin 2S |
| | | subunit 3 precursor [Arabidopsis |
| | | thaliana] >gi\|395201 \|emb\|CAA80868\| |
| | | (Z24744) 2S albumin isoform 3 [Arabidopsis |
| | | thaliana] >gi\|4490712\|emb\|CAB38846.1\| |
| | | AL035680 NWMU3-2S albumin 3 recursor |
| | | [Arabidosis thaliana] Length = 164 |
| 910 | 2024910 | 1E-109 >gb\|AAD20398\| (AC007019) |
| | | ribonucleoside-diphosphate |
| | | reductase large subunit [Arabidopsis thaliana] |
| | | Length = 816 |
| 911 | 2024911 | Pkc_Phospho_Site(7-9) |
| 912 | 2024912 | 4E-31 >sp\|P41153\|HSF8_LYCPE HEAT |
| | | SHOCK FACTOR PROTEIN HSF8 |
| | | (HEAT SHOCK TRANSCRIPTION FACTOR 8) |
| | | (HSTF 8) (HEAT STRESS TRANSCRIPTION |
| | | FACTOR) >gi\|100264 \|pir\|\|S25481 |
| | | heat shock transcription factor 8 - |
| | | Peruvian tomato >gi\|19492\|em |
| 913 | 2024913 | 2E-82 >gi\|3176726 (AC002392) |
| | | serine proteinase [Arabidopsis thaliana] |
| | | Length = 815 |
| 914 | 2024914 | Tyr_Phospho_Site(235-243) |
| 915 | 2024915 | 2E-66 >sp\|P46602\|HAT3_ARATH HOMEO- |
| | | BOX-LEUCINE ZIPPER PROTEIN |
| | | thaliana] >gi\|549890 (U09339) |
| | | homeobox protein [Arabidopsis thaliana] |
| | | Length = 315 |
| 916 | 2024916 | 1E-119 ) >gb\|AAD40885.1\|AF091713_1 |
| | | (AF091713) cellulose synthase catalytic |
| | | subunit [Arabidopsis thaliana] Length = 1026 |
| 917 | 2024917 | 2E-30 >sp\|Q24595\|XPC_DROME DNA- |
| | | REPAIR PROTEIN COMPLEMENTING |
| | | XP-C CELLS HOMOLOG (XERODERMA |
| | | PIGMENTOSUM GROUP C |
| | | COMPLEMENTING PROTEIN HOMOLOG) |
| | | (XPCDM) >gi\|630881 \|pir\|\|S42402 |
| | | xeroderma pigmentosum group C complementing |
| | | factor - fruit fly (Drosophila |
| | | melanogaster) >gi\|434008\|emb\|CA |
| 918 | 2024918 | 9E-11 >sp\|P53582\|AMP1_HUMAN METH- |
| | | IONINEAMINOPEPTIDASE 1 |
| | | (METAP 1) (PEPTIDASE M 1) |
| | | (KIAA0094) >gi\|577315\|dbj\|BAA07679\| |
| | | (D42084) KIAA0094 gene product is related to |
| | | S.cerevisiae methionine aminopeptidase. |
| | | [Homo sapiens] |
| 919 | 2024919 | 3E-90 >gi\|2317912 (U89959) |
| | | cathepsin B-like cysteine proteinase |
| | | [Arabidopsis thaliana] Length = 357 |
| 920 | 2024920 | 5E-53 >emb\|CAA64636\| (X95343) |
| | | hypersensitivity-related gene [Nicotiana |
| | | tabacum] Length = 460 |
| 921 | 2024921 | 4E-79 >gb\|AAD50034.1\|AC007651_29 |
| | | (AC007651) Very similar to SRG1 |
| | | [Arabidopsis thaliana] Length = 346 |
| 922 | 2024922 | 1E-119 >pir\|\|S51480 drought-induced protein Dr4 - |
| | | Arabidopsis thaliana >gi\|469114\|emb\|CAA55323\| |
| | | (X78586) Dr4 [Arabidopsis thaliana], |
| | | trypsin inhibitor Length = 209 |
| 923 | 2024923 | Pkc_PhosphoSite(189-191) |
| 924 | 2024924 | 3E-71 >ref\|NP_006692.1\|PD1M1\| |
| | | similar to S. pombe dim1+ >gi\|2565275 |
| | | (AF023611) Dimip homolog [Homo sapiens] |
| | | Length = 142 |
| 925 | 2024925 | 1E-60 >gi\|1707011 (U78721) |
| | | auxin-repressed protein isolog [Arabidopsis |
| | | thaliana] Length = 108 |
| 926 | 2024926 | 2E-49 >gi\|2829923 (AC002291) |
| | | Similar to uridylyl transferases |
| | | [Arabidopsis thaliana] Length = 453 |
| 927 | 2024927 | Pkc_Phospho_Site(33-35) |
| 928 | 2024928 | 7E-74 >pir\|\|S57951 beta- |
| | | fructofuranosidase (EC 3.2.1.26) - Arabidopsis |
| | | thaliana (fragment) >gi\|899153\|emb\|CAA61624\| |
| | | (X89454) beta-fructofuranosidase |
| | | [Arabidopsis thaliana] Length = 562 |
| 929 | 2024929 | 8E-36 >gi\|3228664 (AF069986) |
| | | nitrilase and fragile histidine triad fusion |
| | | protein NitFhit [Caenorhabditis elegans] |
| | | Length = 440 |
| 930 | 2024930 | 4E-58 >gi\|2246621 (AF004393) |
| | | salt-stress induced tonoplast intrinsic |
| | | protein [Arabidopsis thaliana] Length = 273 |
| 931 | 2024931 | Tyr_Phospho_Site(315-322) |
| 932 | 2024932 | Tyr_Phospho_Site(784-791) |
| 933 | 2024933 | 6E-69 >emb\|CAA18734.1\| (AL022604) |
| | | cysteine proteinase-like protein |
| | | (Arabidopsis thaliana] Length = 355 |
| 934 | 2024934 | 1E-80 >sp\|PS3780\|METC_ARATH CYS- |
| | | TATHIONINE BETA-LYASE |
| | | PRECURSOR (CBL) (BETA- |
| | | CYSTATHIONASE) (CYSTEINE |
| | | LYASE) >gi\|2129567\|pir\|\|S61429 |
| | | cystathionine beta-lyase (EC 4.4.1.8) - Arabidopsis |
| | | thaliana >gi\|704397 (L40511) cystathionine |
| | | beta-lyase [Arabidopsis thaliana] |
| | | Length = 464 |
| 935 | 2024935 | Pkc_Phospho_Site(28-30) |
| 936 | 2024936 | Tyr_Phospho_Site(8-15) |
| 937 | 2024937 | 4E-88 >gi\|2642430 (AC002391) |
| | | AP2 domain containing protein |
| | | [Arabidopsis thaliana] Length = 176 |
| 938 | 2024938 | 1E-108 >emb\|CAB10321.1\| (Z97338) |
| | | UFD1 like protein [Arabidopsis |
| | | thaliana] Length = 778 |
| 939 | 2024939 | Tyr_Phospho_Site(545-553) |
| 940 | 2024940 | 1E-77 >emb\|CAA55358\| (X78703) |
| | | catechol O-methyltransferase [Vanilla |
| | | planifolia] Length = 363 |
| 941 | 2024941 | 1E-108 >gi\|3236251 (AC004684) |
| | | phosphoribosylaminoimidazole |
| | | carboxylase [Arabidopsis thaliana] Length = 645 |
| 942 | 2024942 | Tyr_Phospho_Site(779-787) |
| 943 | 2024943 | Pkc_Phospho_Site(62-64) |
| 944 | 2024944 | 2E-94 >sp\|P42770\|GSHC_ARATH GLU- |
| | | TATHIONE REDUCTASE, |
| | | CHLOROPLAST PRECURSOR (GR) |
| | | (GRASE) >gi\|451198\|dbj\|BAA03137\| |
| | | (D14049) glutathione reductase precursor [Arabidopsis |
| | | thaliana] >gi\|1944448\|dbj\|BAA19653\| |
| | | (D89620) glutathione reductase precursor |
| | | [Arabidopsis thaliana] >gi\|740576\|prf\|\|2005376A |
| | | glutathione reductase |
| | | [Arabidopsis thaliana] Length = 565 |
| 945 | 2024945 | 9E-56 >sp\|P02300\|H3_PEA HISTONE |
| | | H3 >gi\|81849\|pir\|\|S04520 histone H3 (clone pH3c-1) - |
| | | alfalfa >gi\|82609\|pir\|\|A26014 histone H3 - |
| | | wheat >gi\|19607\|emb\|CAA31964\| (X13673) |
| | | histone H3 (AA 1-136) [Medicago |
| | | sativa] >gi\|19609\|emb\|CAA319651\| |
| | | (X13674) histone H3 (AA 1-136) [Medicago |
| | | sativa] >gi\|21797\|emb\|CAA25451\| |
| | | (X00937) H3 histone [Triticum aestivum] >gi\|488565 |
| | | (U09459) histone H3.1 [Medicago |
| | | sativa] >gi\|2565419 (AF026803) histone H3 |
| | | [Onobrychis viclifolia] Length = 136 |
| 946 | 2024946 | 1E-23 >sp\|P72749\|TYPA_SYNY3 GTP- |
| | | BINDING PROTEIN TYPA/BIPA |
| | | HOMOLOG >gi\|1651837\|dbj\|BAA16764\| |
| | | (D90900) elongation factor EF-G |
| | | [Synechocystis sp.] Length = 597 |
| 947 | 2024947 | Tyr_Phospho_Site(1486-1494) |
| 948 | 2024948 | Tyr_Phospho_Site(31-38) |
| 949 | 2024949 | 2E-62 >emb\|CAB37514\| (AL035540) |
| | | farnesylated protein (ATFP6) |
| | | [Arabidopsis thaliana] Length = 153 |
| 950 | 2024950 | 7E-53 >emb\|CAA17529.1\| (AL021960) |
| | | UV-damaged DNA-binding protein- |
| | | like [Arabidopsis thalianall Length = 1102 |
| 951 | 2024951 | Pkc_Phospho_Site(65-67) |
| 952 | 2024952 | Tyr_Phospho_Site(655-662) |
| 953 | 2024953 | Tyr_Phospho_Site(498-505) |
| 954 | 2024954 | 8E-35 >gb\|AAD32652.1\|AF139188_1 |
| | | (AF139188) HCF106 [Arabidopsis thaliana] |
| | | Length = 260 |
| 955 | 2024955 | 6E-93 ) >pir\|\|S65046 1,4-alpha- |
| | | glucan branching enzyme (EC 2.4.1.18) |
| | | isoform SBE2.2 precursor - Arabidopsis thaliana |
| | | (fragment) >gi\|726490 (U22428) |
| | | starch branching enzyme class II (Arabidopsis thaliana] |
| | | Length = 800 |
| 956 | 2024956 | Tyr_Phospho_Site(549-556) |
| 957 | 2024957 | 9E-85 ) >gb\|AAC17823.1\| (AC004401) |
| | | casein kinase II catalytic subunit |
| | | [Arabidopsis thaliana] Length = 432 |
| 958 | 2024958 | 3E-46 >gi\|4185505 (AF101038) |
| | | nonspecific lipid-transfer protein |
| | | precursor [Brassica napus] Length = 112 |
| 959 | 2024959 | 2E-84 >sp\|Q96286\|DCAM_ARATH S- |
| | | ADENOSYLMETHIONINE |
| | | DECARBOXYLASE PROENZYME (ADOMETOC) |
| | | (SAMDC) >gi\|1531763\|emb\|CAA69073\| |
| | | (Y07765) S-adenosylmethionine decarboxylase |
| | | [Arabidopsis thaliana] Length = 366 |
| 960 | 2024960 | 1E-111 >emb\|CAB45311.1\| (AL079344) |
| | | arginine methyltransferase (pam1) |
| | | [Arabidopsis thaliana] Length = 390 |
| 961 | 2024961 | 2E-57 >gb\|AAD29776.1\|AF07402_8 |
| | | (AF074021) symbiosis-related protein |
| | | [Arabidopsis thaliana] Length = 122 |
| 962 | 2024962 | 1E-116 >gi\|3738287 (AC005309) |
| | | glutathione s-transferase, GST6 |
| | | [Arabidopsis thaliana] Length = 263 |
| 963 | 2024963 | 1E-118 ) >gi\|3540178 (AC004122) |
| | | calcium-transporting ATPase |
| | | [Arabidopsis thaliana] Length = 985 |
| 964 | 2024964 | Pkc_PhosphoSite(54-56) |
| 965 | 2024965 | 4E-81 ) >gi\|2088653 (AF002109) |
| | | Hs1pro-1 related protein isolog |
| | | [Arabidopsis thaliana] Length = 435 |
| 966 | 2024966 | 1E-12 >emb\|CAB57334.1\| (AL121741) |
| | | WD repeat protein. |
| | | [Schizosaccharomyces pombe] Length = 341 |
| 967 | 2024967 | Tyr_Phospho_Site(325-332) |
| 968 | 2024968 | 1E-135 ) >gb\|AAC50037.1\| (U97200) |
| | | cobalamin-independent methionine |
| | | synthase [Arabidopsis thaliana] Length = 765 |
| 969 | 2024969 | Tyr_Phospho_Site(107-113) |
| 970 | 2024970 | Tyr_Phospho_Site(1025-1032) |
| 971 | 2024971 | 5E-63 >gi\|3176687 (AC003671) |
| | | Strong similarity to trehalose-6- |
| | | phosphate synthase homolog from A. thaliana chromosome 4 |
| | | contig gb\|Z97344. ESTs gb\|H37594, gb\|R65023, |
| | | gb\|H37578 and gb\|R64855 come from this gene. |
| | | [Arabidopsis thaliana] Length = 826 |
| 972 | 2024972 | 1E-22 >gb\|AAD46412.1\|AF096262_1 |
| | | (AF096262) ER6 protein [Lycopersicon |
| | | esculentum] Length = 168 |
| 973 | 2024973 | Pkc_Phospho_Site(25-27) |
| 974 | 2024974 | 8E-74 >sp\|P46297\|RS23_FRAAN 40S |
| | | RIBOSOMAL PROTEIN S23 |
| | | (S12) >gi\|1362041\|pir\|\|S56673 ribosomal |
| | | protein S23.e, cytosolic (clone RJ3) - garden |
| | | strawberry >gi\|643074 (U19940) 40S ribosomal protein |
| | | s12 [Fragaria x ananassa] Length = 142 |
| 975 | 2024975 | Tyr_Phospho_Site(204-210) |
| 976 | 2024976 | 1E-102 >sp\|P30184\|AMPL_ARATH CYTOSOL |
| | | AMINOPEPTIDASE (LEUCINE |
| | | AMINOPEPTIDASE) (LAP) |
| | | (LEUCYL AMINOPEPTIDASE) (PROLINE |
| | | AMINOPEPTIDASE) (PROLYL AMINO- |
| | | PEPTIDASE) >gi\|99683\|pir\|\|S22399 |
| | | leucyl aminopeptidase (EC 3.4.11.1) - Arabidopsis |
| 977 | 2024977 | 1E-63 >gb\|AAD39321.1\|AC007258_10 |
| | | (AC007258) disease resistance protein |
| | | [Arabidopsis thaliana] Length 906 |
| 978 | 2024978 | Pkc_Phospho Site(1 73-175) |
| 979 | 2024979 | 2E-82 >sp\|P51823\|ARF_ORYSA ADP- |
| | | RIBOSYLATION |
| | | FACTOR >gi\|1132483\|dbj\|BAA04607\|(D17760) |
| | | ADP-ribosylation factor [Oryza sativa] |
| | | Length = 181 |
| 980 | 2024980 | 8E-65 >emb\|CAB4l7l2.1\| (AL049730) |
| | | pollen-specific protein [Arabidopsis |
| | | thaliana] Length = 587 |
| 981 | 2024981 | 1E-28 >emb\|CAB56770.1\| (AJ242957) |
| | | SPL1-Related2 protein [Arabidopsis |
| | | thaliana] Length = 812 |
| 982 | 2024982 | Tyr_Phospho_Site(99-105) |
| 983 | 2024983 | 1E-28 >emb\|CAA28192\| (X04507) |
| | | actin A3 [Bombyx mori] Length = 376 |
| 984 | 2024984 | 1E-74 >gb\|AAD28760.1\|AF130253_1 |
| | | (AF130253) membrane related protein CP5 |
| | | [Arabidopsis thaliana] Length = 387 |
| 985 | 2024985 | 3E-38 >gi\|3790587 (AF079182) |
| | | RING-H2 finger protein RHF2a |
| | | [Arabidopsis thaliana] Length = 375 |
| 986 | 2024986 | 1E-94 >emb\|CAB37523\| (AL035540) |
| | | thaumatin-like protein [Arabidopsis |
| | | thaliana] Length = 236 |
| 987 | 2024987 | Pkc_Phospho_Site(52-54) |
| 988 | 2024988 | 1E-112 ) >gb\|AAD25856.1\|AC007197_9 |
| | | (AC007197) dynamin-like protein ADL2 |
| | | [Arabidopsis thaliana] Length = 782 |
| 989 | 2024989 | 6E-20 >gi\|3193316 (AF069299) |
| | | contains similarity to nucleotide sugar |
| | | epimerases [Arabidopsis thaliana] |
| | | Length = 430 |
| 990 | 2024990 | 6E-52 >sp‥O23654\|VATA_ARATH VACUOLAR |
| | | ATP SYNTHASE CATALYTIC |
| | | SUBUNIT A (V-ATPASE 69 KD |
| | | SUBUNIT) >gi\|2266990 (U65638) vacuolar type |
| | | ATPase subunit A [Arabidopsis |
| | | thaliana] >gi\|3834305 (AC005679) Identical to |
| | | gb\|U65638 Arabidopsis thaliana vacuolar type ATPase |
| | | subunit A mRNA. ESTs gb\|N96435, gb\|N96106, |
| | | gb\|N96189, gb\|N96091, gb\|AA042286, gb\|F14324, |
| | | gb\|W43643, gb\|N96027, gb\|N96299, gb\|R29943, |
| | | gb\|T43460, gb\|T43544, gb\|T22472... Length = 623 |
| 991 | 2024991 | Tyr_Phospho_Site(960-967) |
| 992 | 2024992 | Pkc_Phospho_Site(30-32) |
| 993 | 2024993 | 3E-78 >sp\|P29510\|TBA2_ARATH TUBULIN |
| | | ALPHA-2/ALPHA-4 |
| | | CHAIN >gi\|320183\|pir\|\|JQ1594 tubulin |
| | | alpha chain - Arabidopsis thaliana >gi\|166914 |
| | | (M84696) apha-2 tubulin [Arabidopsis |
| | | thaliana] >gil166916 (M84697) alpha-4 |
| | | tubulin [Arabidopsis thaliana] Length = 450 |
| 994 | 2024994 | 3'Pkc_Phospho_Site(40-42) |
| 995 | 2024995 | Pkc_Phospho_Site(21-23) |
| 996 | 2024996 | 2E-61 >pir\|\|S58275 keto- |
| | | conazole resistent protein - Arabidopsis |
| | | thaliana >gi\|928938\|emb\|CAA61433\| |
| | | (X89036) ketoconazole resistent protein Arabidopsis |
| | | thaliana] Length = 140 |
| 997 | 2024997 | Tyr_Phospho_Site(66-72) |
| 998 | 2024998 | 7E-42 >gi\|1899188 (U90212) |
| | | DNA binding protein ACBF [Nicotiana |
| | | tabacum] Length 428 |
| 999 | 2024999 | 5E-19 >gb\|AAD47084.1\|AF165883_1 |
| | | (AF165883) prefoldin subunit 2 [Homo |
| | | sapiens] Length = 155 |

What is claimed is:

1. A nucleic acid comprising a sequence capable of hybridizing under stringent conditions to a sequence set forth in SEQ ID NO:1 to 999, or a fragment thereof.

2. A vector comprising the nucleic acid of claim 1.

3. The vector of claim 2, wherein said vector comprises regulatory elements for expression, operably linked to said sequence.

4. A polypeptide encoded by the nucleic acid of claim 1.

5. A nucleic acid comprising: an ATG start codon; an optional intervening sequence; a coding sequence capable of hybridizing under stringent conditions as set forth in SEQ ID NO:1 to 999; and an optional terminal sequence, wherein at least one of said optional sequences is present, and wherein:
ATG is a start codon;
said intervening sequence comprises one or more codons in-frame with said coding sequence, and is free of in-frame stop codons; and
said terminal sequence comprises one or more codons in-frame with said coding sequence, and a terminal stop codon.

6. The nucleic acid of claim 5, wherein said nucleic acid is expressed in Arabidopsis thaliana.

7. The nucleic acid of claim 5, wherein said nucleic acid encodes a plant protein.

8. The nucleic acid of claim 7, wherein said plant is a dicot.

9. The nucleic acid of claim 8, wherein said dicot is Arabidopsis thaliana.

10. The nucleic acid of claim 7, wherein said plant protein is a naturally occurring plant protein.

11. The nucleic acid of claim 7, wherein said plant protein is a genetically modified plant protein.

12. The nucleic acid of claim 5, wherein said nucleic acid encodes a fusion protein comprising an Arabidopsis thaliana protein and a fusion partner.

13. The nucleic acid of claim 5, wherein said nucleic acid encodes a fusion protein comprising of plant protein and a fusion partner.

14. A transgenic plant comprising an exogenous nucleic acid, wherein said nucleic acid comprises transcription regulatory sequences operably linked to a sequence capable of hybridizing under stringent conditions to a sequence set forth in SEQ ID NO:1 to 999 or a fragment thereof, wherein said sequence is expressed in cells of said plant.

15. The transgenic plant of claim 14, wherein said plant is regenerated from transformed embryogenic tissue.

16. The transgenic plant of claim 14, wherein said plant is a progeny of one or more subsequent generations from transformed embryogenic tissue.

17. The transgenic plant of claim 14, wherein said sequence capable of hybridizing under stringent conditions to a sequence set forth in SEQ ID NO:1 to 999 encodes a plant protein.

18. The transgenic plant of claim 14, wherein said plant protein is a naturally occurring plant protein.

19. The transgenic plant of claim 14, wherein said plant protein is a genetically altered plant protein.

20. The transgenic plant of claim 14, wherein said sequence expressed in cells of said plant is an anti-sense sequence.

21. The transgenic plant of claim 14, wherein said sequence expressed in cells of said plant is a sense sequence.

22. The transgenic plant of claim 14, wherein said sequence is selectively expressed in specific tissues of said plant.

23. The transgenic plant of claim 14, wherein said specific tissue is selected from the group consisting of leaves, stems, roots, flowers, tissues, epicotyls, meristems, hypocotyls, cotyledons, pollen, ovaries, cells, and protoplasts.

24. A genetically modified cell, comprising an exogenous nucleic acid, wherein said nucleic acid comprises transcription regulatory sequences operably linked to a sequence capable of hybridizing under stringent conditions to a sequence set forth in SEQ ID NO:1 to 999, wherein said sequence is expressed in cells of said plant.

25. A method of screening a candidate agent for its biological effect; the method comprising:
combining said candidate agent with one of:
a genetically modified cell according to claim 24, a transgenic plant according to claim 14, or a polypeptide according to claim 4; and
determining the effect of said candidate agent on said plant, cell or polypeptide.

26. A nucleic acid array comprising at least one nucleic acid as set forth in SEQ ID NO:1-999 stably bound to a solid support.

27. An array comprising at least one polypeptide encoded by a nucleic acid as set forth in SEQ ID NO:1-999, stably bound to a solid support.
